(19) United States
(12) Patent Application Publication
(10) Pub. No.: US 2024/0011108 A1
(43) Pub. Date: Jan. 11, 2024

(54) MASS SPECTROMETRY-BASED METHODS AND KITS FOR NUCLEIC ACID DETECTION AND DISEASE DIAGNOSTIC

(71) Applicant: YYZ PHARMATECH INC., Toronto (CA)

(72) Inventors: John G. Marshall, Toronto (CA); Ming Miao, Toronto (CA)

(21) Appl. No.: 18/033,857

(22) PCT Filed: Oct. 26, 2021
(86) PCT No.: PCT/CA2021/051510

§ 371 Date: Apr. 26, 2023

Related U.S. Application Data
(60) Provisional application No. 63/105,554, filed on Oct. 26, 2020.

(51) Int. Cl. C12Q 1/70; C12Q 1/6851; C12Q 1/6825

(52) U.S. Cl. CPC C12Q 1/701; C12Q 1/6851; C12Q 1/6825

(57) ABSTRACT

The present disclosure relates to methods of detecting nucleic acid using mass spectrometry. The present disclosure further relates to methods of detecting nucleic acids of and/or diagnosing diseases such as HIV and COVID-19 by detecting presence of target nucleic acid molecule in samples. The present disclosure also relates to kits for detecting nucleic acid and for detecting nucleic acids of and/or diagnosing diseases such as HIV and COVID-19.

### RELATED APPLICATIONS

This PCT application claims priority to U.S. Application Ser. No. 63/105,554, filed Oct. 26, 2020, herein incorporated by reference.

### FIELD

The present disclosure pertains to methods of and kits for detecting and measuring a target nucleic acid using a mass spectrometric method. Further, the present disclosure relates to methods and kits for disease diagnostics.

### INTRODUCTION

The capacity to accurately detect and quantify biomolecules is of great importance in multiple fields including basic biochemistry research, diagnostic and therapeutic medicine as well as water and food safety. Many potential diagnostic DNA molecules and therapeutic proteins at the edge of detection by present methods need to be absolutely quantified. The discovery of biologically important nucleic acids by semi quantitative “counting” methods such as polymerase chain reaction (PCR) amplification and DNA sequencing on polystyrene oligo synthesis microbeads has revealed important molecules (Consortium, 2011) that need to be absolutely quantified alongside standards by linear and Gaussian hybridization assays. Current techniques such as PCR are not able to accurately quantify molecules at these levels of zeptomole (10⁻²¹) to yoctomole (10⁻²⁴) amounts under assay (Rutledge, 2003).

PCR (Chin, 2013) has been used to detect as little as a single polymerase template but is non-linear, may show false negative results, has large quantitative errors, and the mathematical procedure to extract absolute quantification from PCR reactions is daunting (Rutledge, 2003). Analysis of HIV and other animal viruses by PCR has a significant false negative rate (Xie, 2020; Xiao, 2020). A wide range of sensitivity values have been reported for Hybridization and Hybridization Chain reaction (Basiri, 2020; Santhanam, 2020, Doddapaneni, 2020; Jiao, 2020; Vermisoglou, 2020). A recent application of quantitative DNA based assays on solid supports may have reached the pico molar (pM) concentration range or using fluorescence that uses a broad absorption range, using electrochemical detection or TIRF that is not inherently linear and Gaussian or using schemes with multiple rounds of amplification by PCR or HCR followed by enzyme amplification that may show multiplication of error (Xu, 2016) Shi, Guo, Xiong and or ultrasensitive refences. In contrast mass spectrometry is more specific to a single mass to charge ratio instead of a broad spectrum, is inherently linear and Gaussian and can be amplified with one round of enzyme amplification to reach pM or lower concentration ranges.

Total internal reflectance of fluorescence (TIRF) can be used in the qualitative detection of nucleotides in DNA sequences (Vandamme, 1995). However, the signal is non-linear such that that calibration can be out by 1000 fold (Tobos, 2019; Tangemann, 1995) and relies on the aggregation of qualitative data that prevents computing of a safe detection limit (Rissin, 2010). Quantification from TIRF has practical limitations and was recently shown to provide results similarto those of enzyme amplification using horseradish peroxidase (HRP) (Li, 2017).

Mass spectrometry is a linear and Gaussian analytical technique (Razumienko, 2008; Bowden, 2012) that detects adenosine at 100 picomolar concentration (100 pM) where 1 microlitre injected (1 μL) corresponds to 100 attomole (100 amol) on column even prior to enzyme amplification (Florentinus, 2011; Onisko, 2007).

Liquid chromatography electrospray ionization tandem mass spectrometry (LC-ESI-MS/MS) has some powerful advantages compared to other methods that can directly detect proteins from blood to ng/ml levels without immunological or enzymatic amplification (Munge, 2005).

Immuno-Matrix Assisted Laser Desorption/Ionization (MALDI) directly analyzes immune complexes of proteins or peptides (Li, 2017) but has not been as useful for DNA. Moreover, its signal does not benefit from enzyme amplification and only reaches ng/ml sensitivity.

Similarly, liquid chromatography inductively coupled plasma mass spectrometry (LC-ICP-MS) may commonly reach ng/ml levels similar to the existing detection limits of ELISA (Shukla, 2013).

Existing electrochemical methods have been reported to reach the yoctomole range. However, the signal is not inherently linear or Gaussian (Saiki, 1985; Rissin, 2010).

UV/VIS detection is not as sensitive or specific as mass spectrometry; but the combination of enzyme amplification and UV/VIS detection powerfully increased the sensitivity of UV/VIS analysis. The use of enzyme amplification by alkaline phosphatase (AP), DNA polymerase, horse radish peroxidases or luciferase has increased the useful sensitivity of methods such as UV-VIS, ECL or fluorescent detection (Ronaghi, 1996; Chen, 1994; Florentinus-Mefailoski, 2014; Walt, 2013; Munge, 2005; Saiki, 1985; Sun, 2006; Shukla, 2013; Chin, 2013; Tobos, 2019; Vandamme, 1995; Tangemann, 1995; Tucholska, 2009; Li, 2017; Razumienko, 2008; Bowden, 2012; Florentinus-Mefailoski, 2015; Florentinus, 2011; Onisko, 2007).

Using enzyme linked immuno mass spectrometric assay (ELiMSA), proteins and antibodies have been previously absolutely quantified on polystyrene supports using 96-well plate with deoxycholate or N-octyl glucoside modified, LC-ESI-MS compatible protein interaction buffers (Florentinus-Mefailoski, 2014; Florentinus-Mefailoski, 2016; Florentinus-Mefailoski, 2014; Florentinus-Mefailoski, 2015). ELiMSA assay has been described in U.S. Pat. No. 9,964,538. Compared to direct measurement by traditional colorimetric enzyme linked immunosorbent assay (ELISA), which reaches nanogram amounts of proteins, ELiMSA has reached picogram sensitivity for the detection of protein using alkaline phosphatase streptavidin (APSA) enzyme conjugate that is detectable to 50 femtogram (Florentinus-Mefailoski, 2015).

Detection of prostate specific antigen (PSA) and antibodies using the APSA enzyme conjugate reached high yoctomole range on normal phase silica stationary phase (Florentinus-Mefailoski, 2014; Florentinus-Mefailoski, 2015; Florentinus-Mefailoski, 2016). Protein detection by ELiMSA was blind tested to show results that agreed with the commercial fluorescent and ECL systems at high concentrations, but was far more sensitive and continued to show linear quantification of far below 1 ng/ml (femto mole range) (Florentinus-Mefailoski, 2015).

The quantification of nucleic acid by mass spectrometry can be difficult. For example, buffers typically used with nucleic acid binding, hybridization and reaction contain salts such as NaCl to promote nucleic acid interaction. However, inorganic salts such as NaCl cannot easily be used in mass spectrometric measurements.

Accordingly, there is a need for linear and Gaussian assays for detection and quantification of nucleic acids that is sensitive at low concentrations, for example where the nucleic acid is present in a femto molar to atto molar concentration range, and/or preferably compatible with MS.

### SUMMARY

It has been shown presently that low concentrations of target nucleic acid molecule from for example biological samples or PCR reaction products can be sensitively and specifically detected and quantified. Methods described herein include methods that involve amplification using selective capture and/or detection oligonucleotide probes coupled with measuring an enzymatic activity of a reporter enzyme such as alkaline phosphatase (AP) for detection by mass spectrometric (MS) methods. Further, it has been shown that when at least one primer of a PCR reaction is functionalized with a secondary target moiety such as biotin, the PCR product can be directly detected and quantified with a reporter enzyme detection probe that binds to the secondary target moiety and that has enzymatic activity that amplifies the presence of the PCR product for detection by MS.

Further, it has been shown that volatile buffers can be used to replace salt such as NaCl in one or more buffers to minimize residual salt in MS analysis.

The methods of the present disclosure are useful as selective and sensitive diagnostic methods.

Accordingly, in one aspect, the present disclosure includes a method of detecting a target nucleic acid molecule comprising
a.
   i. incubating a sample putatively comprising the target nucleic acid molecule with a capture oligonucleotide probe that comprises a sequence complementary to the target nucleic acid molecule and that is attached to a solid phase, in a first binding solution, optionally wherein the solid phase is attached to the capture oligonucleotide probe through a linker; or
   ii. incubating a sample putatively comprising the target nucleic acid molecule with a solid phase to attach said sample/target nucleic acid molecule to said solid phase, in a first binding solution, optionally wherein the solid phase is attached to the sample/target nucleic acid molecule through a linker;
b. binding any target nucleic acid molecule to a detection oligonucleotide probe in a second binding solution under conditions for forming a target:detection complex;
c. incubating any target:detection complex with a reporter enzyme detection probe in a third volatile binding solution under conditions for forming a target:detection:enzyme complex, the third volatile binding solution substantially free of inorganic salt such as NaCl;
d. washing the solid phase to remove any unbound reporter enzyme detection probe with a washing solution;
e. incubating any target:detection:enzyme complex with a reporter enzyme detection probe substrate in a substrate reaction solution to generate one or more ionizable products; and
f. detecting at least one of the one or more ionizable products using mass spectrometry (MS),
wherein
   i. at least the third binding solution among the first binding solution, the second binding solution, and the third binding solution is substantially free of inorganic salt;
   ii. the washing solution is substantially free of inorganic salt;
   iii. the method further comprises cross-linking components of any target:detection:enzyme complex and the capture oligonucleotide probe prior to the optional step d) and the step e); and/or
   iv. the method further comprises separating the one or more ionizable products priorto detection using MS; and
wherein detection of the at least one of the one or more ionizable products is indicative of the sample comprising the target nucleic acid molecule.

The detection oligonucleotide probe can be a detection oligonucleotide primer. In such cases, the step comprises amplifying the target nucleic acid molecule with a detection oligonucleotide primer, in an amplification solution and binding any amplified target to the detection oligonucleotide probe in the second binding solution under conditions for forming a target:detection complex.

In another aspect, the present disclosure includes a method of quantifying the amount of a target nucleic acid molecule in a sample comprising the steps:
a. detecting a target nucleic acid molecule according to a method of the present disclosure; and
b. quantifying the amount of target nucleic acid molecule in the sample based on the intensity of the signal for one or more of the ionizable products detected by mass spectrometry.

In another aspect, the present disclosure includes a method of detecting a target nucleic acid molecule comprising
performing a nucleic acid amplification such as a polymerase chain reaction (PCR) or a hybridization chain reaction (HCR) or rolling circle reaction or other nucleic acid reaction on a test sample putatively comprising the target nucleic acid molecule with a modified primer and a second primer to obtain an amplified nucleic acid product, optionally a PCR product, comprising the modified primer, the modified primer being functionalized with a secondary target moiety or a reporter enzyme;
separating the amplified nucleic acid product from any unreacted modified primer;
when the modified primer is functionalized with the secondary target moiety, incubating the amplified nucleic acid product with a reporter enzyme detection probe in a first binding solution under conditions to form an amplified nucleic acid product:reporter enzyme complex, and removing any unbound reporter enzyme detection probe with a washing solution, the reporter enzyme detection probe comprising a secondary target binding moiety and a reporter enzyme;
incubating the amplified nucleic acid product or the amplified nucleic acid product:reporter enzyme complex with a reporter enzyme substrate in a substrate reaction solution to generate one or more ionizable products; and
detecting the one or more ionizable products using mass spectrometry (MS),
wherein when the modified primer is a forward primer, the second primer is a reverse primer, and
wherein when the modified primer is a reverse primer, the second primer is a forward primer.

In another aspect, the present disclosure includes a method of quantifying the amount of a target nucleic acid molecule in a test sample comprising the steps:
a. detecting the target nucleic acid molecule according to a method of detecting a target nucleic acid molecule of the present disclosure; and
b. quantifying the amount of target nucleic acid molecule in the test sample based on the intensity of the signal for one or more of the ionizable products detected by mass spectrometry.

In another aspect, the present disclosure includes a method of detecting HIV comprising a method of detecting a target nucleic acid molecule of the present disclosure, wherein the target nucleic acid molecule is a HIV nucleic acid molecule.

In another aspect, the present disclosure includes a method of detecting SARS-CoV2 comprising a method of detecting a target nucleic acid molecule of the present disclosure, wherein the target nucleic acid molecule is a SARS-CoV2 nucleic acid molecule.

In another aspect, the present disclosure includes a kit comprising:
i. a capture oligonucleotide probe, the capture oligonucleotide probe optionally bound of a solid phase, optionally through a linker;
ii. a binding solution comprising a volatile buffer and being substantially free of NaCl or comprising a cross-linking agent;
iii. a detection oligonucleotide probe, the detection oligonucleotide probe comprising an oligonucleotide and a secondary target moiety;
iv. a reporter enzyme detection probe, the reporter enzyme detection probe comprising a reporter enzyme and a secondary target binding moiety capable of binding the secondary target moiety; and/or
v. one or more of: a substrate, a solid phase, a standard, optionally a product ion standard, optionally for preparing a standard curve or tuning calibrant, a second binding solution, a third binding solution, a substrate reaction solution, ionization solution, quenching solution, optionally a second binding solution, detection probe solution, substrate reaction solution, quenching solution, ionization solution as defined herein.

In another aspect, the present aspect includes a kit comprising:
i. a modified primer, the modified primer being functionalized with a secondary target moiety or a reporter enzyme;
ii. a second primer;
iii. when the modified primer is functionalized with the secondary target moiety, a reporter enzyme detection probe, the reporter enzyme detection probe comprising a reporter enzyme and a secondary target binding moiety capable of binding the secondary target moiety; and
iv. one or more of: a substrate, a solid phase, a standard, optionally a product ion standard, optionally for preparing a standard curve or tuning calibrant, a binding solution, a second binding solution, a washing solution, a substrate reaction solution, ionization solution, quenching solution, optionally a binding solution, second binding solution, detection probe solution, substrate reaction solution, quenching solution, ionization solution as defined herein,
   wherein when the modified primer is a forward primer, the second primer is a reverse primer, and when the modified primer is a reverse primer, the second primer is a forward primer.

In another aspect, the present disclosure includes a nucleic acid of sequence selected from SEQ ID 2 to 37.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### DRAWINGS

An embodiment of the present disclosure will now be described in relation to the drawings in which:

FIG. 1 is a series of graphs that shows detection of a viral DNA performed using a capture oligonucleotide probe absorbed to 0.45 micron PVDF 96 well filter plates without vacuum. Panel A shows MS signal intensity at 268 [M+H]⁺ with blank (Tris buffer), no target nucleic acid molecule (0 Target) and 100 fmol target nucleic acid molecule (100 fmol Target). Panels B and C show scans from m/z 200 to 400. Panel D shows signal intensity of no target nucleic acid molecule compared to 100 fmol target nucleic acid molecule.

FIG. 2 is a series of graphs that shows detection of viral DNA performed in a polylysine coated 96 well polystyrene plate by NHS-PEG-NHS crosslinking capture oligonucleotide probe to the plate. Panel A shows MS signal intensity at 268 [M+H]⁺ with blank (Tris buffer), no target nucleic acid molecule (0 Target) and 100 fmol target nucleic acid molecule (100 fmol Target). Panel B and C show scans from m/z 200 to 400. Panel D shows signal intensity of no target nucleic acid molecule compared to 100 fmol target nucleic acid molecule.

FIG. 3 is a series of graphs that shows detection of viral DNA performed using capture oligonucleotide probe immobilized on the amine-reactive Nunc Immobilizer™ Amino 96 well polystyrene plate. Panel A shows MS signal intensity at 268 [M+H]⁺ with blank (Tris buffer), no target nucleic acid molecule (0 Target) and 100 fmol target nucleic acid molecule (100 fmol Target). Panel B and C show scans from m/z 200 to 400. Panel D shows signal intensity of no target nucleic acid molecule compared to 100 fmol target nucleic acid molecule.

FIG. 4 is a series of graphs that shows detection of viral DNA performed using capture oligonucleotide probe immobilized on NOS surface chemistry 96 well polystyrene reactive plates. Panel A shows MS signal intensity at 268 [M+H]⁺ with blank (Tris buffer), no target nucleic acid molecule (0 Target) and 100 fmol target nucleic acid molecule (100 fmol Target). Panel B and C show scans from m/z 200 to 400. Panel D shows signal intensity of no target nucleic acid molecule compared to 100 fmol target nucleic acid molecule.

FIG. 5 is a series of graphs that shows detection of viral DNA performed by capture oligonucleotide probe with 3′ links to polystyrene oligosynthesis beads in a 96 well PVDF filter plate. Panel A shows MS signal intensity at 268 [M+H]⁺ with blank (Tris buffer), no target nucleic acid molecule (0 Target) and 100 fmol target nucleic acid molecule (100 fmol Target). Panel B and C show scans from m/z 200 to 400. Panel D shows signal intensity of no target nucleic acid molecule compared to 100 fmol target nucleic acid molecule.

FIG. 6 is a series of graphs that shows detection of viral DNA performed on an amino-silylated cover glass by NHS-PEG-NHS crosslinking capture oligonucleotide probe to the glass. Panel A shows MS signal intensity at 268 [M+H]⁺ with blank (Tris buffer), no target nucleic acid molecule (0 Target) and 100 fmol target nucleic acid molecule (100 fmol Target). Panel B and C show scans from m/z 200 to 400. Panel D shows signal intensity of no target nucleic acid molecule compared to 100 fmol target nucleic acid molecule.

FIG. 7 is a series of graphs that shows results from optimization of NaCl in binding buffer for detecting HIV DNA with capture oligonucleotide probe bound to polystyrene oligosynthesis beads in 96-well plates. Panel A shows the average signal intensity of two injections at 268.2 m/z for different concentrations of NaCl. Panel B shows the signal intensity of each run. (B is blank with Tris buffer; 0, 0.05, 0.1, 0.5, 0.6, 0.7, 0.8, 0.9, 1.0, 1.5, and 2.0 indicates molar concentration of NaCl; 0 tgt is without any target nucleic acid molecule)

FIG. 8 is a series of graphs that shows results from optimization of ammonium bicarbonate in binding buffer for detecting HIV DNA during and after hybridization with capture oligonucleotide probe linked to polystyrene oligosynthesis beads in 96 well 0.45 um high binding PVDF filter plates. Panel A shows the average signal intensity of two injections at 268.2 m/z for different concentrations of ammonium bicarbonate and 1 M NaCl as comparison. Panel B shows the signal intensity of each run. (B is blank with Tris buffer; 0, 0.1, 0.5, 0.6, 0.7, 0.8, 0.9, 1.0, 1.5, 2.0, 2.5 indicates molar concentration of ammonium bicarbonate)

FIG. 9 is a series of graphs that shows results with different volatile buffers: ethanolamine, ammonium acetate, ammonium bicarbonate and triethyl ammonium bicarbonate substituted for 1.5M NaCl after hybridization for HIV DNA with capture oligonucleotide probe linked to polystyrene oligosynthesis beads in 96 well 0.45 um high binding PVDF filter plates. Panel A shows the average signal intensity of two injections at 268.2 m/z for different concentrations of various volatile buffers at the concentrations indicated on the X-axis and 1.5 M NaCl as comparison. Panel B shows the signal intensity of each run. (Rxn B is blank with Tris buffer)

FIG. 10 shows a graph showing MS signal intensities of HIV target DNA detection where different percentages of ethanol from 10 to 55% was used in the detection enzyme (APSA) binding step and the washing step (Columns 5 to 10). Columns 1 to 3 present results for negative controls including a reaction buffer control (column 1), a control where no salt in either detection enzyme (APSA) binding or washing step (column 2), and a control where 1.5 M NaCl was used in the hybridization step and no NaCl was used in the detection enzyme (APSA) binding step and the washing step (column 3). Column 4 presents a positive control, where 1.5 M NaCl was used in the hybridization step and in the detection enzyme (APSA) binding step and the washing step.

FIG. 11 shows a graph of MS signal intensity measured at m/z=267.74-268.74 in a SARS-CoV2 DNA detection assay, where 1.5 M NaCl, 2 M ethanolamine, 0.5 M trlethylammonlum bicarbonate, 2 M sucrose or 2 M glycine were used in the hybridization step, the detection enzyme (APSA) binding step and the washing step, or where 1.5 M NaCl was used in the hybridization step and 2 M ethanolamine, 0.5 M triethylammonium bicarbonate, 2 M sucrose or 2 M glycine were used in the detection enzyme (APSA) binding step and the washing step without NaCl.

FIG. 12 shows a polyacrylamide gel showing the PCR products of Example 11, where lane 1 corresponds to a direct load wide range molecular weight marker (5 μl), lane 2 corresponds to the PCR product obtained with primer combination 1, lane 4 corresponds to the PCR product obtained with primer combination 2, lane 6 corresponds to the PCR product obtained with primer combination 3, lane 4 corresponds to the PCR product obtained with primer combination 4, lanes 3, 5, 7, and 9 correspond to control runs where no template plasmid DNA was used, and lane 10 corresponds to negative control (4 μl of DNA loading buffer).

FIG. 13 shows a polyacrylamide gel showing the PCR amplification products of SARS-CoV2 using SARS-CoV2 Set 1 PCR Primers (SEQ ID Nos 2 and 3) and different amounts of template from 0 template (lane 0), trace template (lane 1) and a linear dilution series (0.1 ng, 1 ng, 10 ng, 50 ng, lanes 2 to 5). Lanes 6 to 10 show PCR product using 10 ng template and different amount of Mg²⁺ (2 mM, 2.5 mM, 3.0 mM, 3.5 mM, or 4.0 mM Mg²⁺ respectively).

FIG. 14 shows a graph of relative abundance of MS signal observed at m/z=267.74-268.74 detecting PCR products of SARS-CoV2 nucleocapsid gene by DNA detection assay of the present disclosure using the capture oligonucleotide probe (SEQ ID No. 6) attached to solid support at the 3′, and the 5′-biotinylated detection oligonucleotide (SEQ ID No. 5) with no template DNA (0 NC), PuC19 as template DNA (negative control for PCR), trace amount of plasmid carrying SARS-CoV2 nucleocapsid gene (T), and different amounts of plasmid carrying SARS-CoV2 nucleocapsid gene (10 fg, 100 fg, 1 pg, 10 pg, and 100 pg, corresponding to 10, 100, 1000, 10,000, and 100,000 respectively).

FIG. 15A shows an exemplified schematic illustrating detection of, for example, a hypothetical PCR or other products. Nucleic acid target can be DNA or RNA.

FIG. 15B shows a schematic illustrating detection of, for example, a PCR product. The primer may be represented by A/B indicating it may be untagged or tagged for example with biotin or presented by C/D indicating it may be unattached or attached to a solid surface.

FIG. 16 shows a graph of MS signal intensity measured at m/z=267.74-268.74 of a DNA detection assay where (i) the hybridization step and the washing step was performed in presence of salt, (ii) the hybridization step and the binding of APSA was performed in presence of salt, followed by cross-linking the target:detection:enzyme complex with glutaraldehyde (GA) prior to enzyme reaction in Tris buffer, (iii) the hybridization step and the binding of APSA were performed in presence of salt, followed by washing with a volatile washing solution comprising either ammonium bicarbonate buffer (AMBIC) or ethanolamine buffer (EA) and the enzyme reaction occurring in a volatile substrate reaction buffer comprising either ammonium bicarbonate buffer (AMBIC) or ethanolamine buffer (EA), or (iv) the hybridization step and the binding of APSA were performed in presence of salt, followed by the enzyme reaction occurring in presence of a polymer (PEG) or dextran sulfate sodium (DSS). 10 mM Tris was used as a negative control for the MS measurement. Zero target nucleic acid (0) is used as negative control for the DNA detection assay.

FIG. 17 shows a flowchart illustrating exemplary methods of the present disclosure.

FIG. 18 shows a graph of MS signal intensity (log scale, y-axis, intensity m/z=268) of HIV DNA detection assay at different concentrations of target nucleotide acid molecule (log scale, x-axis) of log 10 (attomolar+1) i.e. from 0 to 100 picomolar concentration where 1 microlitre was injected.

FIG. 19 shows a graph of MS signal intensity (log scale, y-axis, m/z=268) of SARC-CoV2 DNA detection assay at different concentrations of target nucleotide acid molecule (log scale, x-axis) of log 10 (picomolar+1) from 0 to 100 nM concentration where 1 micro litre was injected on to the HPLC column (i.e. 0 to 100 femtomole on column).

FIG. 20 shows a graph of MS signal intensity at m/z 136 at different concentrations of HIV DNA target nucleic acid molecule (1 pM to 500 pM).

FIG. 21 shows a graph of MS signal intensity at m/z 136 at different concentrations of SARS-CoV 2 target nucleic acid molecule (100 fM to 10 nM).

FIG. 22 shows a graph of MS signal intensity at m/z 136 at different concentrations of STEC target nucleic acid molecule (1 pM to 1 nM).

FIG. 23 shows a graph of MS signal intensity at m/z 136 at different concentrations of hemolysin target nucleic acid molecule (1 pM to 1 nM).

FIG. 24A shows a graph of MS signal intensity at m/z 136 at different concentrations of HIV 258 nt PCR product target nucleic acid molecule.

FIG. 24B shows an image of a GelRed stained agarose gel showing PCR reactions using increasing amounts of HIV plasmid.

FIG. 24 C shows a graph quantitation of bands in FIG. 24B.

FIG. 25 shows a graph of MS signal intensity at m/z 136 at different concentrations of SARS-CoV-2 target nucleic acid molecule.

FIG. 26 shows a graph of MS signal intensity at m/z 136 at different concentrations of HIV target nucleic acid molecule (100 fM to 100 nM).

FIG. 27 shows a graph of MS signal intensity at m/z 268 at different concentrations of SARS-CoV-2 target nucleic acid molecule (1 pM to 1 μM) where the capture is bound to PVDF.

FIG. 28A shows an image of gels where the upper panel showing PCR products produced using biotinylated HIV forward primer 3 and unlabelled HIV reverse primer 3 and where the lower panel showing PCR products produced using unlabelled HIV forward primer 3 and biotinylated HIV reverse primer 3.

FIG. 28B shows a graph of MS signal intensity at m/z 136 at different concentrations of HIV template using biotin labelled HIV forward primer and unlabelled HIV reverse primer.

FIG. 28C shows a graph of MS signal intensity at m/z 136 at different concentrations of HIV template using unlabelled HIV forward primer and biotin labelled HIV reverse primer.

FIG. 29 shows a graph of MS signal intensity at m/z 136 for HIV synthetic target immobilized on PDVF.

FIG. 30A shows an image of a gel showing various COVID-19 PCR reactions.

FIG. 30B shows an image of a gel showing COVID-19 PCR reactions at different concentrations of COVID-19 template.

### DESCRIPTION OF VARIOUS EMBODIMENTS

### I. Definitions

Unless otherwise indicated, the definitions and embodiments described in this and other sections are intended to be applicable to all embodiments and aspects of the present disclosure herein described for which they are suitable as would be understood by a person skilled in the art.

The term “or” “and/or” as used herein means that the listed items are present, or used, individually or in combination. In effect, this term means that “at least one of” or “one or more” of the listed items is used or present.

As used in the present disclosure, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

As used in this disclosure and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art.

The term “amine” or “amino,” as used herein, whether it is used alone or as part of another group, refers to groups of the general formula NR′R″, wherein R′ and R″ are each independently selected from hydrogen or C₁₋₆alkyl.

The term “atm” as used herein refers to atmosphere.

The term “MS” as used herein refers to mass spectrometry.

The term “aq.” as used herein refers to aqueous.

MeOH as used herein refers to methanol.

MeCN as used herein refers to acetonitrile.

HCl as used herein refers to hydrochloric acid.

μwave as used herein refers to a microwave reaction vessel.

LCMS as used herein refers to liquid chromatography-mass spectrometry.

TRIS as used herein refers to tris(hydroxymethyl)aminomethane.

EDTA as used herein refers to ethylenediaminetetraacetic acid.

The term “adenosine monophosphate” or “AMP” as used herein means a compound having the structure:

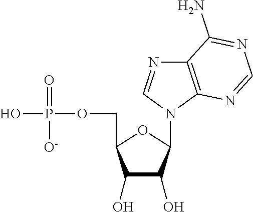 [SMILES: Nc1ncnc2c1ncn2[C@@H]1O[C@H](CO[P@SP3](=O)([O-])O)[C@@H](O)[C@H]1O]

or pharmaceutically acceptable salts or solvates thereof as well as mixtures thereof. AMP can be obtained for example from Sigma Aldrich.

The term “Amplex® Red” or “AR” as used herein means:

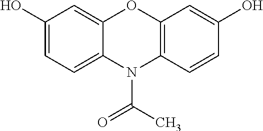 [SMILES: CC(=O)N1c2ccc(O)cc2Oc2cc(O)ccc21]

or pharmaceutically acceptable salts or solvates thereof as well as mixtures thereof. Amplex® Red can be obtained for example from Resazurin which is structurally related and has the formula 7-Hydroxy-3H-phenoxazin-3-one 10-oxide is also referred to as Amplex® Red. Accordingly, Amplex® Red as used herein includes both AR and Resazurin.

The term “5-Bromo-4-chloro-3-indolyl phosphate” or “BCIP” means as used herein a compound having the structure:

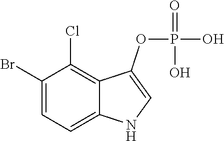 [SMILES: O=P(O)(O)Oc1c[nH]c2ccc(Br)c(Cl)c12]

or pharmaceutically acceptable salts or solvates thereof as well as mixtures thereof. BCIP can be obtained for example from Sigma Aldrich.

The term “ionizable product”, as used herein means a product generated by a reporter enzyme, that comprises one or more ionizable groups. For example, an ionizable product may have one or more basic or amine groups for positive ionization and one or more acidic or hydroxyl groups for negative ionization. Ionizable groups may include ═NH, —NH2, guanidinium, methyl, ethyl, alky, phenyl, ribose, inositiol, phospholipid, carbohydrate, nucleic acid, carbonyl, aldehyde, ketone, carboxyl, hydroxyl, enol, guanidium, imidazole, sulfhydryl, disulfide, sulfate, phosphate, sulfonyl, nitrate, nitric oxide, thioester, ester, ether, anhydride, phosphoryl, mixed anhydride, and/or other ionizable groups known in the art. An ionizable product assessed, optionally efficiently enters the gas phase by electrospray ionization.

The term “L-(+)-2-amino-6-phosphonohexanoic acid” as used herein means:

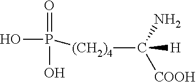

or pharmaceutically acceptable salts or solvates thereof as well as mixtures thereof. L-(+)-2-amino-6-phosphonohexanoic acid can be obtained for example from Sigma Aldrich.

The term “Lumigen® TMA-3” or “TMA-3” as used herein means

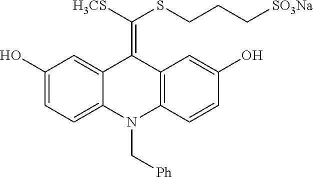

or pharmaceutically acceptable salts or solvates thereof as well as mixtures thereof. TMA-3 can be obtained for example from Beckman Coulter Company.

The term “Lumigen® TMA-6” or “TMA-6” as used herein means

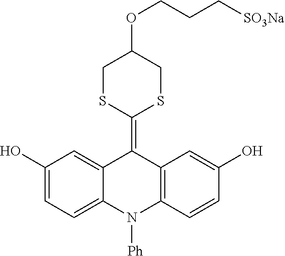

or pharmaceutically acceptable salts or solvates thereof as well as mixtures thereof. TMA-6 can be obtained for example from Beckmann Coulter Company.

The term “4-Methylumbelliferyl phosphate” or “4-MUP” as used herein means a compound having the structure:

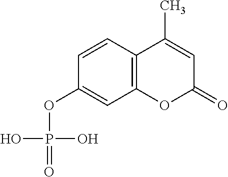 [SMILES: Cc1cc(=O)oc2cc(OP(=O)(O)O)ccc12]

or pharmaceutically acceptable salts or solvates thereof as well as mixtures thereof. 4-MUP can be obtained for example from Sigma Aldrich.

The term “Naphthol ASMX phosphate” as used herein means a compound having the structure:

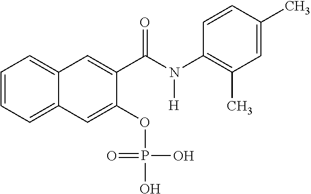 [SMILES: Cc1ccc(NC(=O)c2cc3ccccc3cc2OP(=O)(O)O)c(C)c1]

or pharmaceutically acceptable salts or solvates thereof as well as mixtures thereof. Naphthol ASMX phosphate can be obtained for example from Sigma Aldrich.

The term “O-phospho-DL-Threonine” as used herein means a compound having the structure:

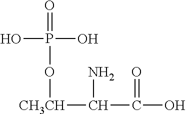

or pharmaceutically acceptable salts or solvates thereof as well as mixtures thereof. O-phospho-DL-Threonine can be obtained for example from Sigma Aldrich.

The term “Para nitrophenol phosphate” or “PNPP” as used herein means a compound having the structure:

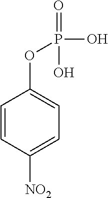 [SMILES: O=[N+]([O-])c1ccc(OP(=O)(O)O)cc1]

or pharmaceutically acceptable salts or solvates thereof as well as mixtures thereof. Para nitrophenol phosphate can be obtained for example from Sigma Aldrich.

The term “phenylbenzene ω phosphono-α-amino acid” as used herein means compound having the structure:

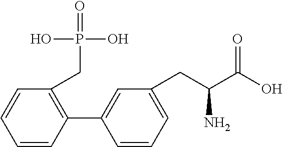 [SMILES: N[C@@H](Cc1cccc(-c2ccccc2CP(=O)(O)O)c1)C(=O)O]

or pharmaceutically acceptable salts or solvates thereof as well as mixtures thereof. Phenylbenzene w phosphono-a-amino acid can be obtained for example from Sigma Aldrich.

The term “pyridoxamine 5-phosphate” or “PA5P” as used herein means compound having the

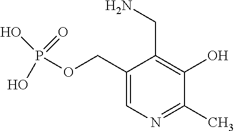 [SMILES: Cc1ncc(COP(=O)(O)O)c(CN)c1O]

or pharmaceutically acceptable salts or solvates thereof as well as mixtures thereof. PA5P can be obtained for example from Sigma Aldrich.

The term “sphingosine-1 phosphate” as used herein means a compound having the structure:

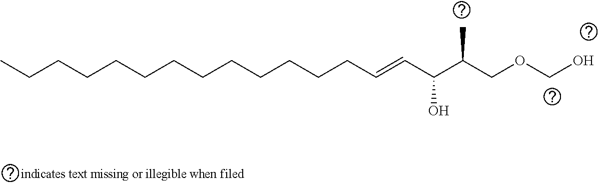 [SMILES: CCCCCCCCCCCCC/C=C/[C@@H](O)[C@@H](C)COCO]

or pharmaceutically acceptable salts or solvates thereof as well as mixtures thereof. Sphingosine-1 phosphate can be obtained for example from Sigma Aldrich.

The term “detection oligonucleotide probe” as used herein comprises a oligonucleotide coupled to a secondary target moiety such as biotin wherein the oligonucleotide or a portion thereof is complementary to and binds selectively to a target nucleic acid molecule, for example, but not limited to, a bacterial, viral or fungal nucleic acid sequence. The detection oligonucleotide probe can be a detection oligonucleotide primer in some embodiments. The detection oligonucleotide probe can also optionally be coupled to the secondary target moiety, such as biotin. The detection oligonucleotide probe can also optionally be coupled to an enzyme such as the reporter enzyme. For example, the detection oligonucleotide can be optionally coupled to enzymes or catalysts including but not limited to ribozyme, a DNAzyme, phosphatase (for example AP), peroxidase (for example HRP), DNA polymerase, or glucose oxidase. For example, the detection oligonucleotide probe can comprise a single stranded oligonucleotide sequence complementary to that of the target nucleic acid molecule and can selectively bind to the target nucleic acid molecule through hybridization.

It can be appreciated by a person skilled in the art that the secondary target moiety and the secondary target binding moiety have high mutual affinity such that the secondary target moiety and the secondary target binding moiety selectively bind to each other. Accordingly, it can be appreciated by a person skilled in the art that a suitable secondary target binding moiety can be selected by a person skilled in the art based on the nature of the secondary target moiety and vice versa. The following list contains non-limiting examples of pairs of selectively binding chemical entities. The secondary target moiety and the secondary target binding moiety can be selected from pairs of chemical entities listed below. For example, the secondary target moiety can be biotin. For example, the secondary target binding moiety can be avidin or streptavidin.

### List of High Affinity Selective Binding Pairs of Chemical Entities:

| SEQ ID NO | Tag | Binding partner |
|---|---|---|
| | Biotin | Avidin |
| | Biotin | Streptavidin |
| 47 | ALFA-tag (SRLEEELRRRLTE) | Single-domain antibodies |
| 48 | AviTag (GLNDIFEAQKIEWHE) | Avidin or Streptavidin |
| | biotinylated | |
| 49 | C-Tag (EPEA) | single-domain camelid antibody |
| 50 | Calmodulin-Tag | Calmodulin |
| | (KRRWKKNFIAVSAANRFKKISSSGAL) | |
| 51 | Polyglutamate tag (EEEEEE) | anion-exchange resin (e.g. Mono-Q) |
| | Polyarginine tag | cation-exchange resin (from 5 to 9 |
| | | consecutive R) |
| 52 | E-tag (GAPVPYPDPLEPR) | Anti-E-tag antibody |
| 53 | FLAG-tag (DYKDDDDK) | Anti-FLAG-tag antibody |
| 54 | HA-Tag (YPYDVPDYA) | Anti-HA-Tag antibody |
| | His-Tag (5-10 histidines) | Nickel or cobalt chelate |
| 55 | Myc-Tag (EQKLISEEDL) | Anti-Myc-Tag antibody |
| 56 | NE-tag (TKENPRSNQEESYDDNES) | Anti-NE-Tag IgG1 antibody |
| 57 | Rho1D4-tag (TETSQVAPA) | Anti-Rho1D4-tag antibody |
| 58 | S-tag (KETAAAKFERQHMDS) | Anti-S-tag antibody |
| 59 | SBP-tag | Streptavidin |
| | (MDEKTTGWRGGHVVEGLAGELEQLR | |
| | ARLEHHPQGQREP) | |
| 60 | Softag 1 (SLAELLNAGLGGS) | Anti-Softag 1 antibody |
| 61 | Softag 3 (TQDPSRVG) | Anti-Softag 3 antibody |
| 62 | Spot-tag (PDRVRAVSHWSS) | Single-domain antibody nanobody |
| 63 | Strep-tag (WSHPQFEK) | Streptavidin |
| 64 | Strep-tag (WSHPQFEK) | Streptactin |
| 65 | T7-tag (MASMTGGQQMG) | Anti-T7-tag antibody |
| 66 | TC-tag (CCPGCC) | FIASH and ReAsH biarsenical |
| | | compounds |
| 67 | Ty1 tag (EVHTNQDPLD) | Anti-Ty1 tag antibody |
| 68 | V5 tag (GKPIPNPLLGLDST) | Anti-V5 tag antibody |
| 69 | VSV-tag (YTDIEMNRLGK) | Anti-VSV tag antibody |
| 70 | Xpress tag (DLYDDDDK) | Anti-Xpress tag antibody |
| 71 | Isopeptag (TDKDMTITFTNKKDAE) | pilin-C protein |
| 72 | SpyTag (AHIVMVDAYKPTK) | SpyCatcher protein |
| 73 | SnoopTag (KLGDIEFIKVNK) | SnoopCatcher protein |
| 74 | DogTag | SnoopTagJr protein |
| | (DIPATYEFTDGKHYITNEPIPPK) | |
| 75 | SdyTag (DPIVMIDNDKPIT) | SdyCatcher protein |
| | Biotin Carboxyl Carrier Protein | Streptavidin |
| | Glutathione-S-transferase tag | Glutathione |
| | Green Fluorescent protein (GFP) tag | GFP-antibody |
| | HaloTag | Haloalkane substrates |
| | SNAP-tag | benzylguanine derivatives |
| | CLIP-tag | benzylcytosine derivatives |
| | HUH-tag | HUH specific DNA sequence |
| | Maltose-binding protein-tag | Amylose agarose |
| | Nus-tag | Nus tag antibody |
| | Thioredoxin-tag | Anti-Thioredoxin-tag antibody |
| | Fc-tag | Protein-A sepharose |
| | CRDSAT-tag (carbohydrate Recognition | Lactose, agarose, sepharose |
| | Domain) | |

The term “oligonucleotide” as used herein as used herein refers to a sequence of nucleoside or nucleotide monomers consisting of naturally occurring bases, sugars and intersugar (backbone) linkages. The term also includes modified or substituted sequences comprising non-naturally occurring monomers or portions thereof. The nucleic acid sequences of the present application may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The nucleic acid can be either double stranded or single stranded, and represents the sense or antisense strand. For example, the capture, detection, target or primer sequences can be oligonucleotides.

The term “reporter enzyme detection probe” as used herein comprises a reporter enzyme component comprising an enzymatic activity, coupled to a detection probe component comprising a secondary target binding moiety, for example avidin or streptavidin when the secondary target moiety is biotin. The reporter enzyme is optionally a peroxidase such as horseradish peroxidase or a phosphatase such as alkaline phosphatase although any stable enzyme that can produce ionizable products can be used including for example a lyase, hydrolase, synthase, synthetase, oxidoreductase, dehydrogenase, oxidase, transferease, isomerase, ligase, protease, such as trypsin, proteinase, peroxidase, glucose oxidase, myeloperoxidase, oxidase, monooxygenase, cytochrome, phosphatase such as alkaline phosphatase, decarboxylase, lipase, caspase, amylase, peptidase, transaminase, and kinase. Additional enzymes can include DNA or RNA polymerase, TAQ, restriction enzymes, klenow fragment, DNA ligase. The secondary target binding moiety selectively binds the secondary target moiety of the detection oligonucleotide probe. For example, the secondary target binding moiety comprises avidin or streptavidin that selectively binds a biotinylated detection oligonucleotide probe (e.g. wherein the secondary target moiety comprises biotin).

The term “selective” as used herein in reference to a probe, optionally an oligonucleotide, is used contextually, to characterize the binding properties of the probe, optionally an oligonucleotide. For example, an oligonucleotide probe that binds selectively to a given target nucleic acid molecule will bind to that target nucleic acid molecule either with greater avidity or with more specificity, relative to another, different target nucleic acid molecule. In an embodiment, the probe, optionally an oligonucleotide probe, binds at least 2 fold, 3 fold, or 5 fold more efficiently, optionally 3-5 fold, 5-7 fold, 7-10, 10-15, 5-15, or 5-30 fold more efficiently.

The term “target nucleic acid molecule” as used herein refers to any nucleic acid polymer that comprises a sequence that is complementary to the oligonucleotide portion of a detection oligonucleotide probe. For example, the target nucleic acid molecule can be RNA or DNA, or derivatives thereof. The target nucleic acid can be any nucleic acid that is at least 30 nucleotides long. For example, the target nucleic acid molecule can be about or at least 30 nucleotides, about or at least 40 nucleotides, about or at least 50 nucleotides, about or at least 80 nucleotides, about or at least 100 nucleotides, about or at least 130 nucleotides, about or at least 180 nucleotides, about 200 nucleotides, about 250 nucleotides, about 300 nucleotides, about 350 nucleotides, about 450 nucleotides, about 600 nucleotides, about 700 nucleotides, about 850 nucleotides, or about 1000 nucleotides. In some embodiments, the target nucleic acid molecule is about 30 nucleotides to about 1500 nucleotides in length. For example, the target nucleic acid molecule is about 30 nucleotides to about 1000 nucleotides in length, about 30 nucleotides to about 300 nucleotides in length, about 100 nucleotides to about 500 nucleotides in length, about 100 nucleotides to about 600 nucleotides in length, about 100 nucleotides to about 700 nucleotides in length, about 100 nucleotides to about 800 nucleotides in length, about 100 nucleotides to about 900 nucleotides in length, or about 100 nucleotides to about 1000 nucleotides in length. For example, the target nucleic acid molecule can be single stranded or double stranded. For example, the target nucleic acid molecule can be plasmid DNA, a bacterial, viral, or fungal nucleic acid molecule or a mammalian or plant nucleic acid e.g. in a gene or in mRNA. The target nucleic acid can also be a synthetic nucleic acid for detection of nucleic acid tagged compounds and the like.

### II. Methods and Kits

Described herein is a transformative technology that permits detection of nucleic acid molecules in the femto mol to pico mol ranges and/or lower. It is demonstrated herein that detection in the zepto mol to atto mol range can be achieved.

Enzyme linked immuno sorbent assays (ELISA) are the preferred analytical method for the repetitive quantitative analysis of polypeptides molecules of biomedical importance: ELISA may use reporter enzymes such as Horseradish peroxidase (HRP) and or alkaline phosphatase (AP) coupled to specific detection antibodies that capture and bind to each analyte of importance (Engvall, 1971; Van Weemen 1971).

At present substrates for the reporter enzymes horseradish peroxidase (HRP) or alkaline phosphatase (AP) yield colored, fluorescent or luminescent products. The present disclosure provides a method for detecting the enzymatic products of reporter enzymes that ionize efficiently with a high signal to noise ratio measured by mass spectrometry. Mass spectrometry is sensitive enough to permit detections at amounts far below ECL, fluorescence or colorimetric methods, but also permits monitoring of multiple substrates and products at discrete m/z values. It is possible using the methods described herein to measure the products of common industrial reporter enzymes to zepto mol amounts or lower with limits of quantification to atto mol amounts or lower.

The use of mass spectrometry to measure small molecules may commonly reach the femto to pico mol levels with high signal to noise. The industrial enzymes HRP or AP for example are rugged and durable and have a high catalysis rate for the creation of new small molecule products. The AP or HRP enzymes are for example covalently attached to a specific detection probe such as a polypeptide or antibody that may bind their target and then catalyze many different product reactions over the course of a brief incubation. Thus, the binding of atto mol, or even sub atto mol, amounts of enzyme-probe will yield amounts of small molecule products that accumulate in the femto mol to pico mol range well within the detectable range of by LC-ESI-MS/MS.

Liquid chromatography electrospray ionization and tandem mass spectrometry (LC-ESI-MS/MS) is more sensitive than colorimetric, fluorescent or ECL detection. The combination of the enzymatic production of reported molecules coupled with sensitive mass spectrometry for highly ionizable substrates should provide sensitivity in excess of RIA but without the requirement for standards labelled with isotope or probes labeled with isotope.

Quantification of HRP and AP is demonstrated using LC-ESI-MS/MS to detect the products of the AP and HRP reporter enzyme reactions. It is demonstrated herein that a mass spectrometer can also detect the small molecule products of reporter enzyme activity bound to a specific molecular probe such as an antibody. One atto mol or less of a reporter enzyme such as AP or HRP bound to a specific molecular probe such as a detection antibody will rapidly form femto mol to pico mol amounts of reporter enzyme reaction products well within the reliable detection and quantification limits of LC-ESI-MS/MS. Hence in ELiMSA and related DNA methods (e.g. DNA ELiMSA) the reporter enzymes such as HRP or AP may produce a range of products that can be easily distinguished and detected by mass spectrometry. Antibodies coupled to reporter enzymes that are widely used in biomedical and environmental applications can now be detected and quantified using very sensitive mass spectrometry to create a sensitive and flexible system. Since mass spectrometers can separate and analyze many analytes simultaneously using the methods described herein can allow identification and quantification of many different antigens at the same time to levels far below that which is possible by direct mass spectrometric analysis.

The reaction is reporter enzyme dependent. For example, it is demonstrated herein that incubating a substrate that can be acted upon by the reporter enzyme detection probe in an appropriate substrate reaction solution produces little or no signal in the absence of the reporter enzyme detection probe. In contrast, the addition of reporter enzyme detection probe comprising HRP or AP enzyme resulted in strong detection of an ELiMSA product ion. The product ion was shown to be dependent on the presence of the enzyme, and to be both time and concentration dependent. Thus, the ELiMSA product ions show all the hallmarks of an enzyme dependent assay.

Depending on the reporter enzyme or enzyme substrate, different ionizable products can be detected. Fragments thereof can also be detected. For example, adenosine can be ionized and detected at 268 m/z or fragmented and the fragment can be detected at 136 m/z.

As shown in the examples, a capture oligonucleotide probe can be used to capture a target nucleic acid molecule. In other examples the target nucleic acid molecule can be attached, covalently or non-covalently, to a solid support (e.g. solid phase) directly and a labelled detection probe optionally a labelled primer, can be used to detect the attached target nucleic acid molecule.

In one aspect, the present disclosure includes a method of detecting a target nucleic acid molecule comprising
a.
   i. incubating a sample putatively comprising the target nucleic acid molecule with a capture oligonucleotide probe that comprises a sequence complementary to the target nucleic acid molecule and that is attached to a solid phase, in a first binding solution, optionally wherein the solid phase is attached to the capture oligonucleotide probe through a linker; or
   ii. incubating a sample putatively comprising the target nucleic acid molecule with a solid phase to attach said sample/target nucleic acid molecule to said solid phase, in a first binding solution, optionally wherein the solid phase is attached to the sample/target nucleic acid molecule through a linker;
b. binding any target nucleic acid molecule to a detection oligonucleotide probe in a second binding solution under conditions for forming a target:detection complex;
c. incubating any target:detection complex with a reporter enzyme detection probe in a third binding solution under conditions for forming a target:detection:enzyme complex;
d. washing the solid phase to remove any unbound reporter enzyme detection probe with a washing solution;
e. incubating any target:detection:enzyme complex with a reporter enzyme detection probe substrate in a substrate reaction solution to generate one or more ionizable products; and
f. detecting at least one of the one or more ionizable products using mass spectrometry (MS),
wherein
   i. at least the third binding solution among the first binding solution, the second binding solution, and the third binding solution is substantially free of inorganic salt;
   ii. the washing solution is substantially free of inorganic salt;
   iii. the method further comprises cross-linking components of any target:detection:enzyme complex and the capture oligonucleotide probe prior to the optional step d) and the step e); and/or
   iv. the method further comprises separating the one or more ionizable products priorto detection using MS; and
wherein detection of the at least one of the one or more ionizable products is indicative of the sample comprising the target nucleic acid molecule.

The detection oligonucleotide probe can be a detection oligonucleotide primer. In such cases, the step comprises amplifying the target nucleic acid molecule with a detection oligonucleotide primer, in an amplification solution and binding any amplified target to the detection oligonucleotide probe in the second binding solution under conditions for forming a target:detection complex.

It is also contemplated that the detection oligonucleotide probe can be covalently attached to the reporter enzyme directly through covalent attachment, optionally though a linker. In such a case, the target:detection complex is sufficient to react with the reporter enzyme detection probe substrate. Thus, the secondary target moiety and the secondary target binding moiety are not required. Accordingly, in another aspect, the present disclosure includes a method of detecting a target nucleic acid molecule comprising
a.
   i. incubating a sample putatively comprising the target nucleic acid molecule with a capture oligonucleotide probe that comprises a sequence complementary to the target nucleic acid molecule and that is attached to a solid phase, in a first binding solution, optionally wherein the solid phase is attached to the capture oligonucleotide probe through a linker; or
   ii. incubating a sample putatively comprising the target nucleic acid molecule with a solid phase to attach said sample/target nucleic acid molecule to said solid phase, in a first binding solution, optionally wherein the solid phase is attached to the sample/target nucleic acid molecule through a linker;
b. binding any target nucleic acid molecule to a detection oligonucleotide probe in a second binding solution under conditions for forming a target:detection complex, the detection oligonucleotide probe comprising an oligonucleotide and a reporter enzyme;
c. washing the solid phase to remove any unbound detection oligonucleotide probe with a washing solution;
d. incubating the target:detection complex with a reporter enzyme detection probe substrate in a substrate reaction solution to generate one or more ionizable products; and
e. detecting one or more of the one or more ionizable products using mass spectrometry (MS),
wherein either
   i. at least the second binding solution among the first binding solution, and the second binding solution is substantially free of inorganic salt;
   ii. the washing solution is substantially free of inorganic salt;
   iii. the method further comprises cross-linking components of any target:detection complex and the capture oligonucleotide probe prior to the optional step c) and the step d); and/or
   iv. the method further comprises separating the one or more ionizable products prior to detection using MS; and
wherein detection of the at least one of the one or more ionizable products is indicative of the sample comprising the target nucleic acid molecule.

The detection oligonucleotide probe can be a detection oligonucleotide primer. In such cases, the step comprises amplifying the target nucleic acid molecule with a detection oligonucleotide primer, in an amplification solution and binding any amplified target to the detection oligonucleotide probe in the second binding solution under conditions for forming a target:detection complex.

In some embodiments, the second binding solution, the third binding solution and the substrate reaction solution each comprises a Tris buffer.

In some embodiments, the capture oligonucleotide probe is directly immobilized to the solid phase, optionally by non-covalent or covalent binding to the solid phase.

In some embodiments, the capture oligonucleotide probe comprises a oligonucleotide that has a sequence complementary to a part of the target nucleic acid molecule that is at least 25 nucleotides in length, at least 35 nucleotides in length, optionally the capture oligonucleotide probe has a sequence complementary to a part of the sequence of the target nucleic acid molecule that is about 30 nucleotides to about 60 nucleotides in length, or about 40 nucleotides to about 55 nucleotides in length.

In some embodiments, the detection oligonucleotide probe comprises an oligonucleotide that has a sequence complementary to another part of the target nucleic acid molecule, and a secondary target moiety selected from biotin.

In some embodiments, the sequence of the oligonucleotide of the detection oligonucleotide probe complementary to the other part of the sequence of the target nucleic acid molecule is at least 25 nucleotides in length, at least 35 nucleotides in length, optionally the detection oligonucleotide probe is about 30 nucleotides to about 60 nucleotides in length, or about 40 nucleotides to about 55 nucleotides in length.

In some embodiments, the capture oligonucleotide probe and the detection oligonucleotide probe can both bind the target nucleic acid molecule at non-overlapping regions, optionally the non-overlapping regions are directly adjacent, optionally the non-overlapping regions are at least one nucleotide apart, optionally the non-overlapping regions are at least 5 nucleotides apart, optionally the non-overlapping regions are about 2 nucleotides, about 5 nucleotides, about 10 nucleotides, about 20 nucleotides, about 25 nucleotides, about 50 nucleotides, about 100 nucleotides, about 500 nucleotides, or about 1000 nucleotides apart. In some embodiments, the non-overlapping regions are about 1 kb apart. In some embodiments, the non-overlapping regions are more than 1 kb apart.

In some embodiments, when a binding solution and/or a washing solution is substantially free of inorganic salt, the binding solution and/or the washing solution is each independently a volatile solution. In some embodiments, the volatile solution comprises a volatile buffer. In some embodiments, the volatile buffer is selected from ethanolamine, ammonium bicarbonate, ammonium formate, pyridinium formate, trialkylammonium/formic acid, ammonium acetate, trialkylammonium bicarbonate, N-ethylmorpholine/acetate, trialkylammonium acetate, or combinations thereof. In some embodiments, the volatile buffer is selected from ethanolamine, ammonium acetate, trialkylammonium bicarbonate, or combinations thereof. In some embodiments, the trialkylammonium is selected from trimethylammonium, triethylammonium, or combinations thereof. In some embodiments, the volatile buffer is ethanolamine. It can be appreciated by a person skilled in the art that ammonium bicarbonate is not stable to heat. For example, ammonium bicarbonate decomposes at about or above 90° C. Accordingly, for steps involving heating, other volatile buffers such as ethanolamine is preferred.

In some embodiments, when the first binding solution, the second solution, the third binding solution, and/or the washing solution is substantially free of inorganic salt, the first binding solution, the second solution, the third binding solution, and/or the washing solution each independently comprises ethanolamine, optionally the second binding solution and the third binding solution each comprises ethanolamine, optionally the first binding solution, the second binding solution, and the third binding solution each comprises ethanolamine, optionally the washing solution comprises ethanolamine.

In some embodiments, step a) and step b) are performed simultaneously, and the first binding solution of step a) is the second binding solution of step b).

In some embodiments, the first binding solution, the second binding solution, the third binding solution, and the substrate reaction solution each independently has a pH of about 7 to about 10, optionally of about 7 to about 8, optionally about 8.8.

In some embodiments, any of the volatile binding solutions can be used to wash the solid support, optionally to remove any inorganic salt that may be present.

In some embodiments, the target:detection:enzyme complex is incubated with the reporter enzyme detection probe substrate in the substrate reaction solution to generate the one or more ionizable products for a period of time less than 72 hours, less than 24 hours, less than 12 hours, less than 60 minutes, less than 50 minutes, less than 40 minutes, less than 30 minutes, less than 20 minutes, less than 15 min, less than 10 min, less than 5 min, less than 2 min, or less than 1 min.

In some embodiments, at least the third binding solution among the first binding solution, the second binding solution, and the third binding solution is substantially free of inorganic salt and comprises a volatile buffer described herein.

In some embodiments, the method comprises washing the solid phase to remove any unbound reporter enzyme detection probe with the washing solution, wherein the washing solution is substantially free of inorganic salt and comprises a volatile buffer as described herein.

In some embodiments, the components of any target:detection:enzyme complex and the capture oligonucleotide probe are cross-linked prior to the optional step d) and the step e), and the cross-linking is through H-hydroxysuccinimide (NHS), N-oxysuccinimide (NOS), maleimide, hydrazide, glutaraldehyde coupling, disuccinimidyl suberate (DSS) cross-linking or PEG crosslinking.

In some embodiments, the cross-linking of the components of any target:detection:enzyme complex and the capture oligonucleotide probe is through glutaraldehyde coupling, DSS cross-linking, or PEG cross-linking.

In another aspect, the present disclosure includes a method of quantifying the amount of a target nucleic acid molecule in a sample comprising the steps:
a. detecting a target nucleic acid molecule according to a method of the present disclosure; and
b. quantifying the amount of target nucleic acid molecule in the sample based on the intensity of the signal for one or more of the ionizable products detected by mass spectrometry.

In some embodiments, the quantification comprises comparing the intensity of the signal for one or more products against signal intensities generated using known quantities of target substance, under similar conditions.

In some embodiments, the target nucleic acid molecule is present or suspected to be present in the sample in or up to a pico mol, femto mol, or atto mol range.

In some embodiments, the target nucleic acid molecule is selected from DNA, RNA, and combinations and derivatives thereof.

In some embodiments, the sample is a biological sample, industrial product, environmental sample, or a polymerase chain reaction (PCR) reaction product. In some embodiments, the biological sample is a blood sample, urine sample, fecal sample, effusate, tissue sample or sputum sample.

In another aspect, the present disclosure includes a method of detecting a target nucleic acid molecule comprising
performing a nucleic acid amplification such as a polymerase chain reaction (PCR) or a hybridization chain reaction (HCR) or rolling circle reaction or other nucleic acid reaction on a test sample putatively comprising the target nucleic acid molecule with a modified primer and a second primer to obtain an amplified nucleic acid product, optionally a PCR product, comprising the modified primer, the modified primer being functionalized with a secondary target moiety or a reporter enzyme;
separating the amplified nucleic acid product from any unreacted modified primer;
when the modified primer is functionalized with the secondary target moiety, incubating the amplified nucleic acid product with a reporter enzyme detection probe in a first binding solution under conditions to form an amplified nucleic acid product:reporter enzyme complex, and removing any unbound reporter enzyme detection probe with a washing solution, the reporter enzyme detection probe comprising a secondary target binding moiety and a reporter enzyme;
incubating the amplified nucleic acid product or the amplified nucleic acid product:reporter enzyme complex with a reporter enzyme substrate in a substrate reaction solution to generate one or more ionizable products; and
detecting the one or more ionizable products using mass spectrometry (MS),
wherein when the modified primer is a forward primer, the second primer is a reverse primer, and
wherein when the modified primer is a reverse primer, the second primer is a forward primer.

In some embodiments, the second primer is attached to a solid phase, optionally the second primer is attached to the solid phase through a linker.

In some embodiments, the second primer is directly attached to the solid phase, optionally by non-covalent or covalent binding to the solid phase.

In some embodiments, the separation of the unreacted modified primer from the amplified nucleic acid product is by centrifugation, filtration and/or solvent wash.

In some embodiments, the method further comprises incubating the amplified nucleic acid product comprising the modified primer with a solid phase in a second binding solution under conditions to bind the amplified nucleic acid product onto the solid phase, prior to incubating the amplified nucleic acid product with the reporter enzyme detection probe, the solid phase having a capture oligonucleotide probe attached thereon that comprises a sequence complementary to the amplified nucleic acid product, optionally, the solid phase is attached to the capture oligonucleotide probe through a linker.

In some embodiments, the capture oligonucleotide probe is directly attached to the solid phase, optionally by non-covalent or covalent binding to the solid phase.

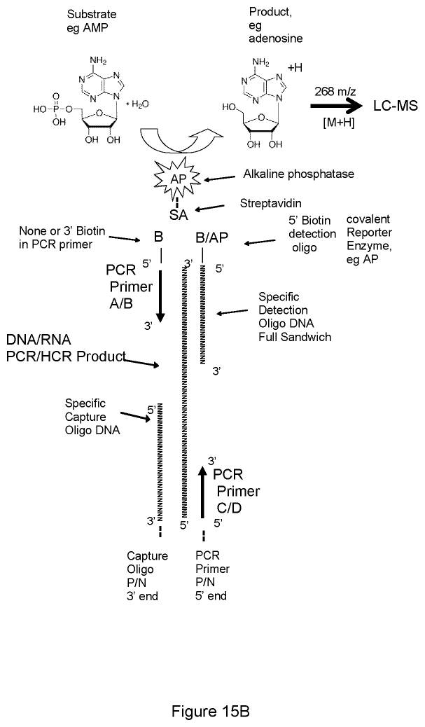

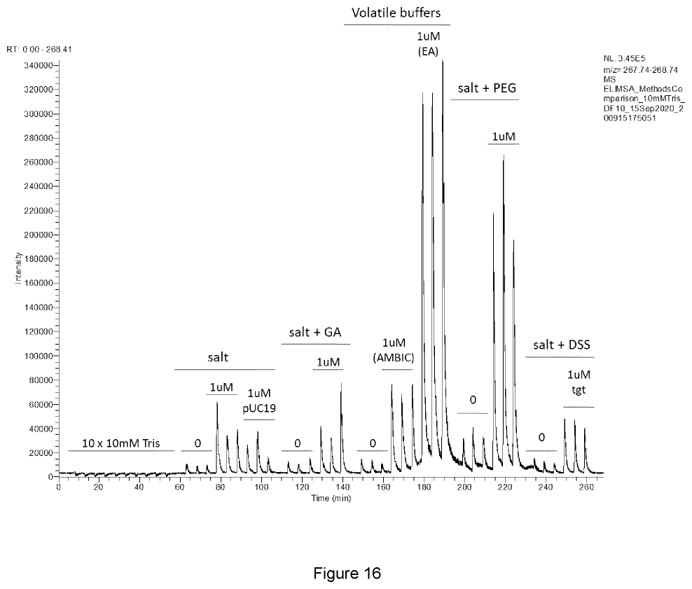

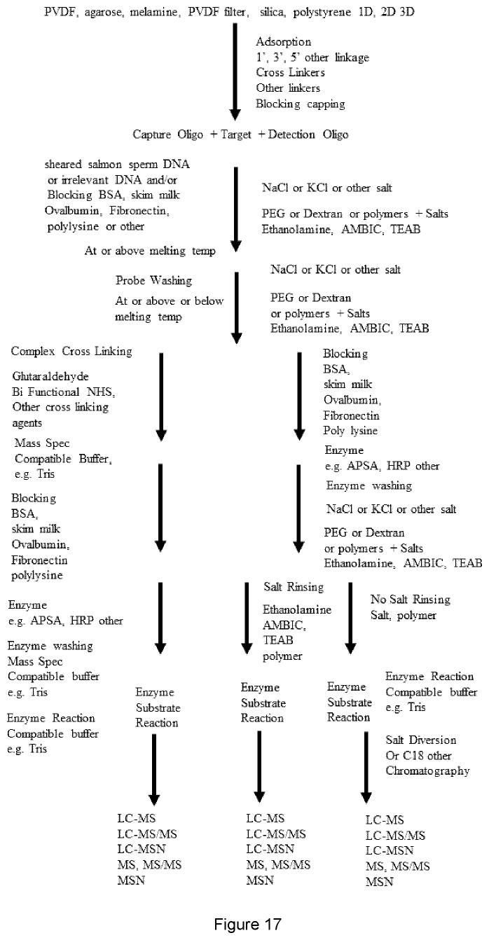

For example, various embodiments are shown in FIG. 15A and FIG. 15B. FIGS. 15 A and B show embodiments that can be referred to as “full sandwich”, or “half sandwich” involving in some embodiments amplification, for example producing a PCR product, and embodiments of covalent or chemical linkage or non-covalent attachment such as adsorption to a solid support. For specifically, “half-sandwich” embodiment is shown using biotinylated primers and optionally a 5′ attachment or a 3′ attachment of the capture oligonucleotide probe, for example using a NOS chemical attachment plate. Also shown is an embodiment, wherein the target nucleic acid molecule is adsorbed to PVDF and detected with a biotinylated detector probe. Also shown are “full sandwich” embodiments, where a capture oligonucleotide probe and a detection oligonucleotide probe are used. The target nucleic acid sample can be chemically attached or adsorbed and detected using a tagged detector probe. Other embodiments and combinations are also described herein.

The “R” shown in FIG. 15A can be any one or more of an amine or other linker, biotin or other tag, or attachment to a solid support. For example, the amine may be an amine group present in an oligonucleotide or added to the oligonucleotide.

The linker may be a chemical bond or may for example include a moiety such as a PEG chain that ends in amine. Other moieties such as a a carbon chain that comprises an amine.

The linker can be an amine (or amine linkage once linked), or NHS, or carboxyl link or cysteine link or a PEG for example with an amine or amine reactive group or any other suitable link. Others can be used including others that are described herein or in the table below.

| Reactivity class | Chemical group |
|---|---|
| Carboxyl-to-amine reactive groups | Carbodiimide (e.g., EDC) |
| Amine-reactive groups | NHS ester |
| | Imidoester |
| | Pentafluorophenyl ester |
| | Hydroxymethyl phosphine |
| Sulfhydryl-reactive groups | Maleimide |
| | Haloacetyl (Bromo- or Iodo-) |
| | Pyridyldisulfide |
| | Thiosulfonate |
| | Vinylsulfone |
| Aldehyde-reactive groups | Hydrazide |
| i.e., oxidized sugars (carbonyls) | Alkoxyamine |
| Photoreactive groups | Diazirine |
| i.e., nonselective, random insertion | Aryl Azide |
| Hydroxyl (nonaqueous)-reactive groups | Isocyanate |

Various tags can be used. For example the tag may be biotin, ALFA-tag, AviTag, C-tag, Calmoudulin-Tag, Polyglutamate Tag, E-Tag, Flag-tag, HA-tag, His-Tag, myc-Tag, NE-tag, Rho1D4-Tag, S-Tag, SBP-Tag, Softag 1, Softag 3, Spot-tag, Strept-tag, T7-tag, TC-tag, Ty1 tag, V5 tag, VSV-tag, Xpress tag, Isopeptag, SpyTag, SnoopTag, DogTag, Sdy Tag, Biotin carboxyl carrier protein, glutathione-S-transferase tas, GFP tag, HaloTag, SNAP-tag, CLIP-tag, HUH-Tag, Maltose-binding protein tag, Nus-tag, thioredoxin-tag, Fc-tag, or CRDSAT-tag. Others for example described elsewhere herein can also be used. The tag is some embodiments is biotin.

As discussed covalent and non covalent attachments can be used. For example, attachment to the support, can be a covalent attachment such as [H-hydroxysuccinimide (NHS), N-oxysuccinimide (NOS), maleimide, hydrazide, glutaraldehyde coupling, or PEG cross-linking or a non-covalent attachment [Adsorption to PVDF, silica, polystyrene, nylon, etc. This may be effected through or without a linker. Such as adsorption to PVDF, polystyrene or silica or nylon, acrylamide, alginate, melamine or any of the support

The solid support can for example be a plate such as a polystyrene plate, or chemically reactive NOS polystyrene plate, and the plate may be a 96 well plate, micro well or nanowell plate, a membrane such as PVDF membrane in for example a 96 well plate, or a micro or nanosized particle such as a bead. Other attachments include for example silica, PVDF, polystyrene, nylon, acrylamide, alginate, melamine

A more specific example is shown in FIG. 15B. In this Figure, P refers to phosphate and N refers to amine. Primer A/B refers to the primer with and without a tag such as biotin. Primer C/D refers to the primer free or affixed to a surface support.

In some embodiments, the capture oligonucleotide probe has a sequence complementary to a part of the sequence of the amplified nucleic acid product comprising the modified primer.

In some embodiments, the first binding solution and/or the washing solution is volatile and substantially free of NaCl.

In some embodiments, the second binding solution being volatile and substantially free of NaCl.

In some embodiments, the first binding solution or the second binding solution each comprises a volatile buffer.

In some embodiments, the volatile buffer is selected from ethanolamine, ammonium bicarbonate, ammonium formate, pyridinium formate, trialkylammonium/formic acid, ammonium acetate, trialkylammonium bicarbonate, N-ethylmorpholine/acetate, trialkylammonium acetate, and combinations thereof.

In some embodiments, the volatile buffer is selected from ethanolamine, ammonium acetate, trialkylammonium bicarbonate, and combinations thereof.

In some embodiments, the trialkylammonium is selected from trimethylammonium, triethylammonium, and combinations thereof.

In some embodiments, the volatile buffer is ethanolamine.

In some embodiments, the method further comprising washing the solid phase with a blocking agent, optionally bovine serum albumin (BSA), prior to binding the amplified nucleic acid product to the solid phase.

In some embodiments, the first binding solution or the second binding solution each independently has a pH of about 7 to about 10, optionally of about 7 to about 8, optionally about 8.8.

In some embodiments, the removing of any unbound reporter enzyme detection probe from the amplified nucleic acid product:reporter enzyme complex is by centrifugation, filtration and/or solvent wash.

In some embodiments, the amplified nucleic acid product or the amplified nucleic acid product:reporter enzyme complex is incubated with the reporter enzyme substrate in the substrate reaction solution to generate the one or more ionizable products for a period of time less than 72 hours, less than 24 hours, less than 12 hours, less than 60 minutes, less than 50 minutes, less than 40 minutes, less than 30 minutes, less than 20 minutes, less than 15 min, less than 10 min, less than 5 min, less than 2 min, or less than 1 min.

In some embodiments, the test sample is a biological sample, industrial product, or environmental sample.

In some embodiments, the biological sample is a blood sample, urine sample, fecal sample, effusate, tissue sample or sputum sample.

In some embodiments, the PCR is selected from real time PCR (rtPCR), quantitative PCR (qPCR), reverse transcription PCR, nested PCR, hybridization chain reaction, rolling circle PCR, and substrate recycling reaction.

The reporter enzyme detection probe can comprise a reporter enzyme component and a detection probe component that are coupled together, optionally covalently. It is also contemplated that in some embodiments, the detection oligonucleotide probe can be attached to the report enzyme directly through covalent attachment optionally through a linker. When the detection oligonucleotide probe is already attached to the reporter enzyme, the reporter enzyme detection probe is not required. In an embodiment, the reporter enzyme comprises peroxidase activity, monooxygenase activity, phosphatase activity, glucose oxidase, protease or caspase activity, for example the reporter enzyme is a peroxidase, monooxygenase, phosphatase, glucose oxidase, protease, endoproteinase, exopeptidase or a caspase. In another embodiment, the reporter enzyme is selected from a lyase, hydrolase, synthase, synthetase, oxidoreductase, dehydrogenase, oxidase, transferase, isomerase, ligase, protease, such as trypsin, endoproteinase, exopeptidase, proteinase, peroxidase, glucose oxidase, myeloperoxidase, oxidase, monooxygenase, cytochrome, phosphatase sicj as alkaline phosphatase, decarboxylase, lipase, caspase, amylase, peptidase, transaminase, and kinase. Additional enzymes can include DNA or RNA polymerase, TAQ, restriction enzymes, klenow fragment, DNA ligase. In yet another embodiment, the reporter enzyme is selected from HRP, AP, ligase, DNA Polymerase (for example klenow or TAQ), restriction enzymes, and proteases, cytochrome monooxygenases, glucose oxidase, GAPDH, and other glycolysis and TCA cycle enzymes.

The solid phase can be any reaction vessel, optionally a bead, rod or plate, such as a microtitre plate, for example having a polystyrene surface. The solid phase may be any surface, including metal, gold, stainless steel, plastic, glass, silica, normal phase, reverse phase, polycarbonate, polyester, PVDF, nitrocellulose, cellulose, poly styrene, polymer, iron, magnetic, coated magnetic, microbeads, nanobeads, nanotubules, nanofibers or fullerene. An immunosorbent polystyrene rod with eight to 12 protruding cylinders has been described for example in U.S. Pat. No. 7,510,687.

The binding of the target nucleic acid molecule to the detection oligonucleotide probe, and of the detection oligonucleotide probe to the reporter enzyme detection probe can occur in a buffered solution. The conversion of the substrate by the reporter enzyme detection probe can occur in a substrate reaction buffer. Suitable buffers include volatile buffers that are substantially free of NaCl and are volatile buffers that are compatible with mass spectrometric conditions. Such suitable buffers include but are not limited to ammonium bicarbonate, ammonium formate, pyridinium formate, trimethylamine/formic acid, ammonium acetate, trimethylamine bicarbonate, N-ethylmorpholine/acetate, triethylamine/formic acid, triethylamine bicarbonate, or a polymer such as polyethylene glycol or dextran sulfate and combinations thereof. Buffers that hold the pH of the solution near the optimal for the maximal activity of the reporter enzyme are preferred. These same buffers might be used for the binding of the test substance or the reaction buffer.

The same binding buffer may be used for the binding of the target nucleic acid molecule to the detection oligonucleotide probe, and of the detection oligonucleotide probe to the reporter enzyme detection probe. Optionally, the substrate reaction buffer may be the same as the binding buffer.

In embodiments comprising an amplification, the binding buffer may comprise reagents for amplification and be referred to as an amplification solution, e.g. comprising polymerase, nucleotides, etc. in a buffer suitable for amplification.

The method disclosed herein can also be performed in solution in the absence of a solid phase, wherein the target substance is not immobilized but suspended on microbeads or magnetic microbeads or in a colloidal suspension or otherwise not entirely immobilized but free to move in a solution

Substrates that produce ionizable products that provide a high signal to noise ratio are desired. For example, the selected signal to noise ratio is at least 3, at least 4, at least 5, at least 6, at least 10. In an embodiment, the signal to noise ratio is greater than or equal to 5. The signal to noise ratio is the ratio of the mass signal (peak height) to noise (amplitude of base level fluctuation). The signal to noise ratio can be determined for example, by measuring the ratio of signal intensity from a blank sample or base line compared to that of a known quantity of analyte or a sample using MS. An example of a substrate that produces an ionizable product that when ionized to a product ion has a high signal to noise ratio is naphthol ASMX phosphate, which is dephosphorylated. A high signal to noise ratio, as used herein, is a signal to noise ratio greater than at least 5, at least 6, at least 10.

The substrate requires at least one ionizable group for example comprising at least one of NO₂, SO₄, PO₃, NH₂, ═NH—, COOH, NH—NHR—, NH₂—NR—NH₂, ionizable for example by electrospray or MALDI, and is a substrate for a selected reporter enzyme. In the case of HRP for example, a suitable substrate is one that is able to donate an electron to H₂O₂. As another example, in the case of phosphatases such as AP the substrate has at least one phosphate group that may be cleaved by the enzyme.

In some embodiments, the methods of the present disclosure further comprise separating the one or more ionizable products prior to detection using MS. In some embodiments, the separation is by liquid chromatograph, centrifugation, filtration, solvent wash, and/or salt diversion. In some embodiments, separation is by liquid chromatography, optionally isocratic normal phase chromatography. In some embodiments, the liquid chromatography is by reverse-phase chromatography. In some embodiments, the reverse-phase chromatography is C18 chromatography. In some embodiments, the liquid chromatography is high-performance liquid chromatography (HPLC). In some embodiments, the HPLC is nanoflow liquid chromatography.

In some embodiments, the step of detecting the one or more ionizable products using MS comprises ionizing the one or more ionizable products, optionally by electrospray ionization (ESI), MALDI, chemical ionization, electron impact, laser desorption, electrical ionization, or heat ionization to produce one or more product ions, and subjecting the one or more product ions to MS optionally tandem MS (MS/MS).

In some embodiments, the ionizing is positive ionization or negative ionization.

In some embodiments, the produced one or more product ions have a selected signal to noise ratio that is at least 3, at least 4, at least 5, at least 6, at least 7, at least 8, at least 9 or at least 10.

In some embodiments, the MS is selected from electrospray ionization tandem MS (ESI-MS/MS), matrix-assisted laser desorption/ionization time-of-flight (MALDI-TOF), tandem MS (MS/MS), multiple rounds of fragmentation MSN, MALDI, electrospray, nanospray, surface ionization, laser desorption & ionization, atmospheric ionization, vacuum ionization, and MS equipped with capillary electrophoresis, ultra sonic or sonic or vibration, nanodroplet or mivrodroplet sample introduction system.

In some embodiments, the detecting using MS comprises recording product ion intensity by single ion monitoring (SIM) and/or product ion parent to fragment transition by single reagent monitoring (SRM).

In some embodiments, the reporter enzyme detection probe comprises a reporter enzyme and optionally a secondary target binding moiety, and wherein the secondary target binding moiety is covalently bound to the reporter enzyme.

In some embodiments, the secondary target moiety is selected from biotin, ALFA-tag, AviTag, C-tag, Calmoudulin-Tag, Polyglutamate Tag, E-Tag, Flag-tag, HA-tag, His-Tag, myc-Tag, NE-tag, Rho1D4-Tag, S-Tag, SBP-Tag, Softag 1, Softag 3, Spot-tag, Strept-tag, T7-tag, TC-tag, Ty1 tag, V5 tag, VSV-tag, Xpress tag, Isopeptag, SpyTag, SnoopTag, DogTag, Sdy Tag, Biotin carboxyl carrier protein, glutathione-S-transferase tas, GFP tag, HaloTag, SNAP-tag, CLIP-tag, HUH-Tag, Maltose-binding protein tag, Nus-tag, thioredoxin-tag, Fc-tag, and CRDSAT-tag, optionally the second target moiety is biotin.

In some embodiments, the secondary target binding moiety binds the secondary target moiety and is selected from avidin, streptavidin, calmodulin, anion-exchange resin, Mono-Q, cation-exchange resin, anti-E-tag antibody, anti-FLAG-tag antibody, anti-HA-tag antibody, nickel or cobalt chelate, anti-Myc-tag antibody, anti-NE-tag antibody, anti-Rho1 D4-tag antibody, anti-S-tag antibody, anti-Softag 1 antibody, anti-Softag 3 antibody, nanobody, streptactin, anti-T7-tag antibody, FIAsH biarsenical compounds, ReAsH biarsenical compounds, anti-Ty1 tag antibody, anti-V5 tag antibody, anti-VSV tag antibody, anti-Xpress tag antibody, pilin-C protein, SpyCatcher protein, SnoopCatcher protein, SnoopTagJr protein, SdyCatcher protein, glutathione, GFP-antibody, haloalkane substrate, benzylguanine derivatives, benzylcytosine derivatives, HUH specific DNA sequence, amylose agarose, Nus-tag antibody, anti-thioredoxin-tag antibody, protein-A sepharose, lactose, agarose, and sepharose, optionally the secondary target binding moiety is selected from avidin and streptavidin.

In some embodiments, the secondary target binding moiety binds the secondary target moiety of the detection oligonucleotide probe and is selected from avidin, and streptavidin when the secondary target moiety is biotin.

In some embodiments, the reporter enzyme is selected from a phosphatase, optionally alkaline phosphatase, lyase, hydrolase, synthase, synthetase, oxidoreductase, dehydrogenase, oxidase, transferease, isomerase, ligase, protease, such as trypsin, proteinase, peroxidase, glucose oxidase, myeloperoxidase, oxidase, monooxygenase, cytochrome, decarboxylase, lipase, caspase, amylase, peptidase, transaminase, kinase activity, DNA or RNA polymerase, optionally TAQ, restriction enzyme, klenow fragment, and DNA ligase.

In some embodiments, the reporter enzyme is selected from alkaline phosphatase, horseradish peroxidase, trypsin, cytochrome C monooxygenase, and myeloperoxidase, optionally, the reporter enzyme is alkaline phosphatase or horseradish peroxidase.

In some embodiments, the one or more ionizable products are readily ionizable under ESI-MS/MS or MALDI-TOF and generates a product ion characterized by a high signal to noise ratio, and the substrate is optionally selected from:
a. a phosphorylated nucleoside, optionally AMP or CMP, or nucleotide, optionally ATP or CTP, phosphorylated alkaloid, phosphorylated amino acid, phosphorylated amino acid polymer, and phosphorylated metabolite when the enzyme is alkaline phosphatase (AP);
b. a compound selected from phenols, amines, optionally phenolic amines, aromatic compounds, olefin halogenations, luminol, pyrogallol, 2,2′-azino-bis(3-ethylbenzothiazoline-6-sulphonic acid (ABTS), and Amplex® Red when the reporter enzyme is horseradish peroxidase (HRP); or from
c. opiates, detergents, dye precursor, alcohols, and matrix.

In some embodiments, the reporter enzyme detection probe substrate is se-lected from pyridoxamine-5-phosphate (PA5P), p-nitrophenyl phosphate (PNPP), Am-plex® Red (AR), naphthol ASMX phosphate, luminol, Lumigen® TMA3, Lumigen® TMA6, sphingosine, 4MUP, L-(+)-2-amino-6-phosphonohexanoic acid, 5-bromo-4-chloro-3-indolyl phosphate (BCIP), BluePhos®, phenylbenzene ω phosphono-α-amino acid, O-phospho-DL-threonine, adenosine monophosphate (AMP), AR (3-amino-9-ethylcarbazole), 4-CN (4-chloro-1-naphtol), DAB (3,3′-DiAminoBenzimidine), OPD (o-phenylene diamine), TMB (3,3″,5,5-tetramethylbenzidine), pNPP (p-nitrophenyl phosphate), NBT (nitroblue tetrazolium), INT (p-iodonitrotetrazolium), MUP (4-methylumbelliferyl phosphate), and FDP fluorescein diphosphate), pyrogallol.

In some embodiments, the reporter enzyme detection probe substrate is selected from:
a. AR, luminol, Lumigen® TMA3, and Lumigen® TMA6, when the reporter enzyme detection probe comprises HRP; or from
b. naphthol ASMX phosphate, and PNPP, when the reporter enzyme detection probe comprises AP.

In some embodiments, the method of detecting a target nucleic acid molecule of the present disclosure further comprises washing the solid phase with the second binding solution prior to incubating the target:detection complex with the reporter enzyme detection probe.

In some embodiments, the method of detecting a target nucleic acid molecule of the present disclosure further comprises washing the solid phase with a blocking agent, optionally bovine serum albumin (BSA), prior to binding the target nucleic acid molecule to the solid phase.

In some embodiments, the substrate reaction solution comprises a non-ionic non polymeric detergent, optionally selected from N-octylglucoside, deoxycholate, rapigest, octyl-beta-glucopyranoside, octylglucopyranoside, chaps, big chap, non-ionic acid labile surfactants, glucosides, n-Octyl-β-D-glucopyranoside, n-Nonyl-β-D-glucopyranoside thioglucosides, n-Octyl-β-D-thioglucopyranoside malto-sides, n-Decyl-β-D-maltopyranoside, n-Dodecyl-β-D-maltopyranoside, n-Undecyl-β-D-maltopyranoside, n-Tridecyl-pi-D-maltopyranoside, cymal-5. cymal-6, thiomaltosides, n-Dodecyl-β-D-thiomaltopyranoside, alkyl glycosides, octyl glucose neopentyl gly-col, polyoxyethylene glycols, triton, NP40, Tween™, Tween™ 20, Triton X-100, triton x-45, C8E4, C8E5, C10E5, C12E8, C12E9, Brij, Anapoe-58, Brij-58, and combinations thereof.

In some embodiments, the substrate reaction solution further comprises 4-iodophenylboronic acid when the substrate comprises luminol.

In some embodiments, the solid phase is a reaction vessel optionally a bead, a plate, a capillary, a filter, or a nano/micro/milli well reaction vessel, and wherein the surface is selected from paper, nitrocellulose, acrylate, plastic, polystyrene, polyvinylene fluoride (PVDF), melamine, silica, polylysine coated glass, 3-aminopropyl-triethoxysilane (APTES) treated glass, and 3-aminopropyl-trimethoxysilane (APTMS) treated glass.

In some embodiments, the attaching of the capture oligonucleotide probe to the solid phase is through H-hydroxysuccinimide (NHS), N-oxysuccinimide (NOS), maleimide, hydrazide, glutaraldehyde coupling, or PEG cross-linking.

In some embodiments, the product ion is assayed by SIM and/or SRM using an optimized fragmentation energy and m/z range.

In some embodiments, the substrate is AMP, ADP or ATP and one or the ionizable products generated comprises adenosine, the product ion of which is assayed by SIM at 268 m/z; or the substrate is CMP, CDP or CTP and one or the ionizable products generated comprises cytosine, the product ion of which is assayed by SIM at 283 m/z; or the substrate is AR and one of the one or more ionizable products generated comprises resorufin, the product ion of which is assayed by SIM at 214 m/z and SRM using the major intense fragment at 214-186 m/z.

In some embodiments, the substrate is naphthol ASMX phosphate and one of the one or more ionizable products generated comprises dephosphorylated naphthol ASMX, the product ion of which is assayed by SIM at 292 m/z and SRM using the major intense fragment at 292-171 m/z or the substrate is PA5P and one or the ionizable products generated comprises PA, the product ion of which is assayed by SIM at 169 m/z.

In some embodiments, the ionizable products are ionized to product ions in ionization solution.

In another aspect, the present disclosure includes a method of quantifying the amount of a target nucleic acid molecule in a test sample comprising the steps:
a. detecting the target nucleic acid molecule according to a method of detecting a target nucleic acid molecule of the present disclosure; and
b. quantifying the amount of target nucleic acid molecule in the test sample based on the intensity of the signal for one or more of the ionizable products detected by mass spectrometry.

In some embodiments, the quantification comprises comparing the intensity of the signal for one or more products against signal intensities generated using known quantities of the target nucleic acid molecule, under similar conditions.

In some embodiments, the target nucleic acid molecule is present or suspected to be present in the sample in or up to a pico mol, femto mol, or atto mol range.

In some embodiments, one or more target oligonucleotide templates are detected.

In some embodiments, the target nucleic acid molecule is a plasmid DNA or a sequence comprised in a bacterial, viral, fungal, mammalian or plant genome.

In some embodiments, the bacterial genome is selected from E. coli, Staphylococcus aureus, Chlamydia, Vibrio cholera, Clostridium, Enterococci, Fusobacterium, anaerobic bacilli, Gram negative cocci, Gram positive bacilli, Haemophilus, Haemophilus influenza, Klebsiella, Lactobacillus, Listeria, Borrelia, Mycobacterium, Mycoplasma, Neisseria, Prevotella, Pseudomonas, Salmonella, Shigella, Spirochaetes, Staphylococcus, Streptococcus, and Yersinia genome.

In some embodiments, the bacterial genome is selected from E. coli, and Staphylococcus aureus.

In some embodiments, the viral genome is selected from HIV, SARS-CoV, MERS, SARS-CoV-2, Ebola virus, influenza virus, coronavirus genome, Enteroviruses, Hepatitis virus, Herpes virus, HPV, Noroviruses, Parainfluenza, Rhinoviruses, and Varicella Virus genome

In some embodiments, the viral genome is selected from HIV, SARS-CoV, MERS, SARS-CoV-2, Ebola virus, influenza virus, and coronavirus genome.

In some embodiments, the fungal genome is selected from Candida genome.

In some embodiments, the mammalian genome is a human genome.

In some embodiments, the target nucleic acid molecule has a sequence comprised in the HIV genome. In some embodiments, the target nucleic acid molecule has a sequenced comprised in the SARS-CoV-2 genome.

In another aspect, the present disclosure includes a method of detecting HIV comprising a method of detecting a target nucleic acid molecule of the present disclosure, wherein the target nucleic acid molecule is a HIV nucleic acid molecule.

In some embodiments, the method of detecting HIV comprises a method of detecting a target nucleic acid molecule of the present disclosure, wherein the capture oligonucleotide probe has a sequence selected from SEQ ID No. 14, SEQ ID No 17, SEQ ID No 20, and SEQ ID No 23.

In some embodiments, the method of detecting HIV comprises a method of detecting a target nucleic acid molecule of the present disclosure, wherein the detection oligonucleotide probe oligonucleotide has a sequence selected from SEQ ID No. 16, SEQ ID No 19, SEQ ID No 22, and SEQ ID No. 25.

In some embodiments, the method of detecting HIV comprises a method of detecting a target nucleic acid molecule of the present disclosure, wherein the capture oligonucleotide probe has a sequence selected from SEQ ID No. 14, SEQ ID No 17, SEQ ID No 20, and SEQ ID No 23.

In another aspect, the present disclosure includes a method of detecting SARS-CoV2 comprising a method of detecting a target nucleic acid molecule of the present disclosure, wherein the target nucleic acid molecule is a SARS-CoV2 nucleic acid molecule.

In some embodiments, the method of detecting SARS-CoV2 of the present disclosure comprises a method of detecting a target nucleic acid molecule of the present disclosure, wherein the capture oligonucleotide probe has a sequence selected from SEQ ID No. 6, and SEQ ID No. 13.

In some embodiments, the method of detecting SARS-CoV2 of the present disclosure comprises a method of detecting a target nucleic acid molecule of the present disclosure, wherein the detection oligonucleotide probe oligonucleotide has a sequence selected from SEQ ID No. 5, and SEQ ID No. 12.

In some embodiments of the method of detecting SARS-CoV2 of the present disclosure, the modified primer has a sequence selected from SEQ ID No. 2, SEQ ID No. 3, SEQ ID No. 7, SEQ ID No. 8, SEQ ID No. 9, and SEQ ID No. 10.

In some embodiments of the method of detecting SARS-CoV2 of the present disclosure, the second primer has a sequence selected from SEQ ID No. 2, SEQ ID No. 3, SEQ ID No. 7, SEQ ID No. 8, SEQ ID No. 9, and SEQ ID No. 10.

In some embodiments of the method of detecting SARS-CoV2 of the present disclosure, the modified primer has sequence of SEQ ID No. 2, and the second primer has sequence of SEQ ID No. 3, or SEQ ID No 8.

In some embodiments of the method of detecting SARS-CoV2 of the present disclosure, the modified primer has sequence of SEQ ID No. 3, and the second primer has sequence of SEQ ID No. 2, or SEQ ID No. 7.

In some embodiments of the method of detecting SARS-CoV2 of the present disclosure, the modified primer has sequence of SEQ ID No.7, and the second primer has sequence of SEQ ID No 3, or SEQ ID No. 8.

In some embodiments of the method of detecting SARS-CoV2 of the present disclosure, the modified primer has sequence of SEQ ID No. 8, and the second primer has sequence of SEQ ID No 2, SEQ ID No. 7.

In some embodiments of the method of detecting SARS-CoV2 of the present disclosure, the modified primer has sequence of SEQ ID No. 9, and the second primer has sequence of SEQ ID No.10.

In some embodiments of the method of detecting SARS-CoV2 of the present disclosure, the modified primer has sequence of SEQ ID No. 10, and the second primer has sequence of SEQ ID No.9.

In some embodiments of the method of detecting SARS-CoV2 of the present disclosure, the modified primer has sequence of SEQ ID No. 38, and the second primer has sequence of SEQ ID No.39.

In some embodiments of the method of detecting SARS-CoV2 of the present disclosure, the modified primer has sequence of SEQ ID No. 39, and the second primer has sequence of SEQ ID No.38.

In some embodiments of the method of detecting SARS-CoV2 of the present disclosure, the modified primer has sequence of SEQ ID No. 41, and the second primer has sequence of SEQ ID No.42.

In some embodiments of the method of detecting SARS-CoV2 of the present disclosure, the modified primer has sequence of SEQ ID No. 42, and the second primer has sequence of SEQ ID No.41.

Other primers could also be used.

In another aspect, the present disclosure includes a kit comprising:
i. a capture oligonucleotide probe, the capture oligonucleotide probe optionally bound of a solid phase, optionally through a linker;
ii. a binding solution comprising a volatile buffer and being substantially free of NaCl or comprising a cross-linking agent;
iii. a detection oligonucleotide probe, the detection oligonucleotide probe comprising an oligonucleotide and a secondary target moiety;
iv. a reporter enzyme detection probe, the reporter enzyme detection probe comprising a reporter enzyme and a secondary target binding moiety capable of binding the secondary target moiety; and/or
v. one or more of: a substrate, a solid phase, a standard, optionally a product ion standard, optionally for preparing a standard curve or tuning calibrant, a second binding solution, a third binding solution, a substrate reaction solution, ionization solution, quenching solution, optionally a second binding solution, detection probe solution, substrate reaction solution, quenching solution, ionization solution as defined herein.

In another aspect, the present aspect includes a kit comprising:
i. a modified primer, the modified primer being functionalized with a secondary target moiety or a reporter enzyme;
ii. a second primer;
iii. when the modified primer is functionalized with the secondary target moiety, a reporter enzyme detection probe, the reporter enzyme detection probe comprising a reporter enzyme and a secondary target binding moiety capable of binding the secondary target moiety; and
iv. one or more of: a substrate, a solid phase, a standard, optionally a product ion standard, optionally for preparing a standard curve or tuning calibrant, a binding solution, a second binding solution, a substrate reaction solution, ionization solution, quenching solution, a washing solution, a cross-linking agent, optionally a binding solution, second binding solution, detection probe solution, substrate reaction solution, quenching solution, ionization solution as defined herein,
   wherein when the modified primer is a forward primer, the second primer is a reverse primer, and when the modified primer is a reverse primer, the second primer is a forward primer.

In some embodiments, the ionization solution comprises an acid or a base, optionally selected from formic acid, acetic acid, trifluoroacetic acid. ammonium hydroxide, methylamine, ethylamine, or propylamine.

In some embodiments, the quenching solution comprises optionally 50% Acetonitrile, 0.1% Acetic acid or 0.1% formic acid or 0.1% trifluoroacetic acid for positive ionization or 0.1% ammonium hydroxide for negative ionization.

In some embodiments, the capture oligonucleotide probe comprises a sequence selected from SEQ ID No. 6, SEQ ID No. 13, SEQ ID No. 14, SEQ ID No 17, SEQ ID No 20, SEQ ID No 23, SEQ ID No 26, SEQ ID No 29, SEQ ID No 32, and SEQ ID No 35.

In some embodiments, the oligonucleotide of the detection oligonucleotide probe comprises a sequence selected from SEQ ID No. 5, SEQ ID No. 12, SEQ ID No. 16, SEQ ID No 19, SEQ ID No 22, SEQ ID No 25, SEQ ID No 28, SEQ ID No 31, SEQ ID No 34, and SEQ ID No 37.

In some embodiments, the capture oligonucleotide probe comprises a sequence of SEQ ID No 14, and the oligonucleotide of the detection oligonucleotide probe has a sequence of SEQ ID No. 16.

In some embodiments, the capture oligonucleotide probe comprises a sequence of SEQ ID No. 6, and the oligonucleotide of the detection oligonucleotide probe has a sequence of SEQ ID No. 5.

In some embodiments, the capture oligonucleotide probe comprises a sequence of SEQ ID No. 13, and the oligonucleotide of the detection oligonucleotide probe has a sequence of SEQ ID No.12.

In some embodiments, the capture oligonucleotide probe comprises a sequence of SEQ ID No 17, and the oligonucleotide of the detection oligonucleotide probe has a sequence of SEQ ID No. 19.

In some embodiments, the capture oligonucleotide probe comprises a sequence of SEQ ID No 20, and the oligonucleotide of the detection oligonucleotide probe has a sequence of SEQ ID No. 22.

In some embodiments, the capture oligonucleotide probe comprises a sequence of SEQ ID No 23, and the oligonucleotide of the detection oligonucleotide probe has a sequence of SEQ ID No. 25.

In some embodiments, the capture oligonucleotide probe comprises a sequence of SEQ ID No 26, and the oligonucleotide of the detection oligonucleotide probe has a sequence of SEQ ID No. 28.

In some embodiments, the capture oligonucleotide probe comprises a sequence of SEQ ID No 29, and the oligonucleotide of the detection oligonucleotide probe has a sequence of SEQ ID No. 31.

In some embodiments, the capture oligonucleotide probe comprises a sequence of SEQ ID No 32, and the oligonucleotide of the detection oligonucleotide probe has a sequence of SEQ ID No. 34.

In some embodiments, the capture oligonucleotide probe comprises a sequence of SEQ ID No 35, and the oligonucleotide of the detection oligonucleotide probe has a sequence of SEQ ID No. 37.

In some embodiments, the capture probe is SEQ ID NO: 44 or 45.

The capture oligonucleotide probe can also be a fragment of a capture probe described herein, for example comprising at least 70%, 80% or 90% of the probe sequence.

In some embodiments, the modified primer and the second primer are primers for a target nucleic acid molecule that has a sequence comprised in a bacterial, viral, fungal, mammalian or plant genome.

In some embodiments, the modified primer has a sequence selected from SEQ ID No. 2, SEQ ID No. 3, SEQ ID No. 7, SEQ ID No. 8, SEQ ID No. 9, SEQ ID No. 10, SEQ ID NO: 38, SEQ ID NO: 39, SEQ ID NO: 41 and SEQ ID NO: 42.

In some embodiments, the second primer has a sequence selected from SEQ ID No. 2, SEQ ID No. 3, SEQ ID No. 7, SEQ ID No. 8, SEQ ID No. 9, SEQ ID No. 10, SEQ ID NO: 38, SEQ ID NO: 39, SEQ ID NO: 41 and SEQ ID NO: 42.

In some embodiments, the modified primer has sequence of SEQ ID No. 2, and the second primer has sequence of SEQ ID No. 3, or SEQ ID No 8.

In some embodiments, the modified primer has sequence of SEQ ID No. 3, and the second primer has sequence of SEQ ID No. 2, or SEQ ID 7.

In some embodiments, the capture oligonucleotide has sequence of SEQ ID No. 6.

In some embodiments, the modified primer has sequence of SEQ ID No.7, and the second primer has sequence of SEQ ID No. 8.

In some embodiments, the modified primer has sequence of SEQ ID No. 8, and the second primer has sequence of SEQ ID No.7.

In some embodiments, the modified primer has sequence of SEQ ID No. 9, and the second primer has sequence of SEQ ID No.10.

In some embodiments, the modified primer has sequence of SEQ ID No. 10, and the second primer has sequence of SEQ ID No.9.

In some embodiments, the capture oligonucleotide has sequence of SEQ ID No. 13.

In some embodiments, the modified primer has sequence of SEQ ID No. 38, and the second primer has sequence of SEQ ID No. 39.

In some embodiments, the modified primer has sequence of SEQ ID No. 39, and the second primer has sequence of SEQ ID No. 38.

In some embodiments, the modified primer has sequence of SEQ ID No.41, and the second primer has sequence of SEQ ID No. 42.

In some embodiments, the modified primer has sequence of SEQ ID No. 42, and the second primer has sequence of SEQ ID No.41.

The primer can also be a fragment of a primer provided herein or comprise additional complementary sequence. For example, the fragment can be at least 70%, 80%, or 90% of the sequence of a primer described herein.

In some embodiments, the capture oligonucleotide has sequence of SEQ ID No. 44 or 45.

In another aspect, the present disclosure includes a nucleic acid of sequence selected from SEQ ID No. 2 to 46.

Also provided is a vector, kit or composition comprising one or more of the nucleic acids of sequence selected from SEQ ID No. 2 to 46.

The nucleic acids can in some embodiments be labelled with a tag. They may also be provided unlabelled optionally in combinations such as in a kit, with a label and reagents for producing the labelled nucleic acid.

### EXAMPLES

The following non-limiting examples are illustrative of the present disclosure.

### General Methods

Alkaline phosphatase streptavidin conjugate (APSA) with a nominal mass of 195,000 kDa (1 mg in 1 mL of 0.01M Tris-HCl, 0.25M NaCl, pH 8.0 with 15 mg/mL Bovine Serum Albumin) was from Jackson Immuno Research Laboratories (West Grove, PA, USA). The AMP substrate and Tris buffer were from Sigma Aldrich (St Louis MO, USA). The NHS-PEG12-Biotin was from Pierce (Thermo Fisher Scientific). The NHS-PEG-NHS was 0,0′-Bis[2-(N-Succinimidyl-succinylamino)ethyl]polyethylene glycol, 2000, from Sigma Aldrich. The 96 well reactive plates were Nunc™ Immobilizer Amino plates from Thermo Fisher Scientific and Corning® DNA-BIND® 96 well plates from Sigma Aldrich. The round cover glass (5 mm Diameter, 0.16-0.19 mm thickness) was from Electron Microscopy Sciences. 3-Aminopropyltriethoxysilane (APTES) is from Thermo Fisher Scientific.

The PVDF membrane can be any common PVDF transfer membrane used for example for Western blots. For example, suitable PVDF membranes include Immobilon-P™ transfer membrane. For example, suitable PVDF membranes can have a pore size of 0.45 μm. For example, PVDF membrane can be in the form of a filter plate, optionally a multiwell filter plate. For example, the bottom of each well of a plate can be fitted with a PVDF membrane. For example, the multiwell plate can be a 96-well filter plate.

The polystyrene support used below is a 1000 Å, C-18 linker attached, non-cleavable spacer polystyrene support obtained from ChemGenes Coporation (Catalog #N-4545-10b). The polystyrene support has the following structure where DMTr refers to dimethyltrityl:

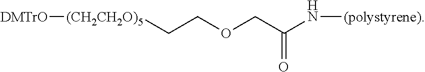

Long-chain alkylamine carboxyl controlled pore glass (CPG long-chain alkylamine support, 500 A pore size, 125-177 micron diameter) may be obtained from Pierce Chem. Co. Sephacryl S-500 may be obtained from Pharmacia. 12% cross-linked polystyrenedivinylbenzene (12% polystyrene-divinylbenzene) resin (200-400 mesh) was purchased from Polysciences.

The nucleocapsid plasmid was obtained from IDT 2019-nCoV_N_Positive Control plasmid (Cat #10006625) and transformed into DH5α from Invitrogen and plated on ampicillin plates, streaked, cultured overnight and then grown for a Qiagen maxi-preps and then quantified by 260/280 ratio. The PCR reactions will created using the ROCHE PCR buffers and with a log titration from 1, 10, 100 zeptomol, 1, 10, 100 attomol, 1, 10, 100 femtomol, 1, 10 picomol of the nucleocapsid plasmid obtained from plasmid per reaction in a Bio-Rad T100 Thermo Cycler for 35 cycles. The PCR products less than 300 bases were resolved by TBE PAGE for quantification by Gelred alongside standard and cut plasmid quantitative standard curve run into the gel.

The model 1100 HPLC was from Agilent (Santa Clara, CA, USA). The model 7725 injector was from Rheodyne (IDEX, Rohnert Park, CA). The LTQ XL linear quadrupole ion trap was from Thermo Electron Corporation (Waltham, MA, USA). The Zorbax 3.5 micron 300 Å C18 resin was from Chromatographic Specialties (Brockville, ON, CANADA).

The APSA enzyme that is a universal biotin binding signal amplification enzyme conjugate showed a linear range from 1 pg to 50 pg per 96 well with BCIP/NBT in pH 8.85 20 mM Tris by UV/VIS detection at around 600 nm.

APSA was dissolved in Reaction Buffer (20 mM Tris, pH 8.85) for assay by colorimetric reaction with BCIP/NBT dye substrate to form indigo blue in 0.1% Tween 20, and measured at 595 nm on a 96 well plate reader (Bio-Rad). Adenosine served as an absolute standard for LC-ESI-MS reactions and was dissolved in 70% acetonitrile (ACN) with 0.1% acetic acid. In parallel, APSA was reacted with AMP to form adenosine that may be sensitively detected by LC-ESI-MS. For “DNA ELiMSA” assays, the APSA was dissolved in 10 ml of reaction buffer of 20 mM Tris, pH 8.85, to yield a 1 ng per μL stock. The APSA 1 ng/μL was diluted in series by dissolving 10 μL in 10 ml reaction buffer to yield 1 pg/μL and then the working stock of 1 fg/μL (1000 ag/μL). The 1 fg/μL working stock was used to make a linear dilution series from 0.1 to 1000 femtogram per ml of buffer and reacted at 37° C. with 1 μM to 1 mM AMP for 2 h. For LC-ESI-MS/MS assays the reaction was quenched 1:1 (DF 2) in acetonitrile with 0.2% acetic acid on ice and then loaded into a 96 well plate autosampler injecting 2 μL with isocratic separation at 200 μL per minute with an Agilent 1100 HPLC over 5 micron C18 (2.1 mm×150 mm) in 7.5% or 95% acetonitrile with 0.1% acetic acid at 20 μL/min for LC-ESI-MS with a linear quadrupole ion trap (Thermo) tuned with adenosine at 268.2 [M+H]⁺. The AMP substrate and adenosine product from the enzyme conjugate APSA were quantified in the SIM mode and the adenosine peak data extracted after subtracting and averaging local background adjacent to the 268.24 [M+H]⁺ m/z chromatographic peak at about 1.2 minutes. Alternatively the SRM product of MS/MS: Full scan: m/z 120 to 400 m/z SRM: 268→136, isolation window: 2 Da, Collision energy 35 CID was monitored.

Blocking buffer can be but is not limited to a serum-based, BSA or Albumin based, polylysine-based, fibronectin-based, gelatin-based, or skim milk powder-based buffer. The blocking buffer can further comprise detergents such as non-ionic detergents including deoxycholate, n-octylglucoside N-octyl-β-glucopyranoside, Big CHAP deoxy, acid-cleavable detergent, EDTA. The blocking buffer can further comprise a buffering agent such as TRIS. It may be appreciated by a person skilled in the art that other blocking buffers similar to the ones described above can also be used depending on the specific application of the methods of the present disclosure.

Binding buffer can be but is not limited to TRIS, PBS, HEPES, MES or MOPS-based buffer. It may be appreciated by a person skilled in the art that other binding buffers similar to the ones described above can also be used depending on the specific application of the methods of the present disclosure. In some instances, the binding buffer can further comprise other components such as salts. In the case where the binding buffer comprises salts, for the MS analysis, the sample containing the one or more ionizable products may be optionally run with a salt divert valve to prevent salt from reaching the ionization source. Alternatively or additionally, the sample containing the one or more ionizable products may also be desalted by chromatography (for example using C18 chromatography column) prior to the MS analysis. Further, the sample containing the one or more ionizable products may also be diluted in organic solvent and centrifuged prior to injection.

### EXAMPLE 1 DETECTION OF NUCLEIC ACID ON HIGH BINDING 0.45 MICRON PVDF 96-WELL FILTER PLATES

The following shows a general method of nucleic acid adsorption and detection on a PVDF filter plate.
Capture oligonucleotide probe adsorption to the PVDF filter plate: Pre-wet the PVDF with methanol; spot 10 μL 100 μM capture oligonucleotide probe per well; let it dry for 1 h under fume hood.
Blocking: Add 200 μL Blocking Solution (3% (w/v) BSA in 20 mM Tris pH8.00+1 mM EDTA) per well, incubate for 1 h at 37° C., and wash 3×2 min with 20 mM Tris pH8.00+1 mM EDTA (same for the following washing steps).
Washing: Wash the wells 3× with Binding Buffer (20 mM Tris pH8.00+1 M NaCl+1 mM EDTA).
Target nucleic acid molecule hybridization:
   Dilute target nucleic acid molecule (1 μM) and detection oligonucleotide probe 1/10 in Binding Buffer. Add 100 μL Binding Buffer+detection oligonucleotide probe 1/10 10 μL for 0 target nucleic acid molecule vs. 100 uL 1 μM target nucleic acid molecule+detection oligonucleotide probe 1/10 10 μL for 1 μM target nucleic acid molecule (100 fmol Target injected) per reaction, and incubate at 90° C. for 15 min.
   Pre-incubate the plate to the hybridization temperature, 60° C. Add 110 μL of the target nucleic acid molecule and detection oligonucleotide probe mixture solution per well, and incubate for 1 h.
washing (optional): Wash 3× with 200 μL Binding Buffer.
Reporter enzyme detection probe binding: Dissolve 10 μg (10 μL) reporter enzyme detection probe (e.g. APSA) in 1 mL Binding Buffer, and further dilute reporter enzyme detection probe 1/100 in Binding Buffer (10 ng/ml); add reporter enzyme detection probe dilution 100 μL per well (1 ng), and incubate at 37° C. for 15 min.
Washing: Wash 9× with 20 mM Tris pH8.00+1 M NaCl (no EDTA), and remove the last bit of solution in the well.
Reporter enzyme reaction: Add 100 μL of 1 mM reporter enzyme detection probe substrate (e.g. AMP) in substrate reaction solution (20 mM Tris pH 8.85) and incubate 2 h at 37° C.
Collect 50 μL per well of the reactant and transfer it to a new tube.
Quench the reaction 1:1 with 50 μL 0.2% acetic acid in 100% acetonitrile (ACN) (HPLC), and then diluted 1:10 (final DF20) for MS analysis in Scan mode.

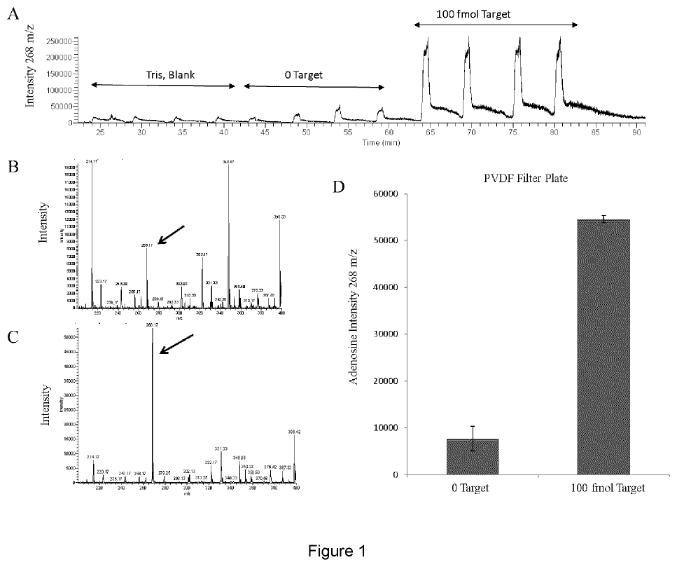

Using the target nucleic acid molecule, the capture oligonucleotide and the detection oligonucleotide sequences of HIV viral DNA listed in Table 6 as examples, immobilization of the specific Capture oligonucleotide probe by adsorption to Immobilon-P™ PVDF membrane in 96 well plates resulted in a signal intensity of over 55,000 arbitrary counts on a background of less than 7,000 counts from the specific detection DNA. The signal for 100 fmol of target viral DNA on column 4 independent replicates is shown in FIG. 1.

### Example 2 Detection of Nucleic Acid Crosslinked to Polylysine Coating in a 96 Well Polystyrene Plate

The following shows a general method of nucleic acid adsorption and detection on a polylysine coated polystyrene plate.
Polylysine coating the polystyrene plate: Add 0.01% poly-L-lysine solution (Sigma P4707) 100 μL per well and incubate overnight (16-18 h) at 4° C.; washed 3×2 min with 200 μL 1×PBS, pH7.2, on a tilting shaker (Same to the following washing steps)
Capture oligonucleotide probe crosslinking: Add 1 mM NHS-PEG-NHS 1×PBS solution to the dissolved 1 μM aminated Capture oligonucleotide probe solution in 1×PBS at 10 μM final concentration (DF100, 10 μL per 1 ml Capture oligonucleotide probe solution); transfer 100 μL to each well and incubate 30 min at 37° C.; wash 3× with 1×PBS.
Quenching and blocking: Add 200 μL 3% (w/v) BSA in 20 mM Tris pH8.00+1 mM EDTA per well, incubate for 1 h at 37° C., and wash 3× with 20 mM Tris pH8.00+1 mM EDTA.
Washing (optional): Wash the wells 3× with Binding Buffer (20 mM Tris pH8.00+1 M NaCl+1 mM EDTA)
Target nucleic acid molecule hybridization: (Same as Example 1)
Washing: (Same as Example 1)
Reporter enzyme detection probe binding: (Same as Example 1)
Washing (optional): (Same as Example 1)
Reporter enzyme reaction: (Same as Example 1)

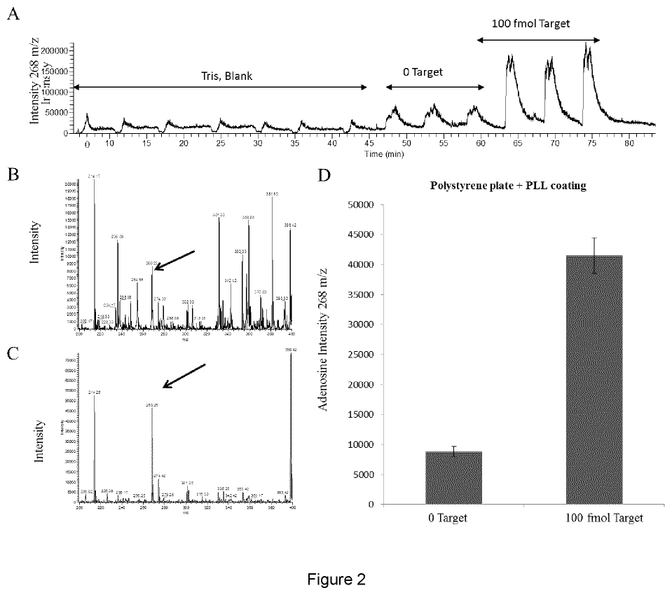

Using the target nucleic acid molecule, the capture oligonucleotide and the detection oligonucleotide sequences of HIV viral DNA listed in Table 6 as examples, a signal intensity of 42,000 counts on a background of about 8,000 counts was observed when viral target nucleic acid molecule was captured on polystyrene plates coated with polylysine and crosslinked with an 5′- or 3′-aminated viral Capture oligonucleotide probe by NHS-PEG-NHS and the equivalent of 100 fmol of captured target nucleic acid molecule was injected on column. The results from the equivalent of 100 fmol target nucleic acid molecule injected on column from 3 independent replicates are shown FIG. 2.

### Example 3 Detection of Nucleic Acid on Amine-Reactive Nunc Immobilizer Amino 96 Well Polystyrene Plate

The following shows a general method of nucleic acid adsorption and detection on an amine-reactive Nunc Immobilizer™ Amino polystyrene plate.
Capture oligonucleotide probe immobilization to the plate: Dilute Capture oligonucleotide probe Stock (100 μM) 1/10 in Surface Binding Buffer (100 mM Sodium Carbonate buffer, pH 9.6), and add 100 μL of the dilution per well; incubate overnight at 4° C.; wash 3×2 min with 200 μL Surface Binding Buffer on a tilt table (Same to the following washing steps)
Quenching and blocking: Add 200 μL 3% (w/v) BSA in 20 mM Tris pH8.00+1 mM EDTA per well, incubate for 1 h at 37° C., and wash 3× with 20 mM Tris pH8.00+1 mM EDTA
Washing: Wash 3× with Binding buffer (20 mM Tris pH8.00+1 M NaCl+1 mM EDTA)
Target nucleic acid molecule hybridization: (Same as Example 1)
Washing (optional): (Same as Example 1)
Reporter enzyme detection probe binding: (Same as Example 1)
Washing: (Same as Example 1)
Reporter enzyme reaction: (Same as Example 1)

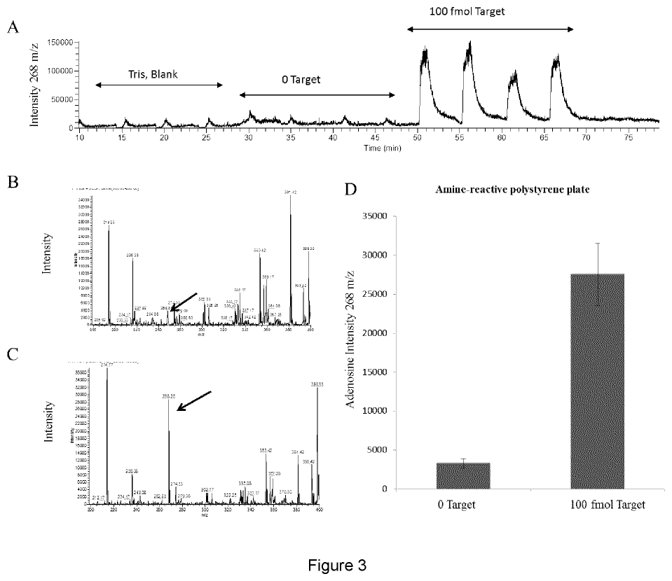

Using the target nucleic acid molecule, the capture oligonucleotide and the detection oligonucleotide sequences of HIV viral DNA listed in Table 6 as examples, immobilization of the 5′- or 3′-aminated viral Capture oligonucleotide probe via amine-reactive functional groups (reactive carboxylic functional groups) in 96 well Nunc Immobilizer Amino plates resulted in 27,000 counts on a background of 3,000 counts. The results from the equivalent of 100 fmol Target nucleic acid molecule injected on column are shown in FIG. 3.

### Example 4 Detection of Nucleic Acid on NOS Surface Chemistry 96 Well Polystyrene Reactive Plate

The following shows a general method of nucleic acid adsorption and detection on an N-oxysuccinimide (NOS) surface chemistry polystyrene plate.
Capture oligonucleotide probe immobilization to the plate: Dilute Capture DNA Stock (100 uM) 1/10 in Surface Binding Buffer (10 mM Na₂PO₄+1 mM EDTA buffer, pH 8.5), and add 100 μL per well, incubate overnight at 4° C., decant the solution and wash 3×2 min with Surface Binding Buffer
Quenching and blocking: Block and quench the surface with 200 μL 3% (w/v) BSA in 20 mM Tris pH8.00+1 mM EDTA per well, incubate for 1 h at 37° C., decant, and wash 3× with 20 mM Tris pH8.00+1 mM EDTA.
Washing: Wash 3× with Binding Buffer (20 mM Tris pH8.00+1 M NaCl+1 mM EDTA).
Target nucleic acid molecule hybridization: (Same as Example 1)
Washing: (Same as Example 1)
Reporter enzyme detection probe binding: (Same as Example 1)
Washing (optional): (Same as Example 1)
Reporter enzyme reaction: (Same as Example 1)

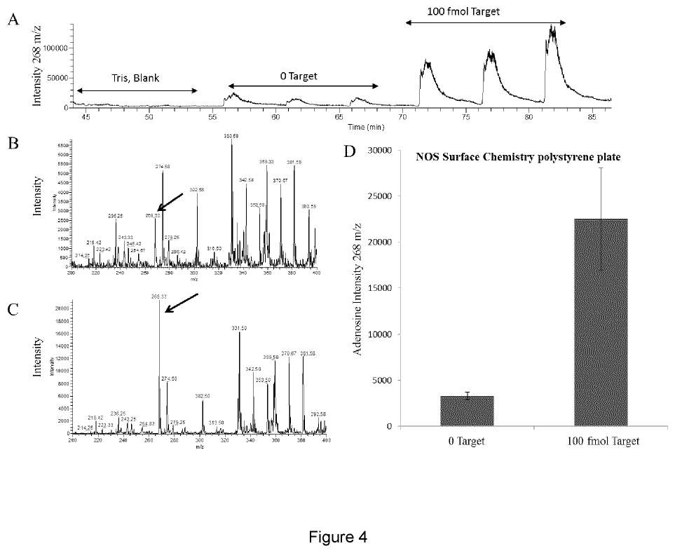

Using the target nucleic acid molecule, the capture oligonucleotide and the detection oligonucleotide sequences of HIV viral DNA listed in Table 6 as examples, immobilization of 5′- or 3′-aminated viral Capture oligonucleotide probe by N-oxysuccinimide (NOS) surface chemistry in Corning® DNA-BIND® 96 well plates for capturing target nucleic acid molecule resulted in 22,000 specific counts compared to a background of about 3,000 counts. The results from the equivalent of 100 fmol Target DNA injected on column from 3 independent replicates are shown in FIG. 4.

### Example 5 Detection of Nucleic Acid 3′ Linked to Polystyrene Oligosynthesis Beads in a 96 Well PVDF Filter Plate

The following shows a general method of nucleic acid detection where the capture oligonucleotide probe is 3′ linked polystyrene oligosynthesis beads in a PVDF filter plate.
Blocking the filterplate: Add 200 μL Blocking solution (3% (w/v) BSA in 20 mM Tris pH8.00+1 mM EDTA) per well, incubate for 1 h at 37° C., decant, and wash 3× with 20 mM Tris pH8.00+1 mM EDTA using the vacuum manifold setup (Same to the following washing steps).
Washing: Wash 3× with Binding buffer (20 mM Tris pH8.00+1 M NaCl+1 mM EDTA)
Target nucleic acid hybridization:
   Use 10 μL of Capture oligonucleotide probe bead suspension per well. Pellet beads (5 minutes at 16,000 RCF) in the centrifuge tube, and remove supernatant.
   Re-suspend Capture beads in 500 μL Blocking Solution for 15 minutes at RT on Ferris Wheel.
   Wash beads 2× in 1 mL 20 mM Tris pH 8.00+1 mM EDTA and centrifuge for 5 min at 16,000 RCF to remove supernatant.
   Re-suspend Capture beads in Binding Buffer (20 mM Tris pH8.00+1 M NaCl+1 mM EDTA).
   Dilute Target nucleic acid molecule (1 μM) and capture oligonucleotide probe 1/10 in Binding Buffer, and add 100 μL Binding Buffer+capture oligonucleotide probe 1/10 10 μL for 0 Target nucleic acid molecule vs. 100 μL1 μM Target nucleic acid molecule+capture oligonucleotide probe 1/10 10 μL for 1 μM Target nucleic acid molecule per reaction to each tube with Capture beads.
   Incubate at 90° C. for 15 min and then at 60° C. for 1 h
   Transfer the DNA mixture to the filter plate.
Washing (optional): Wash 3× with 200 μL Binding Buffer per well.
Reporter enzyme detection probe binding: (Same as Example 1)
Washing: (Same as Example 1)
Reporter enzyme reaction: (Same as Example 1)

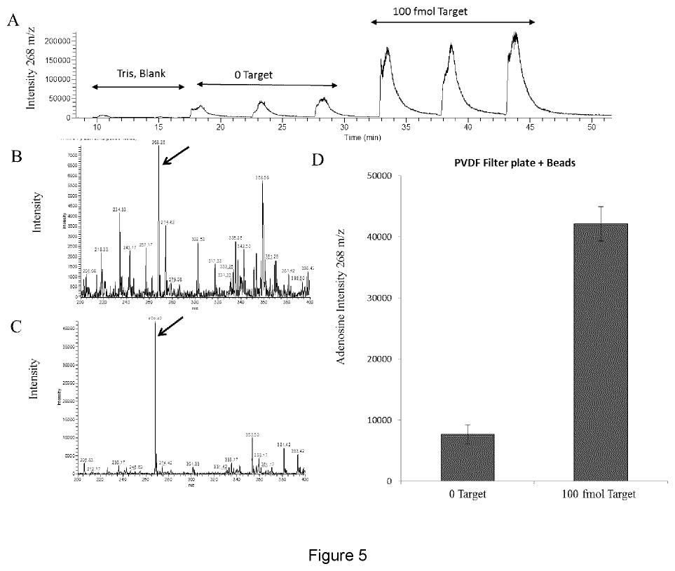

Using the target nucleic acid molecule, the capture oligonucleotide and the detection oligonucleotide sequences of HIV viral DNA listed in Table 6 as examples, viral DNA captured by Capture oligonucleotide probe with 3′ links to polystyrene oligosynthesis beads in 96 well PVDF filter plate resulted in a signal of 42,000 specific counts compared to a background of about 8,000 counts. The results from the equivalent of 100 fmol Target nucleic acid molecule injected on column from 3 independent replicates are shown in FIG. 5.

### Example 6 Detection of Nucleic Acid with Capture Oligonucleotide Probe Crosslinked to Amino-Silylated Cover Glass Surface

The following shows a general method of nucleic acid detection where the capture oligonucleotide probe is crosslinked to amino-silylated cover glass surface.
Amino-silylation of the cover class:
   Thoroughly wash the cover glass by soaking it into 2.5M NaOH overnight, wash with DI water, immerse into 10% HCl, rinse with DI water and methanol, and allow surface to air-dry.
   Prepare a 2% solution of ATPES in acetone, and immerse the cover glass in the solution for 15 min. Rinse the cover glass with acetone and leave it to air-dry.
   Carefully put the cover glass into the well of a 96 well polystyrene plate.
Capture oligonucleotide probe crosslinking: Add 1 mM NHS-PEG-NHS 1×PBS solution to the dissolved 1 μM Capture oligonucleotide probe solution in 1×PBS at 10 μM final concentration (10 uL per 1 ml Capture oligonucleotide probe solution); transfer 100 uL per well and incubate 30 min at 37° C.; wash 3× with 1×PBS.
Quenching and blocking: Add 200 μL 3% (w/v) BSA in 20 mM Tris pH8.00+1 mM EDTA per well, incubate for 1 h at 37° C., and wash 3× with 20 mM Tris pH8.00+1 mM EDTA.
Washing: Wash the wells 3× with Binding Buffer (20 mM Tris pH8.00+1 M NaCl+1 mM EDTA).
Target nucleic acid molecule hybridization: (Same as the previous Example 1)
Washing (optional): (Same as Example 1)
Reporter enzyme detection probe binding: (Same as Example 1)
Washing: (Same as Example 1)
Reporter enzyme reaction: (Same as Example 1)

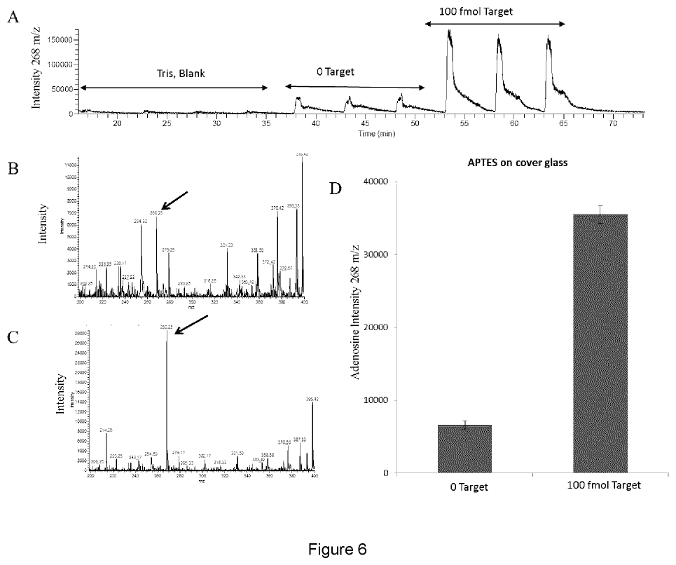

Using the target nucleic acid molecule, the capture oligonucleotide and the detection oligonucleotide sequences of HIV viral DNA listed in Table 6 as examples, immobilization of the 5′- or 3′-aminated viral Capture oligonucleotide probe on a glass surface via the amino-silylation of the glass and crosslinking by NHS-PEG-NHS resulted in 35,000 counts on a background of 6,000 counts. The results from the equivalent of 100 fmol Target nucleic acid molecule injected on a column from 3 independent replicates are shown in FIG. 6.

### Example 7 Optimization of NaCl in Binding Buffer for DNA

### Buffer Optimization

Buffer optimization experiments are described in Examples 7 and 8 using capture oligonucleotide probe linked to polystyrene oligosynthesis beads in 96 well 0.45 μm PVDF filter plates as previously described in Example 5 with slight modifications.

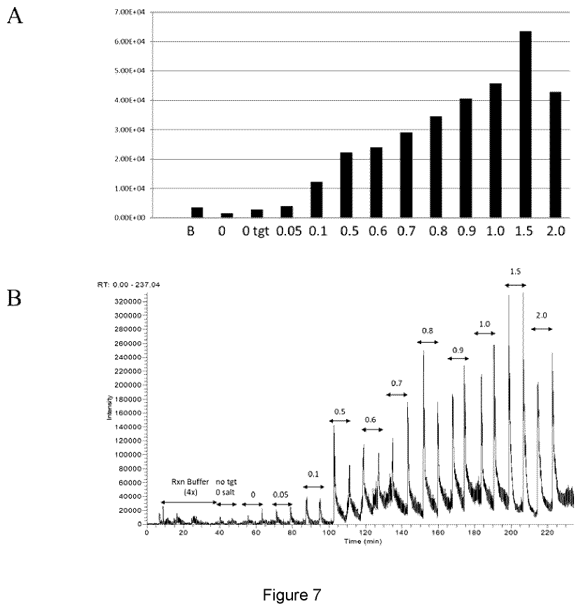

The PVDF filter plate was blocked with 3% BSA for 1 h and washed 3 times with 20 mM Tris-HCl, 1 mM EDTA, pH8.0 and equilibrated in binding buffers of various NaCl concentrations: 0, 0.05, 0.1, 0.5, 0.6, 0.7, 0.8, 0.9, 1.0, 1.5 and 2.0 M. Separately, capture beads were blocked with 3% BSA for 15 min and washed in 20 mM Tris-HCl, 1 mM EDTA by centrifugation and equilibrated in the various binding buffers. The capture oligonucleotide probe and target nucleic acid molecule were applied to the beads in the various binding buffers for DNA hybridization at 90° C. for 15 minutes followed by 60° C. for 1 hour. Beads were transferred to the PVDF filter plates, washed 3 times in the various binding buffers and incubated for 15 min at 37° C. with APSA. Unbound APSA was washed away in 9 washes of the various binding buffers and the beads were incubated with 1 mM AMP substrate for 2 hours in 20 mM Tris-HCl, pH 8.85. The reaction was quenched and diluted 1:20 in 100% and a final concentration of 0.1% acetic acid. The samples were separated by a C₁₈ reverse phase 3.5 um column with a 100% acetonitrile, 0.1% acetic acid mobile phase and the adenosine product was detected by an LTQ linear ion trap at 268.2 m/z [M+H]⁺. Each sample was injected twice and the NaCl optimum was identified at 1.5M. The results of MS signal intensity at different concentrations of NaCl are shown in FIG. 7.

### Example 8 Optimization of Ammonium Bicarbonate in Binding Buffer for DNA

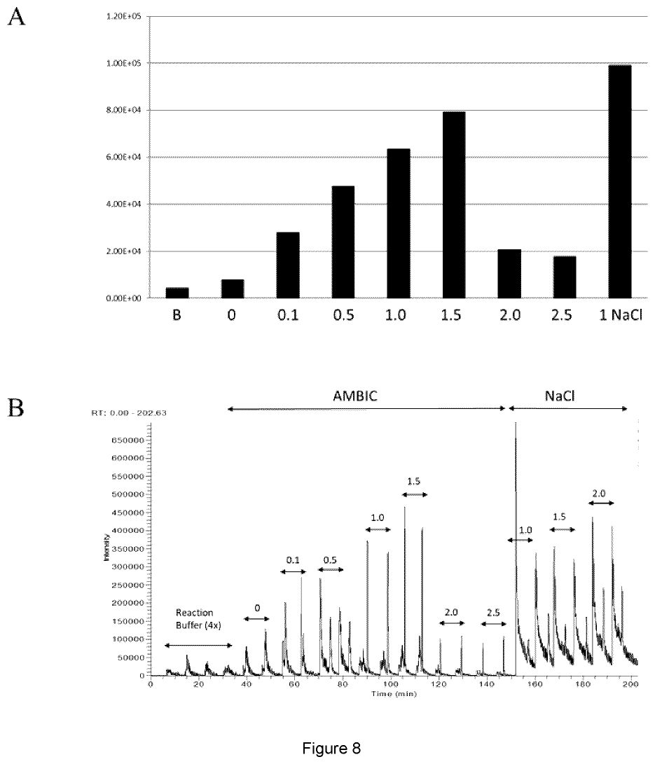

The PVDF filter plate was blocked with 3% BSA for 1 h and washed 3 times with 20 mM Tris-HCl, 1 mM EDTA, pH8.0 and equilibrated in binding buffers of various ammonium bicarbonate concentrations: 0, 0.1, 0.5, 1.0, 1.5, 2.0, 2.5 M. Separately, capture beads were blocked with 3% BSA for 15 min and washed in 20 mM Tris-HCl, 1 mM EDTA by centrifugation and equilibrated in the various binding buffers. The capture oligonucleotide probe and target nucleic acid molecule were applied to the beads in the various binding buffers for DNA hybridization at 90° C. for 15 minutes followed by 60° C. for 1 hour. Beads were transferred to the PVDF filter plates washed 3 times in the various binding buffers and incubated for 15 min at 37° C. with APSA. Unbound APSA was washed away in 9 washes of the various binding buffers and the beads were incubated with 1 mM AMP substrate for 2 hours in 20 mM Tris-HCl, pH 8.85. The reaction was quenched and diluted 1:20 in 100% and a final concentration of 0.1% acetic acid. The samples were separated by a C18 reverse phase 3.5 um column with a 100% acetonitrile, 0.1% acetic acid mobile phase and the adenosine product was detected by an LTQ linear ion trap at 268.2 m/z [M+H]⁺. Each sample was injected twice and the ammonium bicarbonate optimum was identified at 1.5M. The results are shown in FIG. 8.

The target nucleic acid molecule, capture oligonucleotide probe and detection oligonucleotide probe are HIV sequences are shown in Table 6.

### Example 9 Comparison of Volatile Buffers in Binding Buffer after Hybridization

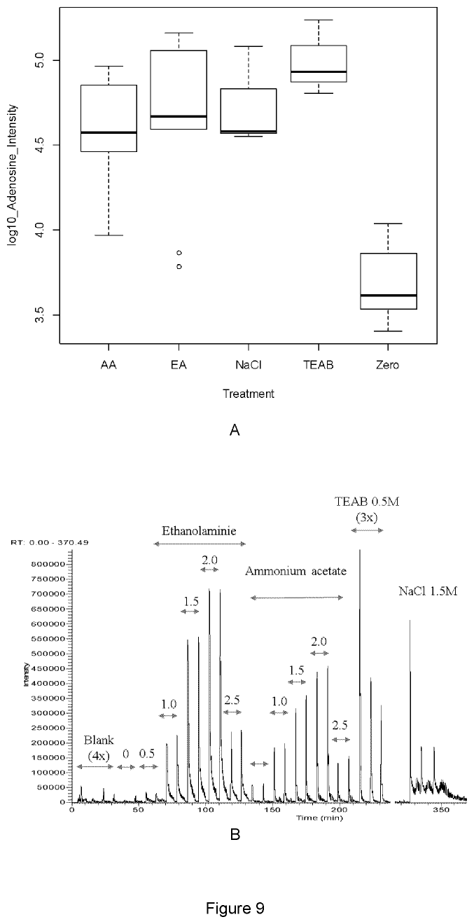

The PVDF filter plate was blocked with 3% BSA for 1 h and washed 3 times with 20 mM Tris-HCl, 1 mM EDTA, pH8.0 and equilibrated in 1.5M NaCl, 20 mM Tris-HCl, 1 mM EDTA, pH8.0 (binding buffer). Separately, capture beads were blocked with 3% BSA for 15 min and washed in 20 mM Tris-HCl, 1 mM EDTA by centrifugation and equilibrated in binding buffer. The capture oligonucleotide probe and target nucleic acid molecule were applied to the beads in binding buffer for DNA hybridization at 90° C. for 15 minutes followed by 60° C. for 1 hour. Beads were transferred to the PVDF filter plates washed 3 times in binding buffers where the 1.5M NaCl was replaced by either: 0.5, 1.0, 1.5, 2.0, 2.5 M ethanolamine, 0.5, 1.0, 1.5, 2.0, 2.5 M ammonium acetate, 0.5M triethyl ammonium bicarbonate, 0.5, 1.0, 1.5, 2.0, 2.5 M ammonium bicarbonate or the standard 1.5M NaCl. APSA was applied the beads in the various binding buffers and incubated for 15 min at 37° C. Unbound APSA was washed away in 9 washes of the various binding buffers and the beads were incubated with 1 mM AMP substrate for 2 hours in 20 mM Tris-HCl, pH 8.85. The reaction was quenched and diluted 1:20 in 100% and a final concentration of 0.1% acetic acid. The samples were separated by a C18 reverse phase 3.5 um column with a 100% acetonitrile, 0.1% acetic acid mobile phase and the adenosine product was detected by an LTQ linear ion trap at 268.2 m/z [M+H]⁺. Each sample was injected twice and the best performing volatile buffer was 2M ethanolamine. The results are shown in FIG. 9.

### Example 10 PCR Primers, Oligonucleotide Capture Probe and Oligonucleotide Detection Probe Sequences

PCR primers and oligo capture and detection DNA sequences were designed using the NCBI PCR and oligo DNA algorithm PCR-BLAST that takes into account the interfering effects of miRNA and ncRNA (Tables 1 to 6). A high false negative rate observed in ruPR reactions of for SARS-CoV-2 (Xie, 2020). A flexible set of PCR primers and/or nested oligo capture sequences were designed to amplify and then capture the SARS-CoV-2 PCR products for a second stage amplification by alkaline phosphatase and LC-ESI-MS detection. The primers are compared to those recommended by the World Health Organization as a control.

Design considerations for oligonucleotide hybridization probes specific for SARS-CoV-2 nucleocapsid gene (SEQ ID No.1) that is homologous to SARS and MERS showing region targeted by the NCBI Primer-BLAST algorithm in the NC_045512.2:28274-29533 Wuhan seafood market pneumonia virus isolate Wuhan-Hu-1, nucleocapsid gene. In Table 1, capture and detection oligo sites are shown in bold underline.

TABLE 1
SARS-COV-2 nucleocapsid sequence
SARS-COV-2 Nucleocapsid

ATGTCTGATAA [symbol] ATGCACCCCGCATTACGTTTGGTGGACCCTCAGATTCAA
CTGGCAGTAACCAGAATGGAGAACGCAGTGGGGCGCGATCAAAACAACGTCGGCCCCAAGGTTTACCCAA
TAATACTGCGTCTTGGTTCACCGCTCTCACTCAACATGGCAAGGAAGACCTTAAATTCCCTCGAGGACAA
GGCGTTCCAATTAACACCAATAGCAGTCCAGATGACCAAATTGGCTACTACCGAAGAGCTACCAGACGAA
TTCGTGGTGGTGACGGTAAAATGAAAGATCTCAGTCCAAGATGGTATTTCTACTACCTAGGAACTGGGCC
AGAAGCTGGACTTCCCTA [symbol] TCATATGGGTTGCAACTGAGGGAGCCTTGAAT
ACACCAAAAGATCACATTGGCACCCGCAATCCTGCTAACAATGCTGCAATCGTGCTACAACTTCCTCAAG
GAACAACATTGCCAAAAGGCTTCTACGCAGAAGGGAGCAGAGGCGGCAGTCAAGCCTCTTCTCGTTCCTC
ATCACGTAGTCGCAACAGTTCAAGAAATTCAACTCCAGGCAGCAGTAGGGGAACTTCTCCTGCTAGAATG
GCTGGCAATGGCGGTGATGCTGCTCTTGCTTTGCTGCTGCTTGACAGATTGAACCAGCTTGAGAGCAAAA
TGTCTGGTAAAGGCCAACAACAACAAGGCCAAACTGTCACTAAGAAATCTGCTGCTGAGGCTTCTAAGAA
GCCTCGGCAAAAACGTACTGCCACTAAAGCATACAATGTAACACAAGCTTTCGGCAGACGTGGTCCAGAA
CAAACCCAAGGAAATTTTGGGGACCAGGAACTAATCAGACAAGGAACTGATTACAAACATTGGCCGCAAA
TTGCACAATTTGCCCCCAGCGCTTCAGCGTTCTTCGGAATGTCGCGCATTGGCATGGAAGTCACACCTTC
GGGAACGTGGTTGACCTACACAGGTGCCATCAAATTGGATGACAAAGATCCAAATTTCAAAGATCAAGTC
ATTTTGCTGAATAAGCATATTGACGCATACAAAACATTCCCACCAACAGAGCCTAAAAAGGACAAAAAGA
AGAAGGCTGATGAAACTCAAGCCTTACCGCAGAGACAGAAGAAACAGCAAACTGTGACTCTTCTTCCTGC
TGCAGATTTGGATGATTTCTCCAAACAATTGCAACAATCCATGAGCAGTGCTGACTCAACTCAGGCCTAA
(SEQ ID No.1)

Capture and detection regions are underlined. Italics indicate primer regions.

A first set (SARS CoV2 Set 1) of PCR primers for SARS-CoV-2 nucleocapsid are shown in Table 2. Abbreviations: FP, forward primer; RP, reverse primer; P/N polystyrene oligosynthesis bead/covalent Amine link 96 well plate; B, biotin. Optionally, the primers (e.g. SEQ ID No. 2) can be functionalized with biotin and/or be conjugated to a polystyrene oligosynthesis bead.

TABLE 2
SARS COV2 Set 1 of PCR Primers for SARS-COV-2

Forward Primers
5′-CAAAACAACGTCGGCCCCAAGG-3′ (SEQ ID No. 2)
B-5′-CAAAACAACGTCGGCCCCAAGG-3′ (SEQ ID No. 2)
P/N-5′-CAAAACAACGTCGGCCCCAAGG-3′ (SEQ ID No. 2)
Reverse Primers
5′-GGTCATCTGGACTGCTATTGGTGT-3′ (SEQ ID No. 3)
B-5′-GGTCATCTGGACTGCTATTGGTGT-3′ (SEQ ID No. 3)
P/N-5′-GGTCATCTGGACTGCTATTGGTGT-3′(SEQ ID No. 3)

A second set (SARS CoV2 Set 2) of PCR primers for SARS-CoV-2 nucleocapsid gene, an example of a corresponding target nucleic acid molecule sequence and an exemplary set of corresponding capture and detection oligonucleotide probes are shown in Table 3. Abbreviations: C, capture oligo; D, detection oligo; FP, forward primer; RP, reverse primer; P/N polystyrene oligosynthesis bead/covalent Amine link 96 well plate; b, biotin; PCR, reaction product.

TABLE 3
SARS COV2 Set 2 PCR Primers and probe Design
for SARS-COV-2

Forward Primers
5′-CAAAACAACGTCGGCCCCAAGG-3′
(SEQ ID No. 2)
Reverse Primers
5′-GGTCATCTGGACTGCTATTGGTGT-3′
(SEQ ID No. 3)
Target nucleic acid molecule for SARS-COV-2
5′-
CAAAACAACGTCGGCCCCAAGGTTTACCCAATAATACTGCGTCTTGGTTC
ACCGCTCTCACTCAACATGGCAAGGAAGACCTTAAATTCCCTCGAGGACA
AGGCGTTCCAATTAACACCAATAGCAGTCCAGATGACC-3′
(SEQ ID No. 4)
Detection probe oligonucleotide
5′-GGTCATCTGGACTGCTATTGGTGTTAATTGGAACGCCTTGTCCTCGA
GGG-3′
(SEQ ID No. 5)
B-5′-GGTCATCTGGACTGCTATTGGTGTTAATTGGAACGCCTTGTCCTC
GAGGG-3′
(SEQ ID No. 5)
Capture oligonucleotide probe sequence
5′-GAACCAAGACGCAGTATTATTGGGTAAACCTTGGGGCCGACGTTGTT
TTG-3′
(SEQ ID No. 6)
5′-GAACCAAGACGCAGTATTATTGGGTAAACCTTGGGGCCGACGTTGTT
TTG-3′-P/N
(SEQ ID No. 6)

A third set (SARS CoV2 Set 3) of PCR primers for the SARS-CoV-2 Nucleocapsid sequence is shown in Table 4. Abbreviations: FP, forward primer; RP, reverse primer; P/N polystyrene oligosynthesis bead/covalent Amine link 96 well plate; B, biotin.

TABLE 4
SARS COV2 Set 3 PCR Primers

Forward Primer
5′-TGGACCCCAAAATCAGCGAA-3′
(SEQ ID No. 7)
b-5′-TGGACCCCAAAATCAGCGAA-3′
(SEQ ID No. 7)
P/N-5′-TGGACCCCAAAATCAGCGAA-3′
(SEQ ID No. 7)
Reverse primer
5′-TGCCGTCTTTGTTAGCACCA-3′
(SEQ ID No. 8)
b-5′-TGCCGTCTTTGTTAGCACCA-3′
(SEQ ID No. 8)
P/N-5′-TGCCGTCTTTGTTAGCACCA-3′
(SEQ ID No. 8)

Another set (SARS CoV2 Set 4) of PCR primer design for SARS-CoV-2, an example of a corresponding target nucleic acid molecule sequence and an exemplary set of corresponding capture and detection oligonucleotide probes are shown in Table 5. Abbreviations: FP, forward primer; RP, reverse primer; P/N polystyrene oligosynthesis bead/amine link; b, biotin. A longer reaction product with the same capture and detection oligonucleotides (Table 3) results from the primers: Forward, 5′-TGGACCCCAAAATCAGCGAA-3′ (SEQ ID No. 7); Reverse, 5′-TGCCGTCTTTGTTAGCACCA-3′ (SEQ ID No. 8).

TABLE 5
SARS COV2 Set 4 PCR Primer and capture/detection
probe design for SARS-COV-2

Forward Primers
5′-CGAGGACAAGGCGTTCCAAT-3′
(SEQ ID No. 9)
Reverse Primers
5′-TGTTAGCAGGATTGCGGGTG-3′
(SEQ ID No. 10)
Target nucleic acid molecule for SARS-COV-2
5′-
GCGTTCCAATTAACACCAATAGCAGTCCAGATGACCAAATTGGCTACTAC
CGAAGAGCTACCAGACGAATTCGTGGTGGTGACGGTAAAATGAAAGATCT
CAGTCCAAGATGGTATTTCTACTACCTAGGAACTGGGCCAGAAGCTGGAC
TTCCCTATGGTGCTAACAAAGACGGCATCATATGGGTTGCAACTGAGGGA
GCCTTGAATACACCAAAAGATCAC-3′
(SEQ ID No. 11)
Detection probe oligonucleotide
5′-GCATCATATGGGTTGCAACTGAGGGAGCCTTGAATACACCAAAAGAT
CAC-3′
(SEQ ID No. 12)
B-5′-GCATCATATGGGTTGCAACTGAGGGAGCCTTGAATACACCAAAAG
ATCAC-3′
(SEQ ID No. 12)
Capture oligonucleotide probe sequence
5′-GCGTTCCAATTAACACCAATAGCAGTCCAGATGACCAAATTGGCTAC
TAC-3′
(SEQ ID No. 13)
5′-GCGTTCCAATTAACACCAATAGCAGTCCAGATGACCAAATTGGCTAC
TAC-3′-P/N
(SEQ ID No. 13)

HIV specific capture and detection oligonucleotide probe sequences (HIV Set 1) and a possible corresponding target nucleic acid molecule are listed in Table 6. Other sets (HIV Sets 2 to 4) of HIV specific capture and detection oligonucleotide probe sequences and possible corresponding target nucleic acid molecules are listed in Tables 7 to 9 respectively. The bolded sequences in the target nucleic acid molecule sequences corresponding to the overlap with the capture and detection oligonucleotide probe sequences.

TABLE 6
HIV Set 1: HIV Specific Capture and Detection
Oligonucleotide Probes and Target Nucleic
Acid Molecule

Capture oligonucleotide probe
5′-CTTTCCGCTGGGGACTTTCCAGGGAGGCGTGGCCTGGGCGGG
ACTGC-3′
(SEQ ID NO. 14)
5′-CTTTCCGCTGGGGACTTTCCAGGGAGGCGTGGCCTGGGCGGG
ACTGC-3′-P/N
(SEQ ID No. 14)
Target nucleic acid molecule
5′-
CAGTCCCGCCCAGGCCACGCCTCCCTGGAAAGT
CCCCAGCGGAAAG
TCCCTTGTAGCAAGCTCGATGTCAGCAGTTCTT
GAAGTACTCCGGAT-3'
(SEQ ID NO. 15)
Detection oligonucleotide probe
5′-GATCCGGAGTACTTCAAGAACTGCTGACATCGAGCTTGCTAC
AAG-3′
(SEQ ID NO. 16)
b-5′-GATCCGGAGTACTTCAAGAACTGCTGACATCGAGCTTGCT
ACAAG-3′
(SEQ ID NO. 16)

TABLE 7
HIV Set 2: HIV Specific Capture and Detection
Oligonucleotide Probes and Target Nucleic
Acid Molecules

Capture oligonucleotide probe
5′-AAGTTCTTCTGATCCTGTCTGAAGGGATGGTTGTAAATGCCC
TATTATTC-3′
(SEQ ID No 17)
5′-AAGTTCTTCTGATCCTGTCTGAAGGGATGGTTGTAAATGCCC
TATTATTC-3′-P/N
(SEQ ID No 17)
Target nucleic acid molecule
5′-
GAATAATAGGGCATTTACAACCATCCCTTC
AGACAGGATCAGAAGAACTT
AAATCATTATATAATTTAGTAGCAGTCCTT
TATTGTTATTGTGTGCATCAAAGGATAGAG
GTAAAAGACACCAATG-3′
(SEQ ID No 18)
Detection oligonucleotide probe
5′-CATTGGTGTCTTTTACCTCTATCCTTTGATGCACACAATAAC
AATAAAGG-3′
(SEQ ID No 19)
b-5′-CATTGGTGTCTTTTACCTCTATCCTTTGATGCACACAATA
ACAATAAAGG-3′
(SEQ ID No 19)

TABLE 8
HIV Set 3: HIV Specific Capture and Detection
Oligonucleotide Probes and Target Nucleic
Acid Molecules

Capture oligonucleotide probe
5′-TGGGGTGGCCCCTTCTGATAATGCTGTAAACATGGGTATTAC
TTCTGGGC-3′
(SEQ ID No 20)
5′-TGGGGTGGCCCCTTCTGATAATGCTGTAAACATGGGTATTAC
TTCTGGGC-3′-P/N
(SEQ ID No 20)
Target nucleic acid molecule
5′-
GCCCAGAAGTAATACCCATGTTTACAGCAT
TATCAGAAGGGGCCACCCCA
CAAGATTTAAACACCATGTTAAACACAGTGGGGGGACATCAA
GCAGCCATGCAAATGTTAAAAGAGACCATC
AATGAGGAAGC
TGCAGAATG -3′
(SEQ ID No 21)
Detection oligonucleotide probe
5′-CATTCTGCAGCTTCCTCATTGATGGTCTCTTTTAACATTTGC
ATGGCTGC-3′
(SEQ ID No 22)
b-5′-CATTCTGCAGCTTCCTCATTGATGGTCTCTTTTAACATTT
GCATGGCTGC-3′
(SEQ ID No 22)

TABLE 9
HIV Set 4: HIV Specific Capture and Detection
Oligonucleotide Probes and Target Nucleic
Acid Molecules

Capture oligonucleotide probe
5′-AATCCCAGGATTATCCATCTTTTATAGATTTCTCCTACTGGG
ATAGGTGG-3′
(SEQ ID No 23)
5′-AATCCCAGGATTATCCATCTTTTATAGATTTCTCCTACTGGG
ATAGGTGG-3′-P/N
(SEQ ID No 23)
Target nucleic acid molecule
5′-
CCACCTATCCCAGTAGGAGAAATCTATAAA
AGATGGATAATCCTGGGATT
AAATAAAATAGTAAGAATGTATAGCCCTACCAG
CATTCTGGACATAAGACAAGGACCAAAAGA
ACCCTTTAGAGACTATGTAG-3′
(SEQ ID No 24)
Detection oligonucleotide probe
5′-CTACATAGTCTCTAAAGGGTTCTTTTGGTCCTTGTCTTATGT
CCAGAATG-3′
(SEQ ID No 25)
b-5′-CTACATAGTCTCTAAAGGGTTCTTTTGGTCCTTGTCTTAT
GTCCAGAATG-3′
(SEQ ID No 25)

Shiga toxin-producing E. coli (STEC) specific capture and detection oligonucleotide probe sequences (STEC Sets 1 to 3) and a possible corresponding target nucleic acid molecule are listed in Tables 10 to 12 respectively. The bolded sequences in the target nucleic acid molecule sequences corresponding to the overlap with the capture and detection oligonucleotide probe sequences.

TABLE 10
STEC Set 1: STEC Specific Capture and Detection
Oligonucleotide Probes and Target Nucleic
Acid Molecules

Capture oligonucleotide probe
5′-TATATGTTCAAGAGGGGTCGATATCTCTGTCCGTATACTATT
TAACGAAG-3′
(SEQ ID No 26)
5′-TATATGTTCAAGAGGGGTCGATATCTCTGTCCGTATACTATT
TAACGAAG-3′-P/N
(SEQ ID No 26)
Target nucleic acid molecule
5′-
CTTCGTTAAATAGTATACGGACAGAGATAT
CGACCCCTCTTGAACATATA
TCTCAGGGGACCACATCGGTGTCTGTTATTAACCACAC
CCCACCGGGCAGTTATTTTGCTGTGGATAT
ACGAGGGCTTGATGTCTATC-3′
(SEQ ID No 27)
Detection oligonucleotide probe
5′-GATAGACATCAAGCCCTCGTATATCCACAGCAAAATAACTGC
CCGGTGGG-3′
(SEQ ID No 28)
b-5′-GATAGACATCAAGCCCTCGTATATCCACAGCAAAATAACT
GCCCGGTGGG-3′
(SEQ ID No 28)

TABLE 11
STEC Set 2: STEC Specific Capture and Detection
Oligonucleotide Probes and Target Nucleic
Acid Molecules

Capture oligonucleotide probe
5′-ATTCAGTATAACGGCCACAGTCCCCAGTATCGCTGATATATT
ATTAAAGG-3′
(SEQ ID No 29)
5′-ATTCAGTATAACGGCCACAGTCCCCAGTATCGCTGATATATT
ATTAAAGG-3′-P/N
(SEQ ID No 29)
Target nucleic acid molecule
5′-
CCTTTAATAATATATCAGCGATACTGGGGA
CTGTGGCCGTTATACTGAAT
TGCCATCATCAGGGGGCGCGT
TCTGTTCGCGCCGTGAATGAAGAGAGTCAA
CCAGAATGTCAGATAACTGG-3′
(SEQ ID No 30)
Detection oligonucleotide probe
5′-CCAGTTATCTGACATTCTGGTTGACTCTCTTCATTCACGGCG
CGAACAGA-3′
(SEQ ID No 31)
b-5′-CCAGTTATCTGACATTCTGGTTGACTCTCTTCATTCACGG
CGCGAACAGA-3′
(SEQ ID No 31)

TABLE 12
STEC Set 3: STEC Specific Capture and Detection
Oligonucleotide Probes and Target Nucleic
Acid Molecules

Capture oligonucleotide probe
5'-CAGCGACTGGTCCAGTATTCTTTCCCGTCAACCTTCACTGTA
AATGTGTC-3'
(SEQ ID No 32)
5'-CAGCGACTGGTCCAGTATTCTTTCCCGTCAACCTTCACTGTA
AATGTGTC-3'-P/N
(SEQ ID No 32)
Target nucleic acid molecule
5'-
GACACATTTACAGTGAAGGTTGACGGGAAA
GAATACTGGACCAGTCGCTG
GAATCTGCAACCGTTACTGCAAAGTGCTCA
GTTGACAGGAATGACTGTCACAATCAAATC
CAGTACCTGTGAATCAGGCT-3'
(SEQ ID No 33)
Detection oligonucleotide probe
5'-AGCCTGATTCACAGGTACTGGATTTGATTGTGACAGTCATTC
CTGTCAAC-3'
(SEQ ID No 34)
b-5'-AGCCTGATTCACAGGTACTGGATTTGATTGTGACAGTCAT
TCCTGTCAAC-3'
(SEQ ID No 34)

Alpha-hemolysin producing Staphylococcus aureus specific capture and detection oligonucleotide probe sequences (SAUREUS Set 1) and a possible corresponding target nucleic acid molecule are listed in Table 13. The bolded sequences in the target nucleic acid molecule sequences corresponding to the overlap with the capture and detection oligonucleotide probe sequences.

TABLE 13
SAUREUS Set 1: Alpha-Hemolysin producing
S. Aureus Specific Capture and Detection
Oligonucleotide Probes and Target Nucleic
Acid Molecule

Capture oligonucleotide probe
5′-CATGAAAAGTTGATTGCCATATACCGGGTTCCAAGAATCTCT
ATCATATG-3′
(SEQ ID No 35)
5′-CATGAAAAGTTGATTGCCATATACCGGGTTCCAAGAATCTCT
ATCATATG-3′-P/N
(SEQ ID No 35)
Target nucleic acid molecule
5′-
CATATGATAGAGATTCTTGGAACCCGGTAT
ATGGCAATCAACTTTTCATG
AAAACTAGAAATGGTTCTATGAAAGCAGCAGAGAACTTCCTTGA
TCCTAACAAAGCAAGTTCTCTATTATCTTC
AGGGTTTTCACCAGACTTCG-3′
(SEQ ID No 36)
Detection oligonucleotide probe
5′-CGAAGTCTGGTGAAAACCCTGAAGATAATAGAGAACTTGCTT
TGTTAGGA-3′
(SEQ ID No 37)
b-5′-CGAAGTCTGGTGAAAACCCTGAAGATAATAGAGAACTTGC
TTTGTTAGGA-3′
(SEQ ID No 37)

FOR TABLES 6, 7, 8 9 the PCR primers may be in the first 36 bases on the 5′ side or any flanking sequence that will amplify the target that will generate product of at least 100 bp or more optimally 150, 200 or 300 bp.
P/N denotes that the sequence can comprise a phosphate end (as found in nucleotides) or an amine for example for attachment to a solid support.

### Example 11 PCR Detection of SARS-CoV2

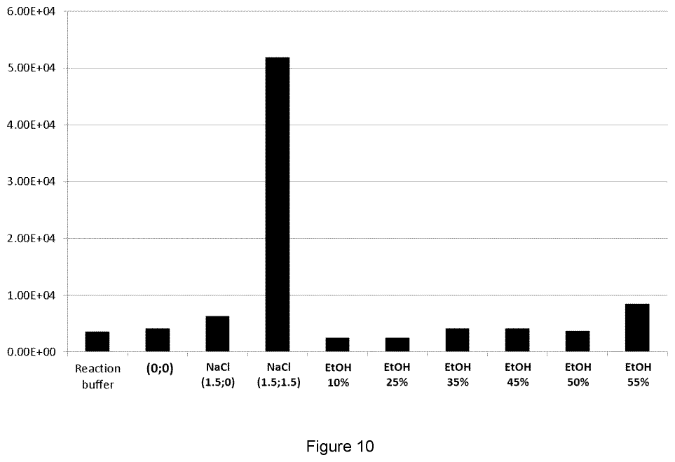

PCR reactions were initiated with 10 ng of template plasmid DNA (SARS-CoV2 nucleocapsid plasmid) with the following primer combinations:
1. Forward primer SEQ ID No 2 and reverse primer SEQ ID No 3 (PCR product of 138 bp);
2. Forward primer SEQ ID No 2 and reverse primer SEQ ID No 8 (PCR product of 279 bp);
3. Forward primer SEQ ID No 7 and reverse primer SEQ ID No 3 (PCR product of 236 bp); and
4. Forward primer SEQ ID No 7 and reverse primer SEQ ID No 8 (PCR product of 377 bp).
   The expected length of the corresponding PCR product of each of the primer combinations can be calculated using the nucleocapsid gene sequence of SARS-CoV2 as shown in Table 1. The expected lengths of PCR products are listed in parentheses above. The PCR reactions were run for 35 cycles, with lid temperature of 105° C., in a reaction volume of 100 μl, melting temperature of 94° C. (30 sec), annealing temperature of 55° C. (30 sec), extension temperature of 72° C. (1 min). The PCR products were separated by a 16% TBE polyacrylamide gel run at 100 volts alongside a molecular weight marker. The resulting gel is shown in FIG. 10. The gel showed that in each primer combination, a PCR product of corresponding to the expected length was observed, confirming that the PCR reactions successfully amplified the desired sequences in the SARS-CoV2 nucleocapsid gene.

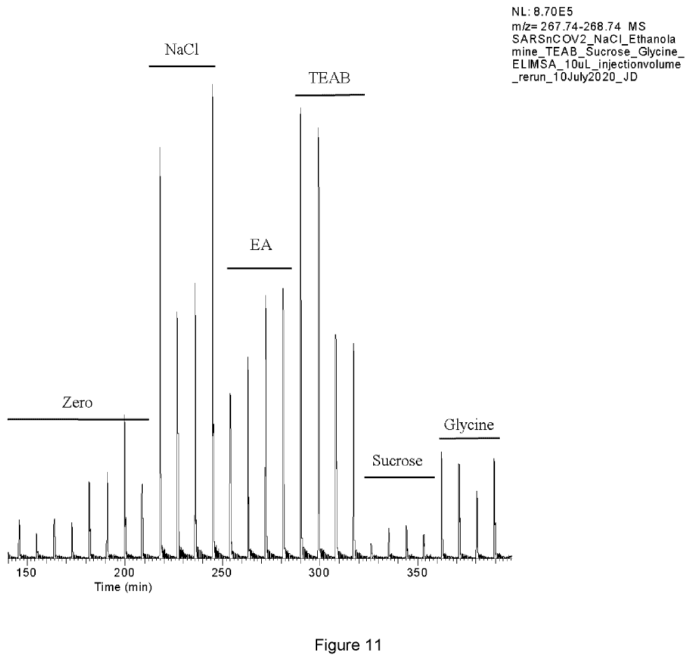

FIG. 11 shows PCR results using plasmid containing SARS-CoV2nucleocapsid gene as template and SARS-CoV2 set 1 PCR primers (SEQ ID Nos 2 and 3). Different amounts of template was used including 0 template, trace detection amount of template and different amounts of template (0.1 ng, 1 ng, 10 ng, 50 ng). FIG. 13 also shows PCR product produced using different concentrations of Mg (2 mM, 2.5 mM, 3.0 mM, 3.5 mM, or 4.0 mM) in lanes 6 to 10. PCR was run for 35 cycles, with an annealing temperature of 55° C. using a hot start. Primers resuspended in 10 mM Tris, 0.1 mM EDTA to a final concentration of 0.2 uM in PCR rxn using Qiagen mastermix. Plasmid concentrations ranged from 100 μL PCR product separated by a 16% TBE polyacrylamide gel run at 100 volts. Largest amount of PCR product was produced with 50 ng of starting plasmid DNA and between 1.5 to 4 mM of Mg²⁺. Significant and observable amount of PCR product was obtained with trace detection amount of template (lane 1), subnanogram (lane 2) and nanogram amount (lane 3) of template DNA.

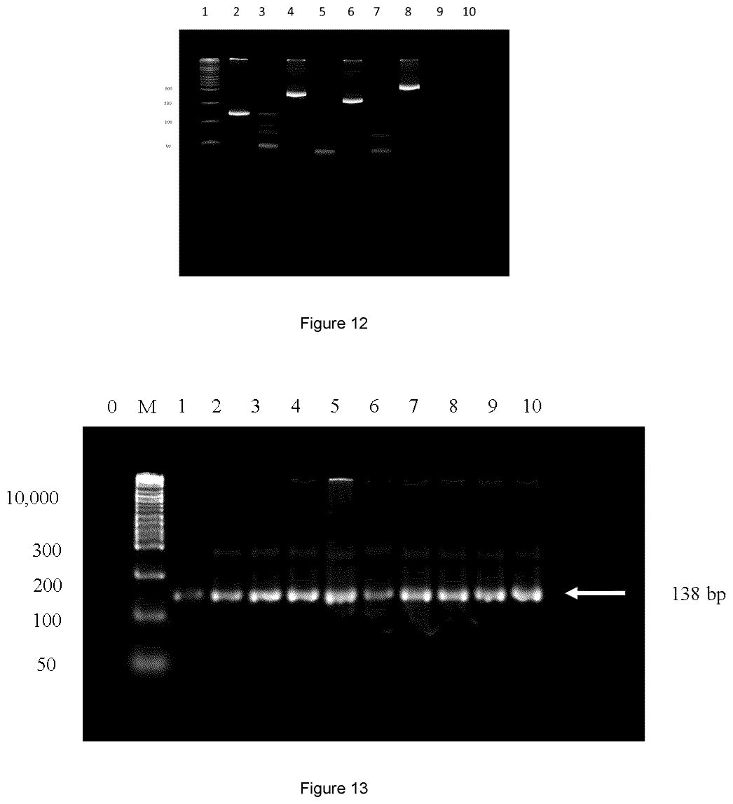

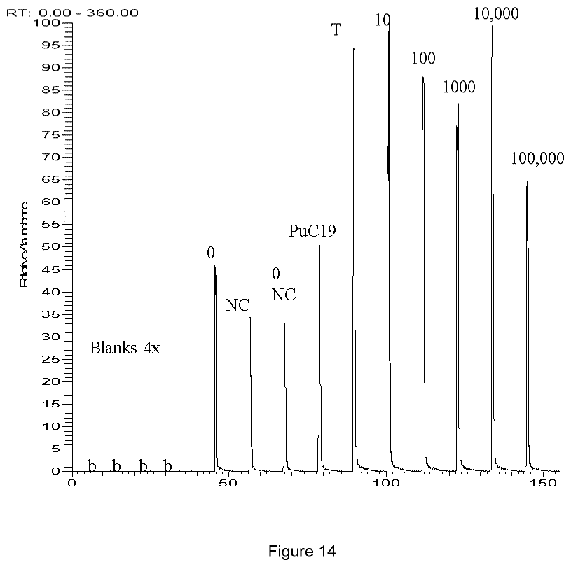

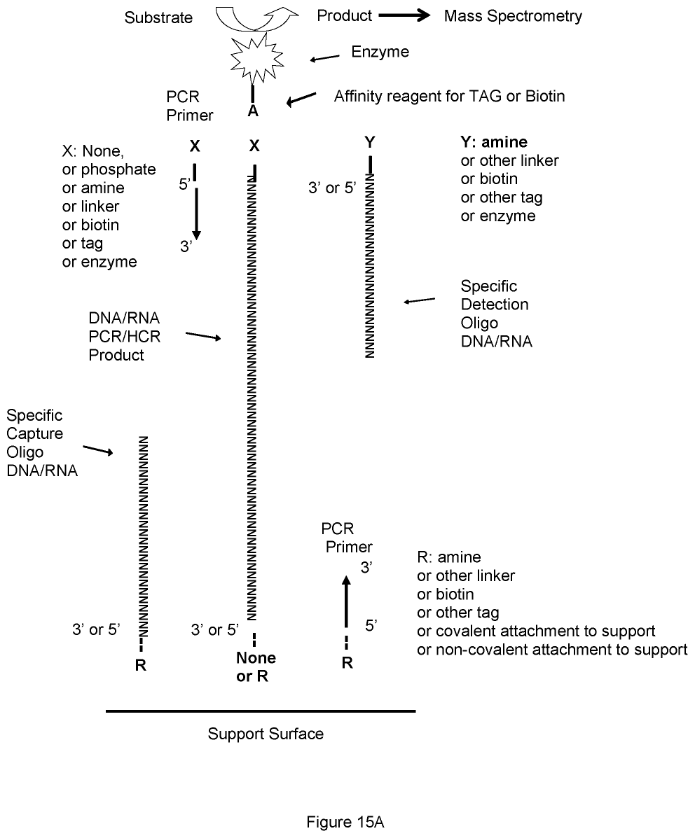

FIG. 12 shows results of detection of PCR product of SARS-CoV2 nucleocapsid gene using the DNA detection method of the present disclosure. Detection was performed with the capture oligonucleotide probe SEQ ID No. 6 with 3′ attached to solid support and the 5′-biotinylated detection oligonucleotide SEQ ID No. 5. Zero template DNA and PuC19 plasmid DNA were used as negative controls. Various amounts of template DNA ranging from trace, 10 fg, 100 fg, 1 pg, 10 pg, to 100 pg were used. MS signal was observed for all amounts of template DNA. No signal was observed for no template or Puc19 plasmid. The amplified target nucleic acid product is 138 nt in length of SEQ ID No. 4.

The methods described herein can be applied to detect viral target nucleic acid molecule through highly selective hybridization method when the capture oligos are immobilized to the solid state and with independent biotinylated detection oligonucleotide probes for secondary enzyme amplification by the reaction of AMP with APSA. As shown herein viral DNA can be detected using various immobilization methods of Capture oligonucleotide probe and followed by selective hybridization and APSA amplification, including: Capture oligonucleotide probe non-covalently bound to PVDF membrane, Capture oligonucleotide probe 3′ coupled to polystyrene beads in a 96 well filter plate, Capture oligonucleotide probe covalently immobilized to 96 well reactive plates, Capture oligonucleotide probe covalently immobilized to 96 well polystyrene plates through polylysine coating, and to cover glass (SiO2) by amino silylation and crosslinking.

### Example 12 Preparation of Oligonucleotide Probes

It can be appreciated that the oligonucleotide probes of the present disclosure may be prepared according methods known to a person skilled in the art or may be purchased from existing commercial sources.

For example, capture oligonucleotides may be presented on silica, polystyrene, agarose, melamine, PVDF, or other supports. The silica, polystyrene, agarose, melamine, PVDF, or other supports can be in the form of microparticles or nanoparticles. Optionally, the silica, polystyrene, agarose, melamine, PVDF, or other supports can be a 2-dimensional surface, a 3-dimensional surface, or a 1-dimensional fibre or filament.

For example, silica microparticle or nanoparticle may be functionalized to produce reactive sites for attachment of oligonucleotides. For example, silica microparticle or nanoparticle may be functionalized with an amine group using 3-aminopropyltrimethoxysilane or with an epoxide group with 3′ glycidoxy propyltrimethoxysilane. In this case, the amine or the epoxide can serve as reactive sites for attachment of oligonucleotides. Other reactive or functional sites include silanol, hydroxyl, carboxylic acid, anything that reacts with amine or carboxyl groups, maleimide, N-hydroxysuccinimide (NHS), N-oxysuccimide (NOS) H-hydroxysuccinimide (NHS), N-oxysuccinimide (NOS), maleimide, hydrazide, glutaraldehyde coupling, or PEG cross-linking

etc. Although typical for the capture or primer to comprise an amine group and the solid support to comprise a group that can react therewith, other configurations can be used. For example, a NHS group or other NOS linker can be added to the capture oligonucleotide or primer to be attached to the solid surface and the solid surface can comprise a functionalizable amine.

For example, the oligonucleotide may be attached to reactive sites one nucleotide at a time. For example, a first nucleotide may be attached via the C1 position, the 3′-OH group or the 5′-OH group. Optionally, the first nucleotide may be attached to the solid support via a linker. For example, amine oligonucleotides may be attached to carboxyl groups, such as carboxylic acid groups, optionally through activated esters thereof. For example, the first nucleotide may be attached via the 3′-OH position and the 5′-OH may be protected with dimethyltrityl (DMT) group.

For example, the oligonucleotide may be attached in portions of oligomers of nucleotides, or it may be attached as one oligonucleotide.

The oligonucleotide may be synthesized through conventional nucleotide synthesis methods known to persons skilled in the art. During organic synthesis, the synthetic intermediates can be protected using conventional protective groups known to persons skilled in the art. For example, the nucleotide base may be protected using benzoyl group or isobutyryl group. Additionally, during organic synthesis, functional groups may be modified to increase their reactivity using methods known to persons skilled in the art. For example, carboxylic acids can be activated through activated esters such as succinimide esters. For example, thiol, mecapto or sulphide or SH-oligonucleotides may be covalently linked via an alkylating agent such as iodoacetamide.

It can be appreciated that an oligonucleotide can be attached covalently to an enzyme by methods known to persons skilled in the art. For instance, the detection oligonucleotide may be attached covalently to the detection enzyme, such as APSA.

For example, proteins, peptides, enzymes, DNA, RNA or antibodies, oligomers or polymers may be coupled or cross linked primary amines (—NH2) found in N-terminus and many amino acids, carboxyls (—COOH at the C-terminus of each polypeptide chain and in the side chains of aspartic acid (Asp, D) and glutamic acid (Glu, E), Sulfhydryls (—SH) in the side chain of cysteine (Cys, C) and Carbonyls (—CHO) such as Ketone or aldehyde groups can be created in glycoproteins by oxidizing the polysaccharide post-translational modifications (glycosylation) with sodium meta-periodate. For example, NHS-activated acid may couple to a carboxylic acid in the presence of organic base in an anhydrous solvent. A coupling reagent such as dicyclohexylcarbodiimide (DCC) or ethyl(dimethylaminopropyl) carbodiimide (EDC) is then added to form a stable bound with a primary amine.

Optionally, cross-linking agents may be used. For example, mono, bifunctional or multifunctional cross-linking reagents may be used. For example, NHS, sulfo-NHS, DSS, BS3 (sulfo-DSS), amine-to-amine cross-linkers may be used. For example, water-soluble analog sulfo-NHS, hydroxybenzotriazole (HOBt), 1-hydroxy-7-azabenzotriazole (HOAt), and pentafluorophenol may all be used as linking reagents for nucleic acids, peptides and proteins or antibodies. For example, maleimide may be used for cross-linking thiol groups in for example cysteine.

It can be appreciated by a person skilled in the art that protein, peptides and nucleic acids present primary amines and/or hydroxyl groups, and may be modified or cross-linked through the primary amines and/or hydroxyl groups.

For example, Sulfo-SMCC (sulfosuccinimidyl 4-(N-maleimidomethyl)cyclohexane-1-carboxylate), EDC 1-Ethyl-3-(3-dimethylaminopropyl) carbodiimide, Sulfo-NHS (N-hydroxysulfosuccinimide), BS3 (bis(sulfosuccinimidyl)suberate), DST (disuccinimidyl tartrate), SPDP (succinimidyl 3-(2-pyridyldithio)propionate) may be used as cross-linking agents. For example, dithiobis succinimidyl propionate can be used to cross-link amine to amine. For example, BMH bismaleimidohexane can be used to cross-link sulfhydryls to sulfhydryls, such as in cysteine residues in proteins or peptides.

For example, sulfo-EGS (ethylene glycol bis(sulfosuccinimidyl succinate)) can be used to cross-link amines to amines.

For example, SM(PEG)4 (PEGylated SMCC crosslinker) can be used to crosslink amines to sulfhydryl. These crosslinkers containing NHS-ester and maleimide groups at ends of water-soluble polyethylene glycol spacer arms (17.6 to 95.2 A).

For example, Sulfo-EMCS (N-ε-maleimidocaproyl-oxysulfosuccinimide ester) can be used to crosslink amines to sulfhydryls.

For example, Sulfo-SMPB (sulfosuccinimidyl 4-(N-maleimidophenyl)butyrate) can be used to crosslink amines to sulfhydryls.

It can be appreciated that a protein, a peptide, or an amine-containing molecule can be biotinylated using methods known to persons skilled in the art. For example, NHS-PEG4-Biotin N-Hydroxysuccinimide (NHS) is a pegylated, water-soluble reagent for biotin labeling.

Optionally, a linker may be used between an oligonucleotide and a protein such as an enzyme.

For example, the derivatization of the 1% cross-linked polystyrene resin may be performed according to the procedure of Horiki et al. (15).

For example, Polystyrene Carboxyl Resin: may be prepared by the method of Bayer et al. (16).

For example, Cyanogen bromide activation of Sephacryl S-500 may be performed as described by Biinemann (8).

For example, Chondroitin Sulfate-Coated CPG Supports: may be prepared by CPG long-chain alkylamine of chondroitin sulfate (type A or type C) with EDC (Ghosh Musso NAR 1987).

For example, oligonucleotides may be prepared by blocking with 5′-aminohexyl and 5′-Cystaminyl Phosphoramidate or other derivatives of oligonucleotides. For example, reaction of the 5′-phosphorylated oligonucleotides with 1,6-diaminohexane in the presence of 0.1 M EDC in 0.1 M N-methylimidazole, pH 6.0 may be carried out according to the direct coupling protocol described by Chu et al. (20)

For example, oligonucleotides may be attached to N-Hydroxysuccinimide-activated using N-hydroxysuccinimide-activated carboxyl Sephacryl support with 5′-aminohexyl or 5′-cystaminyl phosphoramidate or other protected oligonucleotide in 0.2 M HEPES, pH 7.7.

### Example 13 DNA Detection at Attomolar Concentration

DNA detection assay was performed according to a method of DNA detection (Example 5) as described herein. HIV DNA was used as a target. The target nucleic acid molecule has sequence of SEQ ID No.15. The capture oligonucleotide probe of SEQ ID No.14 was used, with 3′ attached to polystyrene as solid support. The detection oligonucleotide probe of SEQ ID No. 16 was used with 5′ being biotinylated. Different concentrations of the target nucleic acid molecule were used: 0 attomolar (negative control), 1 attomolar, 2 attomolar, 3 attomolar, 4 attomolar, 5 attomolar, and 6 attomolar. The hybridization of capture and detection oligonucleotide probes to the target nucleic acid molecule was done in presence of NaCl. The solid support (polystyrene bead) was washed with buffer containing NaCl, and separated by centrifugation. The APSA enzyme reaction was performed in presence of NaCl. The reaction mixture was treated with C18 reverse phase chromatography using 70% acetonitrile in water as the mobile phase. 1 μL of the reaction product was injected on MS. MS detection was done at m/z=268.

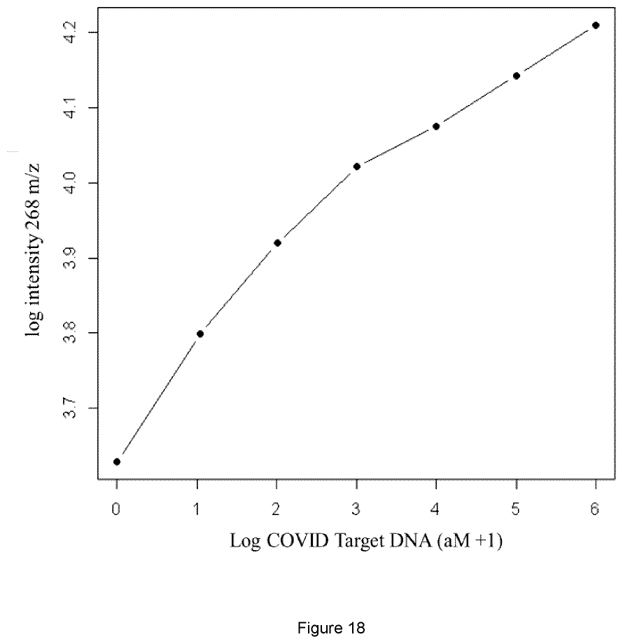

FIG. 18 shows a standardization curve of concentration of DNA target nucleic acid molecule vs MS signal intensition. The results show that the DNA detection assay is sensitive at the attomolar range.

### Example 14 DNA Detection at Attomolar Concentration

DNA detection assay was performed according to a method of DNA detection (Example 5) as described herein. SARS-CoV2 DNA was used as a target. The target nucleic acid molecule has sequence of SEQ ID No.11. The capture oligonucleotide probe of SEQ ID No.13 was used, with 3′ attached to polystyrene as solid support. The detection oligonucleotide probe of SEQ ID No. 12 was used with 5′ being biotinylated. Different concentrations of the target nucleic acid molecule were used from 1 picomolar to 1 micromolar (0.1 attomolar, 1 attomolar, 10 attomolar, 100 attomolar, 1 femtomolar, 10 femtomolar, 100 femtomolar). Tris buffer was used as blank negative control for MS detection. Zero DNA target nucleic acid was used as negative control for the detection assay. The hybridization of capture and detection oligonucleotide probes to the target nucleic acid molecule was done in presence of NaCl. The solid support (polystyrene bead) was washed with buffer containing NaCl, and separated by centrifugation. The APSA enzyme reaction was performed in presence of NaCl. The reaction mixture was treated with C18 reverse phase chromatography using 95% acetonitrile in water, 0.1% acetic acid as the mobile phase, 20 μL/min. 1 μL of the reaction product was injected on MS. MS detection was done at m/z=268.

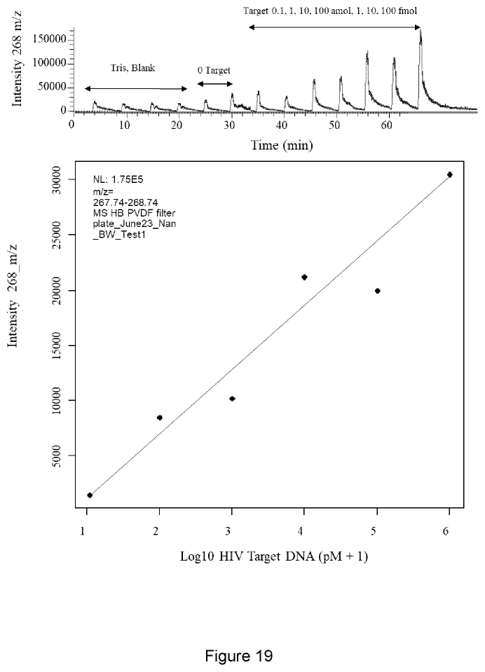

FIG. 19 shows a standardization curve of concentration of DNA target nucleic acid molecule vs MS signal intensition. The results show that the DNA detection assay is sensitive at picomolar to micromolar range.

### Example 15 Detection of Low Concentration Synthetic DNA Targets

Synthetic DNA, PCR products and plasmid DNA were each used as target nucleic acid molecules. PCR products and plasmid DNA assays are described further in Example 16. Synthetic DNA, as used in these examples refers to single stranded DNA that is synthesized. PCR products refer to amplified DNA (optionally starting from RNA) and plasmid DNA comprises the target of interest in the context of a larger plasmid.

The PCR primers, oligonucleotide capture and detection probes designed in Example 10 were used in this Example and in Example 16. HIV, COVID, Shiga-toxin producing E. coli (STEC), and hemolysin DNA from synthetic targets and HIV and COVID plasmids were detected using various methods of the present disclosure as described below.

The capture oligonucleotide pobe was immobilized on a NOS surface chemistry 96 well polystyrene reactive plate.

In general, the capture oligonucleotide probe in surface binding buffer (10 mM Na₂PO₄+1 mM EDTA buffer, pH 8.5) was added to the plate and incubated at 4° C. overnight. The wells were then washed 3 times with additional surface binding buffer, and quenched and blocked with 3% BSA for 1 h. The quenched and blocked plate was washed 3 times with 20 mM Tris pH8.00+1 mM EDTA followed by 3 times with Binding Buffer (20 mM Tris pH8.00+1M NaCl+1 mM EDTA)

HIV, COVID, Shiga and hemolysin DNA from synthetic targets and the HIV and COVID plasmids were detected using capture oligonucleotide probes described herein.

The appropriate capture oligonucleotide probe was immobilized at the 3′-end or the 5′ end via an amine functionality, as mentioned on NOS surface chemistry 96 well polystyrene reactive plates. Each experiment was done in triplicates (n=3).

The synthetic (results described below), PCR or plasmid viral DNA (results described in Example 16) (e.g. the target nucleic aid molecule) and the corresponding detection oligonucleotide probe were added to each well of the plate (comprising the capture oligonucleotide probe) to start DNA hybridization for around 1.5 h. The wells were then washed 3 times with Binding Buffer (20 mM Tris pH8.00+1 M NaCl+1 mM EDTA). The plate was blocked with 1% BSA for 5 min and then incubated with APSA solution in 1% BSA for 15 min, and washed 11 times with designated buffers (6× quick wash with Binding Buffer (20 mM Tris pH8.00+1 M NaCl+1 mM EDTA), 3×5 min with 20 mM Tris pH8.00+1 M NaCl (no EDTA), and 2× with 20 mM Tris pH8.00+2M AMBIC (1×5 min and 1×15 min). The plate was then incubated with 1 mM AMP for 2 h before collecting the assay products. The collected samples were analyzed using mass spectrometry (m/z 136).

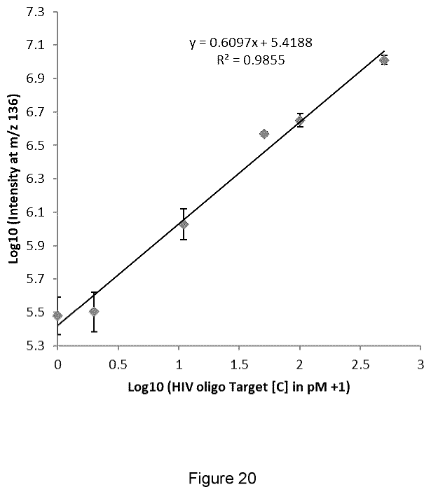

FIGS. 20, 21, 22 and 23 show results where single stranded synthetic DNA targets.

### HIV Oligonucleotide Target

FIG. 20 shows a graph of MS signal intensity at m/z 136 at different concentrations of HIV synthetic DNA target nucleic acid molecule (1 μM to 500 μM).

The HIV DNA sequences used are shown in Table 9. The target nucleic acid molecule has sequence of SEQ ID No. 24. The capture oligonucleotide probe of SEQ ID No. 23 was used, with 3′ attached through an amine functionality to NOS-surfaced polystyrene as solid support. The detection oligonucleotide probe of SEQ ID No. 25 was used with 5′ being biotinylated.

FIG. 20 shows that the detection method described herein was able to detect HIV DNA target nucleic acid molecule at concentrations as low as 1 μM. Injecting 1 uL of a 1 μM solution corresponds to detection at the atto mol amount. This level of detection is achieved without PCR.

### SARS-CoV 2 (COVID) DNA Oligonucleotide Target Detection

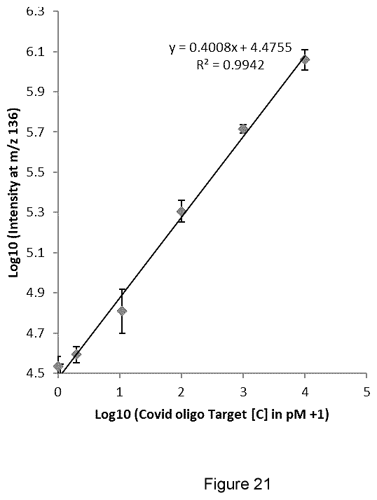

FIG. 21 shows a graph of MS signal intensity at m/z 136 at different concentrations of SARS-CoV 2 synthetic target nucleic acid molecule (100 fM to 10 nM). The method used herein was able to detect SARS-CoV 2 target oligonucleotide at concentrations as low as 100 fM. The sequence detected is part of the nucleocapsid phosphoprotein.

The SARS-Co-V 2 DNA sequences used in the assay in FIG. 21 are shown in Table 3. The target nucleic acid molecule has sequence of SEQ ID No. 4. The capture oligonucleotide probe of SEQ ID No. 6 was used, with 3′ attached through an amine functionality to NOS-surfaced polystyrene as solid support. The detection oligonucleotide probe of SEQ ID No. 5 was used with 5′ being biotinylated

Shiga-Toxin Producing E. coli (STEC) DNA Detection

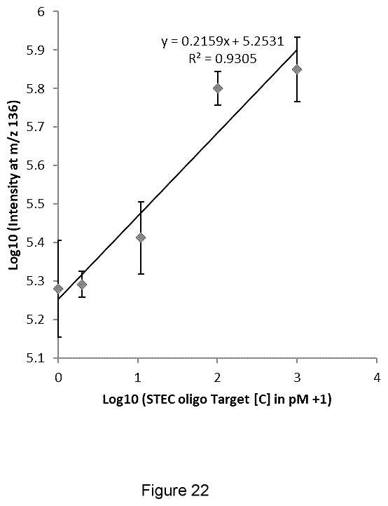

FIG. 22 shows a graph of MS signal intensity at m/z 136 at different concentrations of synthetic STEC target nucleic acid molecule (1 pM to 1 nM). The method used herein was able to detect STEC target nucleic acid molecule at concentrations as low as 1 pM.

The STEC DNA sequences used are shown in Table 11. The target nucleic acid molecule has sequence of SEQ ID No. 30. The capture oligonucleotide probe of SEQ ID No. 29 was used, with 3′ attached through an amine functionality to NOS-surfaced polystyrene as solid support. The detection oligonucleotide probe of SEQ ID No. 31 was used with 5′ being biotinylated.

S. aureus Hemolysin DNA Detection

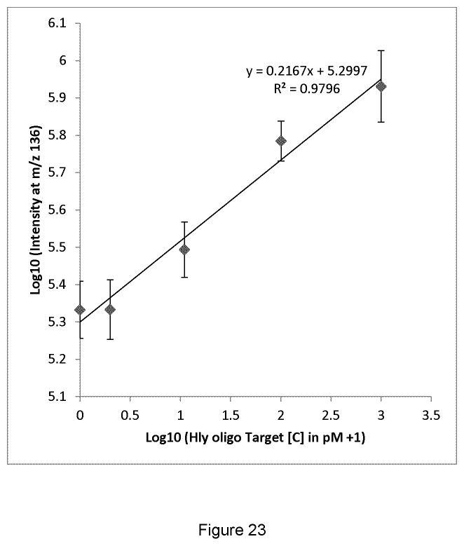

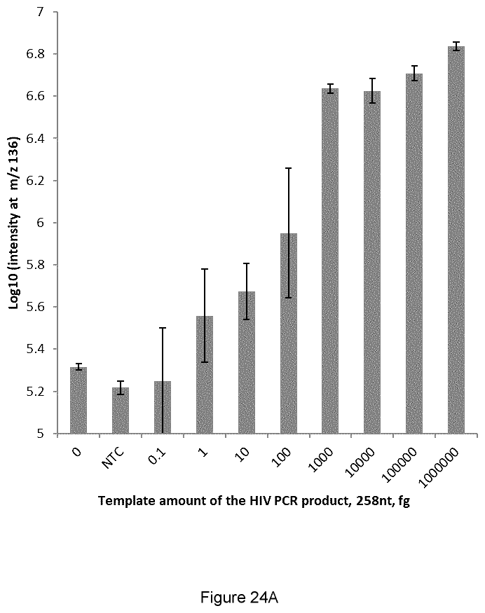

FIG. 23 shows a graph of MS signal intensity at m/z 136 at different concentrations of hemolysin synthetic target nucleic acid molecule (1 pM to 1 nM). The method used herein was able to detect hemolysin target nucleic acid molecule at concentrations as low as 1 Pm (e.g. 1 uL of a 1 pM solution was detectable or 1 attomole).

The hemolysin DNA sequences used are shown in Table 13. The target nucleic acid molecule has sequence of SEQ ID No. 36. The capture oligonucleotide probe of SEQ ID No. 35 was used, with 3′ attached through an amine functionality to NOS-surfaced polystyrene as solid support. The detection oligonucleotide probe of SEQ ID No. 37 was used with 5′ being biotinylated.

### Example 16 Detection of Low Concentration PCR and Plasmid DNA Targets

Example 15 provides some details on the detection of PCR and plasmid DNA products further described here.

For PCR target, the target nucleic acid was prepared by amplification of a plasmid using a PCR reaction. An agarose gel was run visualizing the PCR products amplified from the HIV plasmid and for sensitivity comparison to methods described herein.

As described and shown here, the described methods using mass spectrometry resulted in enhanced sensitivity.

PCR reactions were run with 1 ng to 1 attogram (rep1 & rep2) as template. Reactions initiated with 1 μl HIV plasmid DNA. PCR 35 cycles, lid temp 105° C., 25 μL reaction volume, 94° C. melting (30 s), 58° C. annealing (30 s), 72° C. extension (30 s). The products were separated by a 2% Agarose gel run at 100 volts for 2 hour big gel tank, ladder runs straight). 5 μl sample loaded. 3 μl ladder loaded. InGel staining with GelRed™.

In experiments using PCR template, 5 μl of the PCR sample (same as loaded on gel) was also subjected to hybridation steps described herein.

In experiments using plasmid, plasmid was either attached via NOS to PVDF in a 96 well format or adsorbed thereon.

Further details are provided below.

### HIV DNA Detection

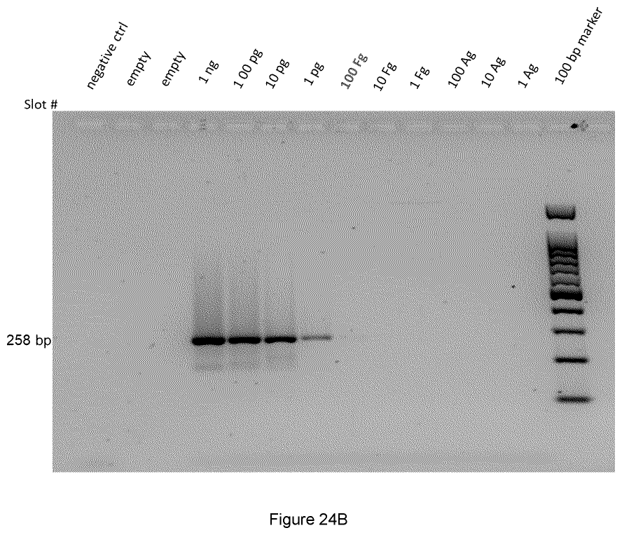

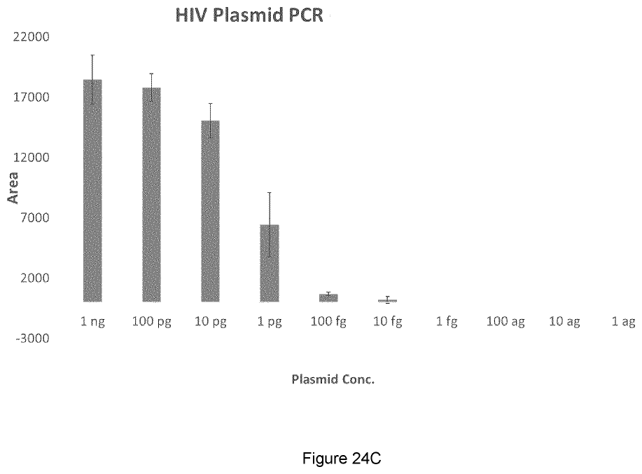

An HIV Gag Pr55 coding plasmid (e.g. Accesion number GQ432554.1) was used in an assay comparing primers that hybridize within the capture oligonucleotide probe region and primers that hybridize outside the probe region of the capture oligonucleotide probe. Two sets of primers generating two PCR products of different length, one of 133 bp fragment (primers within region of capture probe) and one of 258 bp fragment (primers outside region of capture probe), were used and the PCR products were used as target nucleic acid molecule (e.g. PCR template). The assay involved the use of a capture probe and detection probe (e.g. full sandwich method) and was compared to the sensitivity PCR amplified plasmid as shown by gel and quantified using image analysis. See FIGS. 24A, B and 24C.

PCR reactions were run with atto, femto, pico or nano gram amounts of the template HIV plasmid DNA. Reactions initiated with 1 μl HIV plasmid DNA. PCR 35 cycles, lid temp 105° C., 25 μL reaction volume, 94° C. melting (30 s), 58° C. annealing (30 s), 72° C. extension (30 s). The PCR products were used (either for gel visualization or for use in the assay described and mass spec analysis) without purification (e.g. 5 μl aliquot of reaction used directly).

The capture oligonucleotide probe (SEQ ID No. 23), and detection oligonucleotide probe (SEQ ID No. 25) for the 258 bp target are shown in Table 9. The forward primer used to generate the PCR product had sequence 5′-CCAGGCCAGATGAGAGAACC-3′ (SEQ IC No. 38). The reverse primer used to generate the PCR product had sequence 5′-TGAAGCTTGCTCGGCTCTTA-3′ (SEQ ID No. 39). The 258 target nucleic acid molecule has sequence:

(SEQ ID No. 40)
5′-
CCAGGCCAGATGAGAGAACCAAGGGGAAGTGACATAGCAGGAACTACTAG
TACCCTTCAGGAACAAATAGGATGGATGACAAATAATCCACCTATCCCAG
TAGGAGAAATTTATAAAAGATGGATAATCCTGGGATTAAATAAAATAGTA
AGAATGTATAGCCCTACCAGCATTCTGGACATAAGACAAGGACCAAAAGA
ACCCTTTAGAGACTATGTAGACCGGTTCTATAAAACTCTAAGAGCCGAGC
AAGCTTCA-3′.

For the 258 bp PCR product target, the PCR primers were outside or out flanked the capture and detection sequences. The detection was done as described below.

5 μL of the PCR sample solution, diluted into 100 μL with Binding Buffer, was used for each “DNA ELiMSA reaction” e.g. for the incubating with a capture and detection probes, for incubating with the reporter enzyme detection probe and substrate and for mass spec analysis). The capture oligonucleotide probe was covalently immobilized on NOS surface chemistry 96 well polystyrene reactive plates (n=3) through a 3′ amine on the capture oligo probe. The capture oligonucleotide probe (also referred to as Capture DNA) in Surface Binding Buffer (10 mM Na2PO4+1 mM EDTA buffer, pH 8.5) was added to the plate and incubated at 4° C. overnight. Washed 3 times with Surface Binding Buffer, and then quenched and blocked the plate with 3% BSA for 1 h and washed 3 times with 20 mM Tris pH8.00+1 mM EDTA followed by 3 times with Binding Buffer (20 mM Tris pH8.00+1 M NaCl+1 mM EDTA). Target nucleic acid molecule and detection Probe DNA was added to each well of the plate to start DNA hybridization for around 1.5 h, and washed 3 times with Binding Buffer (20 mM Tris pH8.00+1 M NaCl+1 mM EDTA). The plated was blocked with 1% BSA for 5 min and then incubated with APSA solution in 1% BSA for 15 min, and washed 10 times with designated buffers (6× quick wash with Binding Buffer (20 mM Tris pH8.00+1M NaCl+1 mM EDTA), 3×5 min with 20 mM Tris pH8.00+1M NaCl (no EDTA), and 2× with 20 mM Tris pH8.00+2M AMBIC (1×5 min and 1×15 min). The plate was then incubated with 1 mM AMP for 2 h before collecting the assay products. NTC represents the no-template-control of the PCR reaction. Error bar=STDEV

FIG. 24A shows the mass spectrometry detection of the 258 nt PCR product from femto gram amounts of the HIV plasmid DNA sequence where the PCR primers were outside or out flanked the capture and detection sequences. As mentioned crude PCR product was used in the assay.

FIG. 24B shows an agarose gel demonstrating that only 100 fg is faintly visible. FIG. 24C quantifies the amount seen on the gel in FIG. 24B.

The capture oligonucleotide probe (SEQ ID No. 23), and detection oligonucleotide probe (SEQ ID No. 25) for the 133 bp target were the same as for the 258 np PCR product and are shown in Table 9. The forward primer used to generate the PCR product had sequence 5′-CCACCTATCCCAGTAGGAGAAATCTATAAAAGATGG-3′ (SEQ IC No. 41). The reverse primer used to generate the PCR product had sequence 5′-CTACATAGTCTCTAAAGGGTTCTTTTGGTCCTTGTC-3′ (SEQ ID No. 42). The target nucleic acid molecule has sequence:

(SEQ ID No. 43)
5′-
CCACCTATCCCAGTAGGAGAAATTTATAAAAGATGGATAATCCTGGGATT
AAATAAAATAGTAAGAATGTATAGCCCTACCAGCATTCTGGACATAAGAC
AAGGACCAAAAGAACCCTTTAGAGACTATGTAG-3′.

### Covid Detection

Covid PCR product was also assayed using primers and probes described herein.

SARS-Co-V 2 target nucleic acid molecule PCR product was prepared by the following PCR conditions. The PCR reactions initiated with 10 ng of SARS-CoV-2 positive ctrl Plasmid. (35 cycles, lid temp 105° C., 50 μL reaction volume, 94° C. melting (30 s), 58° C. annealing (30 s), 72° C. extension (1 min)). 5 μl of the products were separated and 5 μl were subjected to assays described herein. Specifically, the 25 uL PCR reaction was then aliquoted 5 uL for GEL analysis and 5 uL for Hybridization and mass spectrometry analysis.

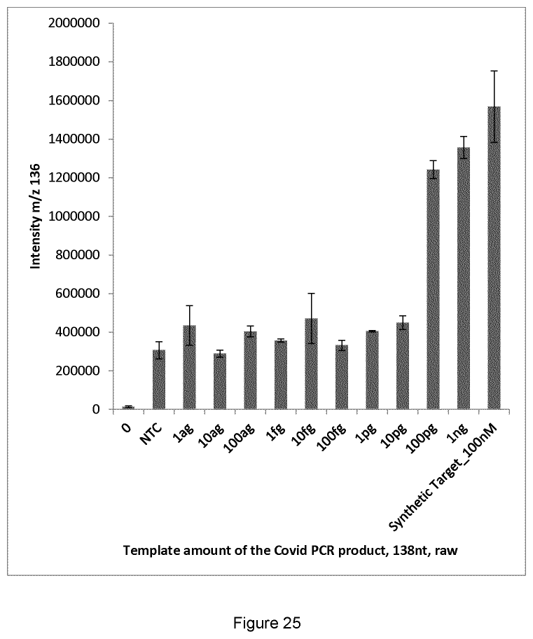

FIG. 25 shows the detection of the 138 nt PCR product from the Covid DNA plasmid DNA sequence where the PCR primers were within the capture and detection sequences.

The methods described are sensitive particularly considering that only a small volume of the total reaction volume is subjected to mass spectrometry analysis. Typically 1-2 μl of the 200 μl the final reaction volume was subjected to mass spectrometry. In each case, the “DNA EliMSA” described herein detected target with 10-100 times more sensitivity. As only 1/100 or 1/200 of the reaction volume was assayed by mass spectrometry, DNA EliMSA assays as described herein for the same level of template can be 10,000-20,000 times more sensitive.

PCR is considered to be very sensitive but can be labor intensive or time consuming as it typically involves manual gel loading, gel staining and quantification. The methods described can be automated. For example as the assays described herein can be performed in 96 well plates, 96 well injection robots can be used to automate.

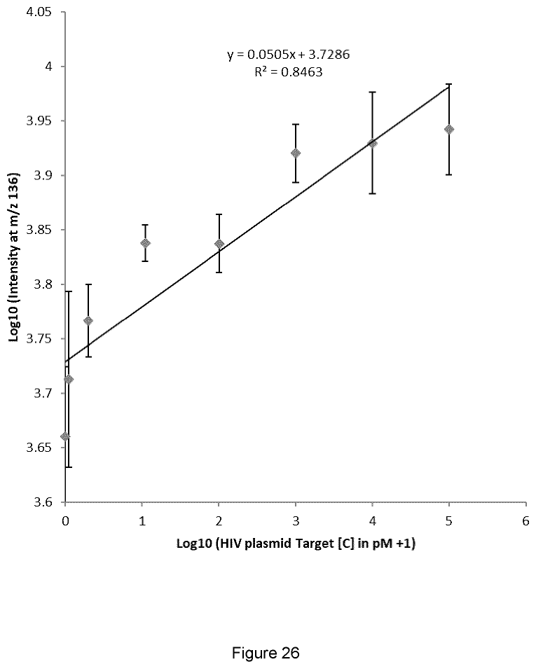

Direct detection of plasmid DNA was also demonstrated. FIG. 26 shows detection of HIV plasmid that was attached to a NOS plate via nucleotide amines. Concentrations of plasmid tested was from 100 fM to 100 nM were tested. Other steps and the detection probe used is as above. FIG. 26 shows detection in the picomolar range of a supercoiled plasmid which was detectable without sample manipulation (e.g. cleavage).

### Example 17 Detection of SARS-CoV-2 DNA on PVDF

The capture oligo probe can also be non-covalently attached.

A SARS-CoV-2 plasmid, IDT CAT10006625, was detected with Capture oligonucleotide probe absorbed to High Binding 0.45 micro PVDF 96 well filter plates. “DNA ELiMSA” performed by Capture DNA immobilized by adsorption (n=3). Capture DNA was added to the PVDF. The PVDF was blocked with 3% BSA for 1 h and washed 3 times with 20 mM Tris pH8.00+1 mM EDTA followed by 3 times with Binding Buffer (20 mM Tris pH8.00+1M NaCl+1 mM EDTA). Target nucleic acid molecule (e.g. Covid plasmid) and detection oligonucleotide probe DNA were heated to 95 C and added to each well of the plate to start DNA hybridization for around 1.5 h, and washed 3 times with Binding Buffer (20 mM Tris pH8.00+1 M NaCl+1 mM EDTA). The plate was blocked with 1% BSA for 5 min and then incubated with APSA solution in 1% BSA for 15 min, and washed 10 times with designated buffers (6× quick wash with Binding Buffer (20 mM Tris pH8.00+1M NaCl+1 mM EDTA), 3×5 min with 20 mM Tris pH8.00+1M NaCl (no EDTA), and 2× with 20 mM Tris pH8.00+2M AMBIC (1×5 min and 1×15 min). The plate was then incubated with 1 mM AMP for 2 h before collecting the assay products.

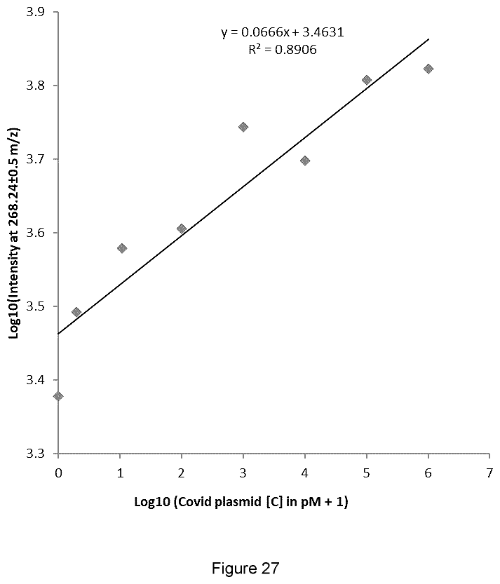

FIG. 27 shows a graph of MS signal intensity at m/z 268 at different concentrations of SARS-CoV-2 target nucleic acid molecule (eg. double stranded coiled plasmid)) (1 μM to 1 μM). The method used herein was able to detect SARS-CoV-2 target nucleic acid molecule at concentrations as low as 1 μM. Linear detection over about 6 logs was possible. In this embodiment the target nucleic acid was a Covid plasmid and the target was adsorbed to PVDF (e.g. non covalent attachment of a double stranded plasmid).

### Example 18

Several methods shown in FIG. 15A using a capture probe and tagged primer are exemplified herein. These can be referred to as half sandwich methods as a detection probe is not used in these assays.

Various single stranded capture oligonucleotide probes were attached to NOS plates via an amine functionality. Other attachments can also be used. Attachment to the solid surface was either via the 3′ or 5′ end of the capture oligonucleotide probe (e.g. the amine functionality could be on the 3′ or the 5′ end or both). Both 3′ and 5′ attachments were tested and both were shown to allow detection. Both antisense and sense strands were attached and both shown to allow detection. If an antisense strand was attached to the solid support, a 5′ biotinylated forward primer was used. If a sense strand was attached to the solid support, a 5′ biotinylated reverse primer was used. Both sense and antisense strands could be attached and in such case 5′biotin labelled forward and reverse primers can be used.

In one example Biotinylated PCR Primer sequences were used with 3′ AMINE capture probe to NOS plates.

Biotinylated HIV_Primer_3_Forward
(SEQ ID NO. 38)
5′biotin-CCAGGCCAGATGAGAGAACC
Biotinylated HIV_Primer_3_Reverse
(SEQ ID No. 39)
5′biotin-TGAAGCTTGCTCGGCTCTTA

In another example biotinylated PCR primer sequences were used with 5′ amine capture to NOS plates.

The capture probe for reactions using HIV Primer 3 can be CCACCTATCCCAGTAGGAGAAATCTATAAAAGATGGATAATCCTGGGAT (SEQ ID NO: 45) which can be tethered via a 5′ amine. As demonstrated 5′ tethering and 3′ tethering can be advantageously used.

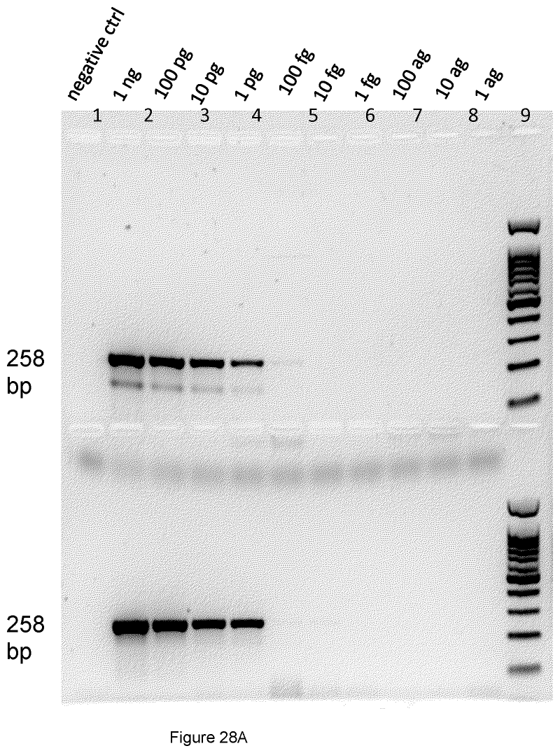

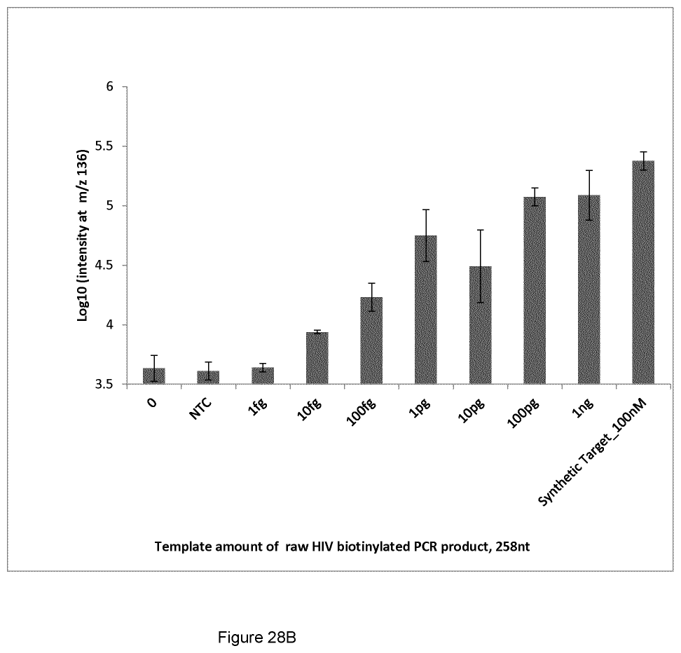

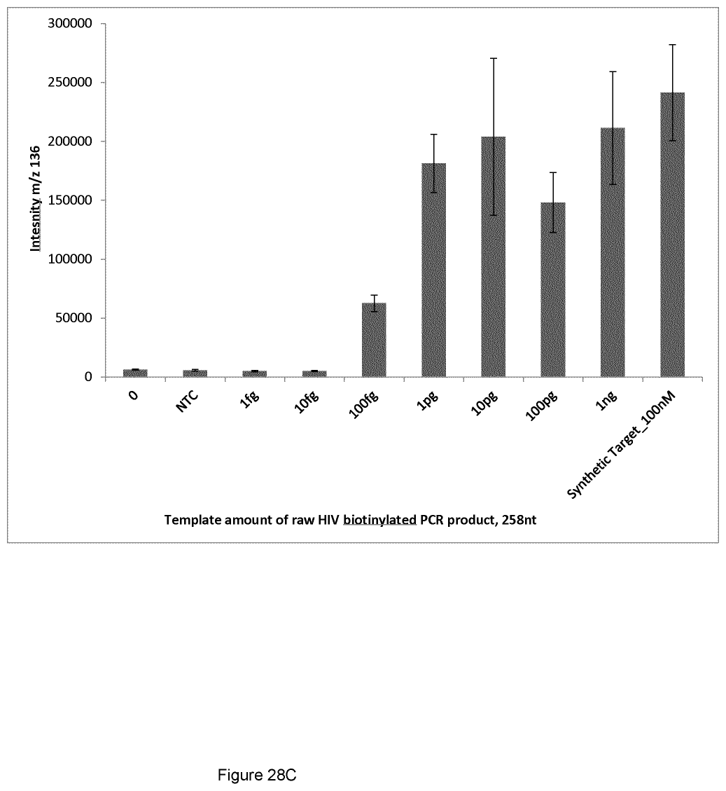

FIGS. 28A and B show results from an assay using a biotinylated forward primer and a biotinylated reverse primer.

FIG. 28A shows PCR reactions from HIV plasmid. 1 ng-1 attogram plasmid was used as template. Reactions initiated with 1 μl HIV plasmid DNA. PCR 35 cycles, lid temp 105 C, 25 uL reaction volume, 94 C melting (30 s), 58 C annealing (30 s), 72 C extension (1 min). The products were separated by a 2% Agarose gel run at 100 volts for 1 hour 20 minutes. 5 ul sample loaded. 3 ul ladder loaded. In Gel staining with GelRed. FIG. 28A Upper gel: BHIV_Primer_3_Forward+HIV_Primer_3_Reverse PCR set. Lower gel HIV_Primer_3 Forward+BHIV_Primer_3 Reverse PCR set.

The amplified antisense strand (capture oligo probe) was 3′ AMINE Captured on a NOS plate. As an antisense strand was covalently attached to the NOS plate, a biotinylated Forward primer is used to prepare the a labelled sense strand that can adhere to the capture oligonucleotide probe. As the PCR strand being amplified is biotinulated via the primer, a detection probe is not per se necessary.

This can be referred to as a half-sandwich assay with NOS_3′-Amine Capture probe. Crude biotin-PCR 258 nt is subjected to mass spectrometry as described herein.

HIV PCR product produced is SEQ ID NO: 40 (258 nt) comprising a Biotin 5′ end eg.: Biotin-5′-SEQ ID NO: 40.

The HIV Capture III probe used in this example is (50 nt): 5′-AATCCCAGGATTATCCATCTTTTATAGATTTCTCCTACTGGGATAGGTGG-3′-Amine (SEQ ID NO: 44).

As mentioned, a Biotin labelled—Forward Primer+unlabelled Reverse Primer were used.

FIG. 28B shows results after detection with mass spectrometry using the method describe below.

The biotin labelled forward primer produced, an HIV biotinylated PCR product, which was 258 nt. The PCR reaction comprising the PCR product was without purification, detected by a half-sandwich HIV PCR DNA ELiMSA (e.g. capture probe, biotinylated primer, reaction with reporter enzyme detection probe and detection of one or more ionizable products) which was performed by Capture DNA immobilized on NOS surface chemistry 96 well polystyrene reactive plates (n=3). 5 uL of the PCR sample solution diluted into 100 uL with Binding Buffer, was used for each DNA ELiMSA reaction. Zero represents no addition of a Target DNA sequence; NTC represents the no-template-control of the PCR reaction; A full-sandwich HIV DNA ELiMSA with a synthetic Target DNA sequence at 100 nM was used as the positive control. Error bar=STDEV

DNA ELiMSA was performed by Capture DNA immobilized on NOS surface chemistry 96 well polystyrene reactive plates. Capture DNA in Surface Binding Buffer (10 mM Na2PO4+1 mM EDTA buffer, pH 8.5) was added to the plate and incubated at 4° C. overnight. Washed 3 times with Binding Buffer (20 mM Tris pH8.00+1 M NaCl+1 mM EDTA), and then quenched and blocked the plate with 3% BSA for 1 h and washed 3 times with 20 mM Tris pH8.00+1 mM EDTA followed by 3 times with Binding Buffer. The PCR products were denatured, as well as the synthetic Target and Detection DNA sequences, and was added to each well of the plate to start DNA hybridization for around 1.5 h, and washed 3 times with Binding Buffer. The plated was blocked with 1% BSA for 5 min and then incubated with APSA solution in 1% BSA for 15 min, and washed 10 times with designated buffers (6× quick wash with Binding Buffer (20 mM Tris pH8.00+1 M NaCl+1 mM EDTA), 3×5 min with 20 mM Tris pH8.00+1M NaCl (no EDTA), and 2× with 20 mM Tris pH8.00+2M AMBIC (1×5 min and 1×15 min). The plate was then incubated with 1 mM AMP for 2 h before collecting the assay products. 1 or 2 μl of the assay products (200 μl) was subjected to mass spectrometry 1 or 2 uL injected (see FIG. 28B). In the gel shown in FIG. 28A, detection of 100 fg target was barely detectable. Using the method described herein, and as shown in FIG. 28B, 100 fg template is clearly detectable. Further as mentioned, only a small fraction (1/100, 1/200) of the assay products are run using mass spectrometry where as the gel uses the full equivalent sample.

A similar assay was performed using 5′ amine attachment of the capture probe and a biotinylated Reverse primer.

In this assay, a single stranded sense strand HIV capture probe was covalently attached to a NOS plate through an amine at its 5′ end.

The HIV Capture III (50 nt) is Amine-5′-CCACCTATCCCAGTAGGAGAAATCTATAAAAGATGGATAATCCTGGGATT-3′ (SEQ ID NO: 45).

In this assay crude PCR product (also referred to as “raw” product) demonstrating the robustness of the method.

HIV PCR product (258 nt): Biotin-5′-TGAAGCTTGCTCGGCTCTTAGAGTTTTATAGAACCGGTCTACATAGTCTCTAAAGGGTTCTTTTGGTCCTT GTCTTATGTCCAGAATGCTGGTAGGGCTATACATTCTTACTATTTTATTTAATCCCAGGATTATCCATCTTT TATAAATTTCTCCTACTGGGATAGGTGGATTATTTGTCATCCATCCTATTTGTTCCTGAAGGGTACTAGTA GTTCCTGCTATGTCACTTCCCCTTGGTTCTCTCATCTGGCCTGG-3 (SEQ ID NO; 46) complementarity to SEQ ID NO: 40

Results are shown in FIG. 28C.

FIG. 28C shows that an HIV biotinylated PCR product, 258 nt, without purification, was detected by a half-sandwich HIV PCR DNA ELiMSA which was performed by Capture DNA immobilized on NOS surface chemistry 96 well polystyrene reactive plates (n=3). 5 uL of the PCR sample solution diluted into 100 uL with Binding Buffer, was used for each DNA ELiMSA reaction. Zero represents no addition of a Target DNA sequence; NTC represents the no-template-control of the PCR reaction; A full-sandwich HIV DNA ELiMSA with a synthetic Target DNA sequence at 100 nM was used as the positive control. Error bar=STDEV

DNA ELiMSA was performed by Capture DNA immobilized on NOS surface chemistry 96 well polystyrene reactive plates. Capture DNA in Surface Binding Buffer (10 mM Na2PO4+1 mM EDTA buffer, pH 8.5) was added to the plate and incubated at 4° C. overnight. Washed 3 times with Binding Buffer (20 mM Tris pH8.00+1 M NaCl+1 mM EDTA), and then quenched and blocked the plate with 3% BSA for 1 h and washed 3 times with 20 mM Tris pH8.00+1 mM EDTA followed by 3 times with Binding Buffer. The PCR products were denatured by heat, as well as the synthetic Target and Detection DNA sequences, and was added to each well of the plate to start DNA hybridization for around 1.5 h, and washed 3 times with Binding Buffer. The plated was blocked with 1% BSA for 5 min and then incubated with APSA solution in 1% BSA for 15 min, and washed 10 times with designated buffers (6× quick wash with Binding Buffer (20 mM Tris pH8.00+1 M NaCl+1 mM EDTA), 3×5 min with 20 mM Tris pH8.00+1M NaCl (no EDTA), and 2× with 20 mM Tris pH8.00+2M AMBIC (1×5 min and 1×15 min). The plate was then incubated with 1 mM AMP for 2 h before collecting the assay products.

In this assay as little as starting template 10 fg produced a reproducible signal.

A further assay used non-covalent attachment and PVDF. In this example, HIV was detected using a half-sandwich assay where the single stranded HIV target sequence was adsorbed onto PVDF and a detection probe that is complementary and labelled with a tag is used to detect the target sequence. No capture probe is used The assay compared 0 vs. 100 pmol target.

HIV Detection III (50 nt): Biotin-5′-CTACATAGTCTCTAAAGGGTTCTTTTGGTCCTTGTCTTATGTCCAGAATG-3′ (SEQ ID NO: 25).

HIV oligo Target III: (133 nt): CCACCTATCCCAGTAGGAGAAATCTATAAAAGATGGATAATCCTGGGATTAAATAAAATAGTAAGAATGTA TAGCCCTACCAGCATTCTGGACATAAGACAAGGACCAAAAGAACCCTTTAGAGACTATGTAG (SEQ ID NO: 24).

HIV half-sandwich DNA ELiMSA was performed by the synthetic Target DNA absorbed to a 0.45 micron PVDF 96 well filter plate (n=3). The PVDF filter plate was pre-wetted by methanol, spotted with Target DNA, blocked with 3% BSA for 1 h, and washed 3 times with 20 mM Tris pH8.00+1 mM EDTA followed by 3 times with Binding Buffer Buffer (20 mM Tris pH8.00+1 M NaCl+1 mM EDTA). Denatured Detection DNA sequence was added to each well of the plate to start DNA hybridization for around 1.5 h, and washed 3 times with Binding Buffer. The plated was blocked with 1% BSA for 5 min and then incubated with APSA solution in 1% BSA for 15 min, and washed 10 times with designated buffers (6× quick wash with Binding Buffer (20 mM Tris pH8.00+1 M NaCl+1 mM EDTA), 3×5 min with 20 mM Tris pH8.00+1 M NaCl (no EDTA), and 2× with 20 mM Tris pH8.00+2M AMBIC (1×5 min and 1×15 min). The plate was then incubated with 1 mM AMP for 2 h before collecting the assay products.

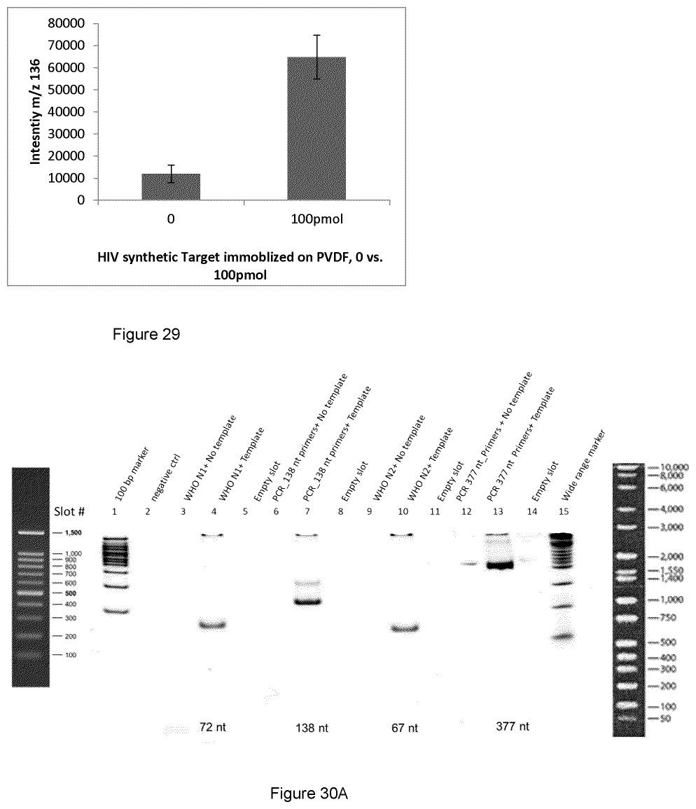

As shown in FIG. 29, the noncovalently attached HIV target sequence could be detected using the “DNA ELIMSA” methods described herein.

### Example 19

Additional assays were conducted adding SDS or non-relevant nucleic acid to assess the robustness of the method.

Covid DNA ELiMSA performed by 5′ N Capture DNA immobilized on NOS surface chemistry 96 well polystyrene reactive plates. Capture DNA in Surface Binding Buffer (10 mM Na2PO4+1 mM EDTA buffer, pH 8.5) was added to the plate and incubated at 4° C. overnight. Washed 3 times with Surface Binding Buffer, and then quenched in 20 mM Tris pH 8.5. The plate was blocked with the 3% BSA and/or blocked with 5 micro grams of salmon sperm DNA either before, during or after or after BSA or during target hybridization for 1 h and washed 3 times with 20 mM Tris pH8.00+1 mM EDTA followed by 3 times with Binding Buffer (20 mM Tris pH8.00+1 M NaCl+1 mM EDTA). The plated was blocked with 1% BSA for 5 min and then incubated with APSA solution in 1% BSA for 15 min, and washed 10 times with designated buffers (6× quick wash with Binding Buffer, 3×5 min with 20 mM Tris pH8.00+1M NaCl (no EDTA), and 2× with 20 mM Tris+2M AMBIC (1×5 min and 1×15 min). The plate was then incubated with 1 mM AMP for 2 h before collecting the assay products. Assay products were measured using mass spectrometry m/z 136.

The addition salmon sperm DNA did not appreciably affect the assay where added before BSA or in combination with BSA or during the hybridization step.

SDS was also added in other tests.

Target and Probe DNA were incubated with 0, 0.1, 0.5, 1 and 2% w/v sodium dodecyl sulfate (SDS) which was added to the designated well of the plate to start DNA hybridization for around 1.5 h. addition of SDS appeared to reduce non-specific binding at long template concentrations. The assay can tolerate high concentrations of SDS or non ionic surfactants.

### Example 20

PCR primers were designed for COVID which worked very well in PCR. They can also be used in methods described herein.

SARS-Co-V 2 PCR reactions were prepared using SARS-Co-V2 plasmid comprising nucleocapsid using the following PCR conditions. The PCR reactions initiated with 10 ng of SARS-CoV-2 positive ctrl Plasmid. (35 cycles, lid temp 105° C., 50 μL reaction volume, 94° C. melting (30 s), 58° C. annealing (30 s), 72° C. extension (1 min)).

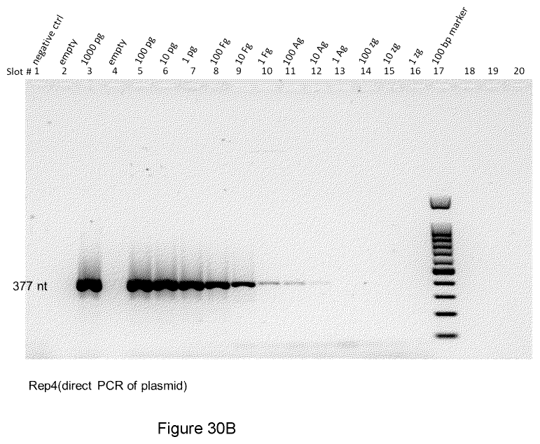

As shown in FIG. 30 10 μl of PCR reactions were loaded for WHO primers (WHO N1 (72 nt) and WHO N2 (67 nt product) and 5 μl PCR samples loaded for primers tested (see Example 11, SEQ ID NOS: 2 ad 3 produced a 138 nt product, and SEQ ID NOS: 7 and 8 produced 377 nt product. 4 μl ladder loaded. Post staining with GelRed for 30 minutes. No bands were present in the absence of template or the absence of DNA template to be amplified. Fragments of the expected size were obtained ranging from 67 to 377 nt depending on the primer pair used. Products produced by primer pairs SEQ ID Nos: 2 and 3 and 7 and 8 produced a stronger band that either pair of WHO primers. See FIG. 30.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

### CITATIONS FOR DOCUMENTS REFERRED TO HEREIN

Banks, J. F., Jr., et al., Ultrasonically assisted electrospray ionization for LC/MS determination of nucleosides from a transfer RNA digest. Anal Chem, 1994. 66(3): p. 406-14.
Basiri A, Heidari A, Nadi M F, Fallahy M T P, Nezamabadi S S, Sedighi M, Saghazadeh A, Rezaei N. Microfluidic devices for detection of RNA viruses. Rev Med Virol. 2020 Aug. 26:e2154.
Bowden, P., et al., Quantitative statistical analysis of standard and human blood proteins from liquid chromatography, electrospray ionization, and tandem mass spectrometry. Journal of proteome research, 2012. 11: p. 2032-2047.
Chen, D. Y. and N. J. Dovichi, Yoctomole detection limit by laser-induced fluorescence in capillary electrophoresis. J Chromatogr B Biomed Appl, 1994. 657(2): p. 265-9.
Chin, C. S., et al., Nonhybrid, finished microbial genome assemblies from long-read SMRT sequencing data. Nat Methods, 2013. 10(6): p. 563-9.
Consortium, E. P., A user's guide to the encyclopedia of DNA elements (ENCODE). PLoS Biol, 2011. 9(4): p. e1001046.
Doddapaneni H, Cregeen S J, Sucgang R, Meng Q, Qin X, Avadhanula V, Chao H, Menon V, Nicholson E, Henke D, Piedra F A, Rajan A, Momin Z, Kottapalli K, Hoffman K L, Sedlazeck F J, Metcalf G, Piedra P A, Muzny D M, Petrosino J F, Gibbs R A. Oligonucleotide capture sequencing of the SARS-CoV-2 genome and subgenomic fragments from COVID-19 individuals. bioRxiv [Preprint]. 2020 Jul. 27:2020.07.27.223495. doi: 10.1101/2020.07.27.223495.
Engvall, E. and P. Perlman, Enzyme-linked immunosorbent assay (ELISA). Quantitative assay of immunoglobulin G. Immunochemistry, 1971. 8(9): p. 871-4.
Florentinus, A. K., et al., The Fc receptor-cytoskeleton complex from human neutrophils. Journal of proteomics, 2011. 75: p. 450-468.
Florentinus-Mefailoski, A. and J. G. Marshall, A pyridoxamine-5-phosphate ELIMSA substrate for linear absolute quantification of alkaline phosphatase to the yoctomole range applied to prostate specific antigen. Anal Chem, 2014.
Florentinus-Mefailoski, A. and J. G. Marshall, Linear quantification of a streptavidin-alkaline phosphatase probe for enzyme-linked immuno mass spectrometric assay. Anal Biochem, 2016. 503: p. 50-5.
Florentinus-Mefailoski, A., et al., An enzyme-linked immuno-mass spectrometric assay with the substrate adenosine monophosphate. Anal Bioanal Chem, 2015. 407(4): p. 1119-30.
Florentinus-Mefailoski, A., F. Safi, and J. G. Marshall, Enzyme Linked Immuno Mass Spectrometric Assay (ELIMSA). J Proteomics, 2014. 96: p. 343-52.
Fritz, E., et al., Quantification of Coxiella burnetii by polymerase chain reaction (PCR) and a colorimetric microtiter plate hybridization assay (CMHA). Eur J Epidemiol, 1995. 11(5): p. 549-57.
Jiao J, Duan C, Xue L, Liu Y, Sun W, Xiang Y. DNA nanoscaffold-based SARS-CoV-2 detection for COVID-19 diagnosis. Biosens Bioelectron. 2020 Nov. 1; 167:112479. doi: 10.1016/j.bios.2020.112479.
Li, H., et al., Peptide and Protein Quantification Using Automated Immuno-MALDI (iMALDI). J Vis Exp, 2017(126).
Mansfield, M. A., Rapid immunodetection on polyvinylidene fluoride membrane blots without blocking. Anal Biochem, 1995. 229(1): p. 140-3.
Marshall, J., et al., Involvement of cytosolic phospholipase A2 and secretory phospholipase A2 in arachidonic acid release from human neutrophils. J Immunol, 2000. 164(4): p. 2084-91.
Munge, B., et al., Multiple enzyme layers on carbon nanotubes for electrochemical detection down to 80 DNA copies. Anal Chem, 2005. 77(14): p. 4662-6.
Onisko, B., et al., Mass spectrometric detection of attomole amounts of the prion protein by nanoLC/MS/MS. J Am Soc Mass Spectrom, 2007. 18(6): p. 1070-9.
Razumienko, E., et al., Element-tagged immunoassay with ICP-MS detection: evaluation and comparison to conventional immunoassays. J Immunol Methods, 2008. 336(1): p. 56-63.
Rissin, D. M., et al., Single-molecule enzyme-linked immunosorbent assay detects serum proteins at subfemtomolar concentrations. Nat Biotechnol, 2010. 28(6): p. 595-9.
Ronaghi, M., et al., Real-time DNA sequencing using detection of pyrophosphate release. Anal Biochem, 1996. 242(1): p. 84-9.
Rutledge, R. G. and C. Cote, Mathematics of quantitative kinetic PCR and the application of standard curves. Nucleic Acids Res, 2003. 31(16): p. e93.
Saiki, R. K., et al., Enzymatic amplification of beta-globin genomic sequences and restriction site analysis for diagnosis of sickle cell anemia. Science, 1985. 230(4732): p. 1350-4.
Santhanam M, Algov I, Alfonta L. DNA/RNA Electrochemical Biosensing Devices a Future Replacement of PCR Methods for a Fast Epidemic Containment. Sensors (Basel). 2020 Aug. 18; 20(16):4648.
Shukla, R., et al., Quantitative determination of human interleukin 22 (IL-22) in serum using Singulex-Erenna® technology. J Immunol Methods, 2013. 390(1-2): p. 30-4.
Sun, X., N. Gao, and W. Jin, Monitoring yoctomole alkaline phosphatase by capillary electrophoresis with on-capillary catalysis-electrochemical detection. Anal Chim Acta, 2006. 571(1): p. 30-3.
Tangemann, K. and J. Engel, Demonstration of non-linear detection in ELISA resulting in up to 1000-fold too high affinities of fibrinogen binding to integrin alpha IIb beta 3. FEBS Lett, 1995. 358(2): p. 179-81.
To, K. K., et al., Temporal profiles of viral load in posterior oropharyngeal saliva samples and serum antibody responses during infection by SARS-CoV-2: an observational cohort study. Lancet Infect Dis, 2020.
Tobos, C. I., et al., Sensitivity and binding kinetics of an ultra-sensitive chemiluminescent enzyme-linked immunosorbent assay at arrays of antibodies. J Immunol Methods, 2019. 474: p. 112643.
Tucholska, M., et al., Human serum proteins fractionated by preparative partition chromatography prior to LC-ESI-MS/MS. Journal of proteome research, 2009. 8: p. 1143-1155.
Van Weemen, B. K. and A. H. Schuurs, Immunoassay using antigen-enzyme conjugates. FEBS Lett, 1971. 15(3): p. 232-236.
Vandamme, A. M., et al., Detection of HIV-1 RNA in plasma and serum samples using the NASBA amplification system compared to RNA-PCR. J Virol Methods, 1995. 52(1-2): p. 121-32.
Vermisoglou E, Panáček D, Jayaramulu K, Pykal M, Frébort I, Kolář M, Hajdúch M, Zbořil R, Otyepka M. Human virus detection with graphene-based materials. Biosens Bioelectron. 2020 Oct. 15; 166:112436.
Walt, D. R., Optical methods for single molecule detection and analysis. Anal Chem, 2013. 85(3): p. 1258-63.
Yalow, R. S. and S. A. Berson, Immunoassay of endogenous plasma insulin in man. J Clin Invest, 1960. 39: p. 1157-75.
Xie, X., et al., Chest C T for Typical 2019-nCoV Pneumonia: Relationship to Negative RT-PCR Testing. Radiology, 2020: p. 200343.
Xiao, A. T., Y. X. Tong, and S. Zhang, False negative of RT-PCR and prolonged nucleic acid conversion in COVID-19: Rather than recurrence. J Med Virol, 2020.
Xu, Y. and Zheng Z., Direct RNA detection without nucleic acid purification and PCR: Combining sandwich hybridization with signal amplification based on branched hybridization chain reaction. Biosens Bioelectron. 2016 May 15; 79:593-9. doi: 10.1016/j.bios.2015.12.057. Epub 2015 Dec. 21.

1. A method of detecting a target nucleic acid molecule comprising
a.
i. incubating a sample putatively comprising the target nucleic acid molecule with a capture oligonucleotide probe that comprises a sequence complementary to the target nucleic acid molecule and that is attached to a solid phase, in a first binding solution, optionally wherein the solid phase is attached to the capture oligonucleotide probe through a linker; or
ii. incubating a sample putatively comprising the target nucleic acid molecule with a solid phase to attach said sample/target nucleic acid molecule to said solid phase, in a first binding solution, optionally wherein the solid phase is attached to the sample/target nucleic acid molecule through a linker;
b. binding any target nucleic acid molecule to a detection oligonucleotide probe in a second binding solution under conditions for forming a target:detection complex;
c. incubating any target:detection complex with a reporter enzyme detection probe in a third binding solution under conditions for forming a target:detection:enzyme complex;
d. washing the solid phase to remove any unbound reporter enzyme detection probe with a washing solution;
e. incubating any target:detection:enzyme complex with a reporter enzyme detection probe substrate in a substrate reaction solution to generate one or more ionizable products; and
f. detecting at least one of the one or more ionizable products using mass spectrometry (MS),
wherein
at least the second binding solution and the third binding solution among the first binding solution, the second binding solution, and the third binding solution are substantially free of inorganic salt; and
the washing solution is substantially free of inorganic salt;
wherein the second binding solution, the third binding solution, and the washing solution each independently is a volatile solution comprising a volatile buffer selected from ethanolamine, ammonium bicarbonate, ammonium formate, pyridinium formate, trialkylammonium/formic acid, ammonium acetate, trialkylammonium bicarbonate, N-ethylmorpholine/acetate, trialkylammonium acetate, or combinations thereof, and
wherein optionally the method further comprises cross-linking components of any target:detection:enzyme complex and the capture oligonucleotide probe prior to the step d) and the step e);
wherein optionally the method further comprises separating the one or more ionizable products prior to detection using MS; and
wherein detection of the at least one of the one or more ionizable products is indicative of the sample comprising the target nucleic acid molecule.

2. The method of claim 1, wherein the second binding solution, the third binding solution and the substrate reaction solution each comprises a Tris buffer.

3. The method of claim 1 or 2, wherein the capture oligonucleotide probe is directly immobilized to the solid phase, optionally by non-covalent or covalent binding to the solid phase or the detection oligonucleotide probe is a detection oligonucleotide primer and the step comprises amplifying the target nucleic acid molecule with a detection oligonucleotide primer, in an amplification solution and binding any amplified target to the detection oligonucleotide probe in the second binding solution under conditions for forming a target:detection complex.

4. The method of any one of claims 1 to 3, wherein the method comprises separating the one or more ionizable products prior to detection using MS.

5. The method of any one of claims 1 to 4, wherein the separation is by liquid chromatography.

6. The method of claim 5, wherein the liquid chromatography is selected from normal phase chromatography, reverse phase chromatography, and high-performance liquid chromatography (HPLC).

7. The method of claim 6, wherein the liquid chromatography is isocratic.

8. The method of any one of claims 1 to 7, wherein the step of detecting the one or more ionizable products using MS comprises ionizing the one or more ionizable products, optionally by electrospray ionization (ESI), MALDI, chemical ionization, electron impact, laser desorption, electrical ionization, or heat ionization to produce one or more product ions, and subjecting the one or more product ions to MS optionally tandem MS (MS/MS).

9. The method of claim 8, wherein the ionizing is positive ionization or negative ionization.

10. The method of claim 8 or 9, wherein the produced one or more product ions have a selected signal to noise ratio that is at least 3, at least 4, at least 5, at least 6, at least 7, at least 8, at least 9 or at least 10.

11. The method of any one of claims 1 to 10, wherein the MS is selected from electrospray ionization tandem MS (ESI-MS/MS), matrix-assisted laser desorption/ionization time-of-flight (MALDI-TOF), tandem MS (MS/MS), multiple rounds of fragmentation MSN, MALDI, electrospray, nanospray, surface ionization, laser desorption & ionization, atmospheric ionization, vacuum ionization, and MS equipped with capillary electrophoresis, ultra sonic or sonic or vibration, nanodroplet or microdroplet sample introduction system.

12. The method of any one of claims 1 to 11, wherein the detecting using MS comprises recording product ion intensity by single ion monitoring (SIM) and/or product ion parent to fragment transition by single reagent monitoring (SRM).

13. The method of any one of claims 1 to 12, wherein the capture oligonucleotide probe comprises a oligonucleotide that has a sequence complementary to a part the target nucleic acid molecule that is at least 25 nucleotides in length, at least 35 nucleotides in length, optionally the capture oligonucleotide probe has a sequence complementary to a part of the sequence of the target nucleic acid molecule that is about 30 nucleotides to about 60 nucleotides in length, or about 40 nucleotides to about 55 nucleotides in length.

14. The method of claim 13, wherein the detection oligonucleotide probe comprises an oligonucleotide that has a sequence complementary to another part of the target nucleic acid molecule, and a secondary target moiety selected from biotin, ALFA-tag, AviTag, C-tag, Calmoudulin-Tag, Polyglutamate Tag, E-Tag, Flag-tag, HA-tag, His-Tag, myc-Tag, NE-tag, Rho1D4-Tag, S-Tag, SBP-Tag, Softag 1, Softag 3, Spot-tag, Strept-tag, T7-tag, TC-tag, Ty1 tag, V5 tag, VSV-tag, Xpress tag, Isopeptag, SpyTag, SnoopTag, DogTag, Sdy Tag, Biotin carboxyl carrier protein, glutathione-S-transferase tas, GFP tag, HaloTag, SNAP-tag, CLIP-tag, HUH-Tag, Maltose-binding protein tag, Nus-tag, thioredoxin-tag, Fc-tag, and CRDSAT-tag, optionally the second target moiety is biotin.

15. The method of claim 14, wherein the sequence of the oligonucleotide of the detection oligonucleotide probe complementary to the other part of the target nucleic acid molecule is at least 25 nucleotides in length, at least 35 nucleotides in length, optionally the detection oligonucleotide probe is about 30 nucleotides to about 60 nucleotides in length, or about 40 nucleotides to about 55 nucleotides in length.

16. The method of any one of claims 1 to 15, wherein the capture oligonucleotide probe and the detection oligonucleotide probe can both bind the target nucleic acid molecule at non-overlapping regions, optionally the non-overlapping regions are adjacent, optionally the non-overlapping regions are at least one nucleotide apart, optionally the non-overlapping regions are at least 5 nucleotides apart, optionally the non-overlapping regions are about 2 nucleotides, about 5 nucleotides, about 10 nucleotides, about 20 nucleotides, or about 25 nucleotides apart.

17. The method of any one of claims 1 to 16, wherein the reporter enzyme detection probe comprises a reporter enzyme and optionally a secondary target binding moiety, and wherein the secondary target binding moiety is covalently bound to the reporter enzyme.

18. The method of claim 17, wherein the secondary target binding moiety binds the secondary target moiety of the detection oligonucleotide probe and is selected from avidin, streptavidin, calmodulin, anion-exchange resin, Mono-Q, cation-exchange resin, anti-E-tag antibody, anti-FLAG-tag antibody, anti-HA-tag antibody, nickel or cobalt chelate, anti-Myc-tag antibody, anti-NE-tag antibody, anti-Rho1D4-tag antibody, anti-S-tag antibody; anti-Softag 1 antibody, anti-Softag 3 antibody, nanobody, streptactin, anti-T7-tag antibody, FlAsH biarsenical compounds, ReAsH biarsenical compounds, anti-Ty1 tag antibody, anti-V5 tag antibody, anti-VSV tag antibody, anti-Xpress tag antibody, pilin-C protein, SpyCatcher protein, SnoopCatcher protein, SnoopTagJr protein, SdyCatcher protein, glutathione, GFP-antibody, haloalkane substrate, benzylguanine derivatives, benzylcytosine derivatives, HUH specific DNA sequence, amylose agarose, Nus-tag antibody, anti-thioredoxin-tag antibody, protein-A sepharose, lactose, agarose, optionally the secondary target binding moiety is selected from avidin, and streptavidin when the secondary target moiety is biotin.

19. The method of claim 17 or 18, wherein the reporter enzyme is selected from a phosphatase, optionally alkaline phosphatase, lyase, hydrolase, synthase, synthetase, oxidoreductase, dehydrogenase, oxidase, transferease, isomerase, ligase, protease, such as trypsin, proteinase, peroxidase, glucose oxidase, myeloperoxidase, oxidase, monooxygenase, cytochrome, decarboxylase, lipase, caspase, amylase, peptidase, transaminase, kinase, DNA or RNA polymerase, optionally TAQ, restriction enzyme, klenow fragment, and DNA ligase.

20. The method of claim 19, wherein the reporter enzyme is selected from alkaline phosphatase, horseradish peroxidase, trypsin, cytochrome C monooxygenase, and myeloperoxidase, optionally, the reporter enzyme is alkaline phosphatase or horseradish peroxidase.

21. The method of any one of claims 1 to 20, wherein the one or more ionizable products are readily ionizable under ESI-MS/MS or MALDI-TOF and generates a product ion characterized by a high signal to noise ratio, and the substrate is optionally selected from:
a. a phosphorylated nucleoside, optionally AMP or CMP, or nucleotide, optionally ATP or CTP, phosphorylated alkaloid, phosphorylated amino acid, phosphorylated amino acid polymer, and phosphorylated metabolite when the enzyme is alkaline phosphatase (AP);
b. a compound selected from phenols, amines, optionally phenolic amines, aromatic compounds, olefin halogenations, luminol, pyrogallol, 2,2′-azino-bis(3-ethylbenzothiazoline-6-sulphonic acid (ABTS), and Amplex® Red when the reporter enzyme is horseradish peroxidase (HRP); or from
c. opiates, detergents, dye precursor, alcohols, and matrix.

22. The method of claim 21, wherein the reporter enzyme detection probe substrate is selected from pyridoxamine-5-phosphate (PA5P), p-nitrophenyl phosphate (PNPP), Amplex® Red (AR), naphthol ASMX phosphate, luminol, Lumigen® TMA3, Lumigen® TMA6, sphingosine, 4MUP, L-(+)-2-amino-6-phosphonohexanoic acid, 5-bromo-4-chloro-3-indolyl phosphate (BCIP), BluePhos®, phenylbenzene ω phosphono-α-amino acid, O-phospho-DL-threonine, adenosine monophosphate (AMP), AR (3-amino-9-ethylcarbazole), 4-CN (4-chloro-1-naphtol), DAB (3,3′-DiAminoBenzimidine), OPD (o-phenylene diamine), TMB (3,3″,5,5″-tetramethylbenzidine), pNPP (p-nitrophenyl phosphate), NBT (nitroblue tetrazolium), INT (p-iodonitrotetrazolium), MUP (4-methylumbelliferyl phosphate), and FDP fluorescein diphosphate), pyrogallol.

23. The method of claim 22, wherein the reporter enzyme detection probe substrate is selected from:
a. AR, luminol, Lumigen® TMA3, and Lumigen® TMA6, when the reporter enzyme detection probe comprises HRP; or from
b. naphthol ASMX phosphate, and PNPP, when the reporter enzyme detection probe comprises AP.

24. The method of any one of claims 1 to 23, wherein the first binding solution is a second volatile solution comprising a volatile buffer selected from ethanolamine, ammonium bicarbonate, ammonium formate, pyridinium formate, trialkylammonium/formic acid, ammonium acetate, trialkylammonium bicarbonate, N-ethylmorpholine/acetate, trialkylammonium acetate, or combinations thereof.

25. The method of any one of claims 1 to 24, wherein the volatile buffer is selected from ammonium bicarbonate, ethanolamine, ammonium acetate, trialkylammonium bicarbonate, or combinations thereof.

26. The method of claim 25, wherein the trialkylammonium is selected from trimethylammonium, triethylammonium, or combinations thereof.

27. The method of any one of claims 1 to 26, wherein the volatile buffer is ammonium bicarbonate.

28. The method of any one of claims 1 to 27, wherein when the first binding solution, the second solution, the third binding solution, and/or the washing solution is substantially free of inorganic salt, the first binding solution, the second solution, the third binding solution, and/or the washing solution each independently comprises ammonium bicarbonate, optionally the second binding solution and the third binding solution each comprises ammonium bicarbonate, optionally the first binding solution, the second binding solution, and the third binding solution each comprises ammonium bicarbonate, optionally the washing solution comprises ammonium bicarbonate.

29. The method of any one of claims 1 to 28, wherein step a) and step b) are performed simultaneously, and the first binding solution of step a) is the second binding solution of step b).

30. The method of any one of claims 1 to 29 further comprising washing the solid phase with the second binding solution prior to incubating the target:detection complex with the reporter enzyme detection probe.

31. The method of any one of claims 1 to 30 further comprising washing the solid phase with a blocking agent, optionally bovine serum albumin (BSA), prior to incubating the target nucleic acid molecule with the capture oligonucleotide probe.

32. The method of any one of claims 1 to 31, wherein the first binding solution, the second binding solution, the third binding solution, and the substrate reaction solution each independently has a pH of about 7 to about 10, optionally of about 7 to about 8, optionally about 8.8.

33. The method of any one of claims 1 to 32, wherein the substrate reaction solution comprises a non-ionic non polymeric detergent, optionally selected from N-octylglucoside, deoxycholate, rapigest, octyl-beta-glucopyranoside, octylglucopyranoside, chaps, big chap, non-ionic acid labile surfactants, glucosides, n-Octyl-β-D-glucopyranoside, n-Nonyl-(3-D-glucopyranoside thioglucosides, n-Octyl-β-D-thioglucopyranoside maltosides, n-Decyl-β-D-maltopyranoside, n-Dodecyl-β-D-maltopyranoside, n-Undecyl-β-D-maltopyranoside, n-Tridecyl-β-D-maltopyranoside, cymal-5, cymal-6, thiomaltosides, n-Dodecyl-β-D-thiomaltopyranoside, alkyl glycosides, octyl glucose neopentyl glycol, polyoxyethylene glycols, triton, NP40, Tween™, Tween™ 20, Triton X-100, triton x-45, C8E4, C8E5, C10E5, C12E8, C12E9, Brij, Anapoe-58, Brij-58, and combinations thereof.

34. The method of any one of claims 23 to 33, wherein the substrate reaction solution further comprises 4-iodophenylboronic acid when the substrate comprises luminol.

35. The method of any one of claims 1 to 34, wherein the solid phase is a reaction vessel optionally a bead, a plate, a capillary, a filter, or a nano/micro/milli well reaction vessel, and wherein the surface is selected from paper, nitrocellulose, acrylate, plastic, polystyrene, polyvinylene fluoride (PVDF), melamine, silica, polylysine coated glass, 3-aminopropyl-triethoxysilane (APTES) treated glass, and 3-aminopropyl-trimethoxysilane (APTMS) treated glass.

36. The method of any one of claims 1 to 35, wherein the attaching of the capture oligonucleotide probe to the solid phase is through H-hydroxysuccinimide (NHS), N-oxysuccinimide (NOS), maleimide, hydrazide, glutaraldehyde coupling, or PEG crosslinking.

37. The method of any one of claims 1 to 36, wherein the target:detection:enzyme complex is incubated with the reporter enzyme detection probe substrate in the substrate reaction solution to generate the one or more ionizable products for a period of time less than 72 hours, less than 24 hours, less than 12 hours, less than 60 minutes, less than 50 minutes, less than 40 minutes, less than 30 minutes, less than 20 minutes, less than 15 min, less than 10 min, less than 5 min, less than 2 min, or less than 1 min.

38. The method of any one of claims 12 to 37, wherein the product ion is assayed by SIM and/or SRM using an optimized fragmentation energy and m/z range.

39. The method of claim 21, wherein the substrate is AMP, ADP or ATP and one or the ionizable products generated comprises adenosine, the product ion of which is assayed by SIM at 268 m/z; or the substrate is CMP, CDP or CTP and one or the ionizable products generated comprises cytosine, the product ion of which is assayed by SIM at 283 m/z; or the substrate is AR and one of the one or more ionizable products generated comprises resorufin, the product ion of which is assayed by SIM at 214 m/z and SRM using the major intense fragment at 214-186 m/z.

40. The method of claim 22, wherein the substrate is naphthol ASMX phosphate and one of the one or more ionizable products generated comprises dephosphorylated naphthol ASMX, the product ion of which is assayed by SIM at 292 m/z and SRM using the major intense fragment at 292-171 m/z or the substrate is PA5P and one or the ionizable products generated comprises PA, the product ion of which is assayed by SIM at 169 m/z.

41. The method of any one of claims 1 to 40, wherein the ionizable products are ionized to product ions in ionization solution.

42. The method of any one of claims 1 to 41, wherein at least the third binding solution among the first binding solution, the second binding solution, and the third binding solution is substantially free of inorganic salt and comprises a volatile buffer as defined in any one of claims 24 to 27.

43. The method of any one of claims 1 to 41, wherein the method comprises washing the solid phase to remove any unbound reporter enzyme detection probe with the washing solution, wherein the washing solution is substantially free of inorganic salt and comprises a volatile buffer as defined in any one of claims 24 to 27.

44. The method of any one of claims 1 to 41, wherein the components of any target:detection:enzyme complex and the capture oligonucleotide probe are cross-linked prior to the optional step d) and the step e), and the cross-linking is through H-hydroxysuccinimide (NHS), N-oxysuccinimide (NOS), maleimide, hydrazide, glutaraldehyde coupling, disuccinimidyl suberate (DSS) cross-linking or PEG crosslinking.

45. The method of claim 43, wherein the cross-linking of the components of any target:detection:enzyme complex and the capture oligonucleotide probe is through glutaraldehyde coupling, DSS cross-linking, or PEG cross-linking.

46. A method of quantifying the amount of a target nucleic acid molecule in a sample comprising the steps;
a. detecting a target nucleic acid molecule according to the method of any one of claims 1-45; and
b. quantifying the amount of target nucleic acid molecule in the sample based on the intensity of the signal for one or more of the ionizable products detected by mass spectrometry.

47. The method of claim 46, wherein the quantification comprises comparing the intensity of the signal for one or more products against signal intensities generated using known quantities of target substance, under similar conditions.

48. The method of claim 46 or 47, wherein the target nucleic acid molecule is present or suspected to be present in the sample in or up to a pico mol, femto mol, or atto mol range.

49. The method of any one of claims 1 to 48, the target nucleic acid molecule is selected from DNA, RNA, and combinations and derivatives thereof.

50. The method of any one of claims 46 to 49, wherein the sample is a biological sample, industrial product, environmental sample, or a polymerase chain reaction (PCR) reaction product.

51. The method of claim 50, wherein the biological sample is a blood sample, urine sample, fecal sample, effusate, tissue sample or sputum sample.

52. A method of detecting a target nucleic acid molecule comprising
performing a nucleic acid amplification such as a polymerase chain reaction (PCR) or a hybridization chain reaction (HCR) or rolling circle reaction or other nucleic acid reaction on a test sample putatively comprising the target nucleic acid molecule with a modified primer and a second primer to obtain an amplified nucleic acid product, optionally a PCR product, comprising the modified primer, the modified primer being functionalized with a secondary target moiety or a reporter enzyme;
separating the amplified nucleic acid product from any unreacted modified primer;
when the modified primer is functionalized with the secondary target moiety, incubating the amplified nucleic acid product with a reporter enzyme detection probe in a first binding solution under conditions to form an amplified nucleic acid product:reporter enzyme complex, and removing any unbound reporter enzyme detection probe with a washing solution, the reporter enzyme detection probe comprising a secondary target binding moiety and a reporter enzyme;
incubating the amplified nucleic acid product or the amplified nucleic acid product:reporter enzyme complex with a reporter enzyme substrate in a substrate reaction solution to generate one or more ionizable products; and
detecting the one or more ionizable products using mass spectrometry (MS),
wherein when the modified primer is a forward primer, the second primer is a reverse primer, and
wherein when the modified primer is a reverse primer, the second primer is a forward primer.

53. The method of claim 52, wherein the second primer is attached to a solid phase, optionally the second primer is attached to the solid phase through a linker.

54. The method of claim 53, wherein the second primer is directly attached to the solid phase, optionally by non-covalent or covalent binding to the solid phase.

55. The method of claim 53 or 54, wherein the separation of the unreacted modified primer from the amplified nucleic acid product is by centrifugation, filtration and/or solvent wash.

56. The method of claim 52, wherein the method further comprises incubating the amplified nucleic acid product comprising the modified primer with a solid phase in a second binding solution under conditions to bind the amplified nucleic acid product onto the solid phase, prior to incubating the amplified nucleic acid product with the reporter enzyme detection probe, the solid phase having a capture oligonucleotide probe attached thereon that comprises a sequence complementary to the amplified nucleic acid product, optionally, the solid phase is attached to the capture oligonucleotide probe through a linker.

57. The method of claim 56, wherein the capture oligonucleotide probe is directly attached to the solid phase, optionally by non-covalent or covalent binding to the solid phase.

58. The method of any one of claims 52 to 57, wherein the first binding solution and/or the washing solution is volatile and substantially free of NaCl.

59. The method of any one of claims 56 to 57, wherein the second binding solution being volatile and substantially free of NaCl.

60. The method of claim 58 or 59, wherein the first binding solution or the second binding solution each comprises a volatile buffer.

61. The method of claim 60, wherein the volatile buffer is selected from ethanolamine, ammonium bicarbonate, ammonium formate, pyridinium formate, trialkylammonium/formic acid, ammonium acetate, trialkylammonium bicarbonate, N-ethylmorpholine/acetate, trialkylammonium acetate, and combinations thereof.

62. The method of claim 61, wherein the volatile buffer is selected from ethanolamine, ammonium acetate, trialkylammonium bicarbonate, and combinations thereof.

63. The method of claim 60 or 61, wherein the trialkylammonium is selected from trimethylammonium, triethylammonium, and combinations thereof.

64. The method of claim 62 or 63, wherein the volatile buffer is ethanolamine.

65. The method of claim 56 further comprising washing the solid phase with a blocking agent, optionally bovine serum albumin (BSA), prior to binding the amplified nucleic acid product to the solid phase.

66. The method of claim 60, wherein the first binding solution or the second binding solution each independently has a pH of about 7 to about 10, optionally of about 7 to about 8, optionally about 8.8.

67. The method of any one of claims 52 to 66, wherein the removing of any unbound reporter enzyme detection probe from the amplified nucleic acid product:reporter enzyme complex is by centrifugation, filtration and/or solvent wash.

68. The method of any one of claims 52 to 67 further comprising separating the one or more ionizable products prior to detection using MS.

69. The method of claim 68, wherein the separation is by liquid chromatography, optionally normal phase chromatography or reverse phase chromatography, optionally the chromatography is isocratic.

70. The method of claim 69, wherein the liquid chromatography is size exclusion chromatography, gel permeation chromatography, ion exchange chromatography, normal phase chromatography, reverse phase chromatography, affinity chromatography, electrophoretic separation, capillary electrophoresis, high-performance liquid chromatography (HPLC), and combinations thereof.

71. The method of claim 70, wherein the HPLC is nanoflow liquid chromatography.

72. The method of any one of claims 52 to 71, wherein the step of detecting the one or more ionizable products using MS comprises ionizing the one or more ionizable products, optionally by electrospray ionization (ESI), MALDI, chemical ionization, electron impact, laser desorption, electrical ionization, or heat ionization to produce one or more product ions with a selected signal to noise ratio, and subjecting the one or more product ions to MS optionally tandem MS (MS/MS).

73. The method of claim 72, wherein the ionizing is positive ionization or negative ionization.

74. The method of claim 73, wherein the selected signal to noise ratio is at least 3, at least 4, at least 5, at least 6, or at least 10.

75. The method of any one of claims 52 to 74, wherein the MS is selected from electrospray ionization tandem MS (ESI-MS/MS), matrix-assisted laser desorption/ionization time-of-flight (MALDI-TOF), tandem MS (MS/MS), multiple rounds of fragmentation MSN, MALDI, electrospray, nanospray, surface ionization, laser desorption & ionization, atmospheric ionization, vacuum ionization, and MS equipped with capillary electrophoresis, ultra sonic or sonic or vibration, nanodroplet or mivrodroplet sample introduction system.

76. The method of any one of claims 52 to 75, wherein detection using MS comprises recording product ion intensity by single ion monitoring (SIM) and/or product ion parent to fragment transition by single reagent monitoring (SRM).

77. The method of any one of claims 52 to 76, wherein the secondary target moiety is selected from biotin, ALFA-tag, AviTag, C-tag, Calmoudulin-Tag, Polyglutamate Tag, E-Tag, Flag-tag, HA-tag, His-Tag, myc-Tag, NE-tag, Rho1D4-Tag, S-Tag, SBP-Tag, Softag 1, Softag 3, Spot-tag, Strept-tag, T7-tag, TC-tag, Ty1 tag, V5 tag, VSV-tag, Xpress tag, Isopeptag, SpyTag, SnoopTag, DogTag, Sdy Tag, Biotin carboxyl carrier protein, glutathione-S-transferase tas, GFP tag, HaloTag, SNAP-tag, CLIP-tag, HUH-Tag, Maltose-binding protein tag, Nus-tag, thioredoxin-tag, Fc-tag, and CRDSAT-tag, optionally the second target moiety is biotin.

78. The method of any one of claims 52 to 77, wherein the secondary target binding moiety binds the secondary target moiety and is selected from avidin, streptavidin, calmodulin, anion-exchange resin, Mono-Q, cation-exchange resin, anti-E-tag antibody, anti-FLAG-tag antibody, anti-HA-tag antibody, nickel or cobalt chelate, anti-Myc-tag antibody, anti-NE-tag antibody, anti-Rho1D4-tag antibody, anti-S-tag antibody, anti-Softag 1 antibody, anti-Softag 3 antibody, nanobody, streptactin, anti-T7-tag antibody, FlAsH biarsenical compounds, ReAsH biarsenical compounds, anti-Ty1 tag antibody, anti-V5 tag antibody, anti-VSV tag antibody, anti-Xpress tag antibody, pilin-C protein, SpyCatcher protein, SnoopCatcher protein, SnoopTagJr protein, SdyCatcher protein, glutathione, GFP-antibody, haloalkane substrate, benzylguanine derivatives, benzylcytosine derivatives, HUH specific DNA sequence, amylose agarose, Nus-tag antibody, anti-thioredoxin-tag antibody, protein-A sepharose, lactose, agarose, and sepharose, optionally the secondarytarget binding moiety is selected from avidin and streptavidin.

79. The method of any one of claims 52 to 78, wherein the reporter enzyme is selected from a phosphatase, optionally alkaline phosphatase, lyase, hydrolase, synthase, synthetase, oxidoreductase, dehydrogenase, oxidase, transferase, isomerase, ligase, protease, such as trypsin, proteinase, peroxidase, glucose oxidase, myeloperoxidase, oxidase, monooxygenase, cytochrome, decarboxylase, lipase, caspase, amylase, peptidase, transaminase, kinase activity, DNA or RNA polymerase, optionally TAQ, restriction enzyme, klenow fragment, and DNA ligase.

80. The method of claim 79, wherein the reporter enzyme is selected from alkaline phosphatase, horseradish peroxidase, trypsin, cytochrome C monooxygenase, and myeloperoxidase.

81. The method of any one of claims 52 to 80, wherein the one or more ionizable products are readily ionizable under ESI-MS/MS or MALDI-TOF and generates a product ion characterized by a high signal to noise ratio, and the substrate is optionally selected from:
a. a phosphorylated nucleoside, optionally AMP or CMP, or nucleotide, optionally ATP or CTP, phosphorylated alkaloid, phosphorylated amino acid, phosphorylated amino acid polymer, and phosphorylated metabolite when the enzyme is alkaline phosphatase (AP);
b. a compound selected from phenols, amines, optionally phenolic amines, aromatic compounds, olefin halogenations, luminol, pyrogallol, 2,2′-azino-bis(3-ethylbenzothiazoline-6-sulphonic acid (ABTS), and Amplex® Red when the reporter enzyme is horseradish peroxidase (HRP); or from
c. opiates, detergents, dye precursor, alcohols, and matrix.

82. The method of claim 79, wherein the reporter enzyme substrate is selected from pyridoxamine-5-phosphate (PA5P), p-nitrophenyl phosphate (PNPP), Amplex® Red (AR), naphthol ASMX phosphate, luminol, Lumigen® TMA3, Lumigen® TMA6, sphingosine, 4MUP, L-(+)-2-amino-6-phosphonohexanoic acid, 5-bromo-4-chloro-3-indolyl phosphate (BCIP), BluePhos®, phenylbenzene ω phosphono-α-amino acid, O-phospho-DL-threonine, adenosine monophosphate (AMP), AR (3-amino-9-ethylcarbazole), 4-CN (4-chloro-1-naphtol), DAB (3,3′-DiAminoBenzimidine), OPD (o-phenylene diamine), TMB (3,3″,5,5″-tetramethylbenzidine), pNPP (p-nitrophenyl phosphate), NBT (nitroblue tetrazolium), INT (p-iodonitrotetrazolium), MUP (4-methylumbelliferyl phosphate), and FDP fluorescein diphosphate), pyrogallol.

83. The method of claim 82, wherein the reporter enzyme substrate is selected from:
a. AR, luminol, Lumigen® TMA3, and Lumigen® TMA6, when the reporter enzyme detection probe comprises HRP; or from
b. naphthol ASMX phosphate, and PNPP, when the reporter enzyme detection probe comprises AP.

84. The method of any one of claims 52 to 83, wherein the substrate reaction solution comprises a non-ionic non polymeric detergent, optionally selected from N-octylglucoside, deoxycholate, rapigest, octyl-beta-glucopyranoside, octylglucopyranoside, chaps, big chap, non-ionic acid labile surfactants, glucosides, n-Octyl-β-D-glucopyranoside, n-Nonyl-β-D-glucopyranoside thioglucosides, n-Octyl-β-D-thioglucopyranoside maltosides, n-Decyl-β-D-maltopyranoside, n-Dodecyl-β-D-maltopyranoside, n-Undecyl-β-D-maltopyranoside, n-Tridecyl-β-D-maltopyranoside, cymal-5, cymal-6, thiomaltosides, n-Dodecyl-β-D-thiomaltopyranoside, alkyl glycosides, octyl glucose neopentyl glycol, polyoxyethylene glycols, triton, NP40, Tween™, Tween™ 20, Triton X-100, triton x-45, C₈E4, C8E5, C10E5, C12E8, C12E9, Brij, Anapoe-58, Brij-58, and combinations thereof.

85. The method of any one of claims 52 to 84, wherein the substrate reaction solution further comprises 4-iodophenylboronic acid when the substrate comprises luminol.

86. The method of any one of claims 52 to 85, wherein the solid phase is a reaction vessel optionally a bead, a plate, a capillary, a filter, or a nano/micro/milli well reaction vessel, and wherein the surface is selected from paper, nitrocellulose, acrylate, plastic, polystyrene, polyvinylene fluoride (PVDF), melamine, silica, polylysine coated glass, 3-aminopropyl-triethoxysilane (APTES) treated glass, and 3-aminopropyl-trimethoxysilane (APTMS) treated glass.

87. The method of any one of claims 56 to 86, wherein the attaching of the capture oligonucleotide probe to the solid phase is through H-hydroxysuccinimide (NHS), N-oxysuccinimide (NOS), maleimide, hydrazide, orglutaraldehyde coupling.

88. The method of any one of claims 52 to 87, wherein the amplified nucleic acid product or the amplified nucleic acid product:reporter enzyme complex is incubated with the reporter enzyme substrate in the substrate reaction solution to generate the one or more ionizable products for a period of time less than 72 hours, less than 24 hours, less than 12 hours, less than 60 minutes, less than 50 minutes, less than 40 minutes, less than 30 minutes, less than 20 minutes, less than 15 min, less than 10 min, less than 5 min, less than 2 min, or less than 1 min.

89. The method of any one of claims 76 to 88, wherein the product ion is assayed by SIM and/or SRM using an optimized fragmentation energy and m/z range.

90. The method of claim 81, wherein the substrate is AMP, ADP or ATP and one or the ionizable products generated comprises adenosine, the product ion of which is assayed by SIM at 268 m/z; or the substrate is CMP, CDP or CTP and one or the ionizable products generated comprises cytosine, the product ion of which is assayed by SIM at 283 m/z; or the substrate is AR and one of the one or more ionizable products generated comprises resorufin, the product ion of which is assayed by SIM at 214 m/z and SRM using the major intense fragment at 214-186 m/z.

91. The method of claim 81, wherein the substrate is naphthol ASMX phosphate and one of the one or more ionizable products generated comprises dephosphorylated naphthol ASMX, the product ion of which is assayed by SIM at 292 m/z and SRM using the major intense fragment at 292-171 m/z or the substrate is PA5P and one or the ionizable products generated comprises PA, the product ion of which is assayed by SIM at 169 m/z.

92. The method of any one of claims 52 to 91, wherein the ionizable products are ionized to product ions in ionization solution.

93. The method of any one of claims 52 to 91, wherein the test sample is a biological sample, industrial product, or environmental sample.

94. The method of claim 93, wherein the biological sample is a blood sample, urine sample, fecal sample, effusate, tissue sample or sputum sample.

95. The method of any one of claims 52 to 93, wherein the PCR is selected from real time PCR (rtPCR), quantitative PCR (qPCR), reverse transcription PCR, nested PCR, hybridization chain reaction, rolling circle PCR, and substrate recycling reaction.

96. A method of quantifying the amount of a target nucleic acid molecule in a test sample comprising the steps:
a. detecting the target nucleic acid molecule according to the method of anyone of claims 1-51; and
b. quantifying the amount of target nucleic acid molecule in the test sample based on the intensity of the signal for one or more of the ionizable products detected by mass spectrometry.

97. The method of claim 96 wherein the quantification comprises comparing the intensity of the signal for one or more products against signal intensities generated using known quantities of the target nucleic acid molecule, under similar conditions.

98. The method of claim 96 or 97, wherein the target nucleic acid molecule is present or suspected to be present in the sample in or up to a pico mol, femto mol, or atto mol range.

99. The method of any one of claims 1 to 98, wherein one or more target oligonucleotide templates are detected.

100. The method of any one of claims 1 to 99, wherein the target nucleic acid molecule is a plasmid DNA or a sequence comprised in a bacterial, viral, fungal, mammalian or plant genome.

101. The method of claim 100, wherein the bacterial genome is selected from E. coli, Staphylococcus aureus, Chlamydia, Vibrio cholera, Clostridium, Enterococci, Fusobacterium, anaerobic bacilli, Gram negative cocci, Gram positive bacilli, Haemophilus, Haemophilus influenza, Klebsiella, Lactobacillus, Listeria, Borrelia, Mycobacterium, Mycoplasma, Neisseria, Prevotella, Pseudomonas, Salmonella, Shigella, Spirochaetes, Staphylococcus, Streptococcus, and Yersinia genome, optionally the bacterial genome is selected from E. coli, and Staphylococcus aureus, and/or wherein the fungal genome is selected from Candida genome.

102. The method of claim 100, wherein the viral genome is selected from HIV, SARS-CoV, MERS, SARS-CoV-2, Ebola virus, influenza virus, coronavirus genome, Enteroviruses, Hepatitis virus, Herpes virus, HPV, Noroviruses, Parainfluenza, Rhinoviruses, and Varicella Virus genome, optionally the viral genome is selected from HIV, SARS-CoV, MERS, SARS-CoV-2, Ebola virus, influenza virus, and coronavirus genome.

103. The method of claim 100, wherein the mammalian genome is a human genome.

104. The method of claim 100, wherein the target nucleic acid molecule has a sequence comprised in the HIV genome.

105. The method of claim 100, wherein the target nucleic acid molecule has a sequenced comprised in the SARS-CoV-2 genome.

106. A method of detecting HIV comprising a method as defined in any one of claims 1 to 51, wherein the target nucleic acid molecule is a HIV nucleic acid molecule.

107. The method of claim 106, wherein the capture oligonucleotide probe has a sequence selected from SEQ ID No. 14, SEQ ID No 17, SEQ ID No 20, and SEQ ID No 23.

108. The method of claim 106 or 107, wherein the detection oligonucleotide probe oligonucleotide has a sequence selected from SEQ ID No. 16, SEQ ID No 19, SEQ ID No 22, and SEQ ID No. 25.

109. A method of detecting SARS-CoV-2 comprising a method as defined in any one of claims 1 to 51, wherein the target nucleic acid molecule is a SARS-CoV-2 nucleic acid molecule.

110. The method of claim 109, wherein the capture oligonucleotide probe has a sequence selected from SEQ ID No. 6, and SEQ ID No. 13.

111. The method of claim 109 or 110, wherein the detection oligonucleotide probe oligonucleotide has a sequence selected from SEQ ID No. 5, and SEQ ID No. 12.

112. A method of detecting HIV comprising a method as defined in any one of claims 52 to 98, wherein the target nucleic acid molecule is a HIV nucleic acid molecule.

113. The method of claim 112 comprising a method as defined in any one of claims 56 to 98 when claims 57 to 98 depend on claim 56, wherein the capture oligonucleotide probe has a sequence selected from SEQ ID No. 14, SEQ ID No 17, SEQ ID No 20, and SEQ ID No 23.

114. A method of detecting SARS-CoV-2 comprising a method as defined in any one of claims 52 to 98, wherein the target nucleic acid molecule is a SARS-CoV-2 nucleic acid molecule.

115. The method of claim 114, wherein the modified primer has a sequence selected from SEQ ID No. 2, SEQ ID No. 3, SEQ ID No. 7, SEQ ID No. 8, SEQ ID No. 9, and SEQ ID No. 10.

116. The method of claim 114, wherein the second primer has a sequence selected from SEQ ID No. 2, SEQ ID No. 3, SEQ ID No. 7, SEQ ID No. 8, SEQ ID No. 9, and SEQ ID No. 10.

117. The method of claim 114, wherein the modified primer has sequence of SEQ ID No. 2, and the second primer has sequence of SEQ ID No. 3, or SEQ ID No 8.

118. The method of claim 114, wherein the modified primer has sequence of SEQ ID No. 3, and the second primer has sequence of SEQ ID No. 2, or SEQ ID No. 7.

119. The method of claim 114, wherein the modified primer has sequence of SEQ ID No.7, and the second primer has sequence of SEQ ID No 3, or SEQ ID No. 8.

120. The method of claim 114, wherein the modified primer has sequence of SEQ ID No. 8, and the second primer has sequence of SEQ ID No 2, SEQ ID No. 7.

121. The method of claim 114, wherein the modified primer has sequence of SEQ ID No. 9, and the second primer has sequence of SEQ ID No.10.

122. The method of claim 114, wherein the modified primer has sequence of SEQ ID No. 10, and the second primer has sequence of SEQ ID No.9.

123. The method of claim 111141 comprising a method as defined in any one of claims 56 to 98 when claims 57 to 98 depend on claim 56, wherein the capture oligonucleotide probe has a sequence selected from SEQ ID No. 6, and SEQ ID No. 13.

124. A kit comprising:
i. a capture oligonucleotide probe, the capture oligonucleotide probe optionally bound of a solid phase, optionally through a linker;
ii. a volatile binding solution comprising a volatile buffer and being substantially free of NaCl, or a cross-linking agent;
iii. a detection oligonucleotide probe, the detection oligonucleotide probe comprising an oligonucleotide and a secondary target moiety;
iv. a reporter enzyme detection probe, the reporter enzyme detection probe comprising a reporter enzyme and a secondary target binding moiety capable of binding the secondary target moiety; and/or
v. one or more of: a substrate, a solid phase, a standard, optionally a product ion standard, optionally for preparing a standard curve or tuning calibrant, a second binding solution, a third binding solution, a substrate reaction solution, ionization solution, quenching solution, optionally a second binding solution, detection probe solution, substrate reaction solution, quenching solution, ionization solution as defined in any one of claims 1 to 51.

125. The kit of claim 124, wherein the second binding buffer and the substrate reaction buffer each are volatile and each independently comprise a volatile buffer.

126. The kit of claim 125, wherein the volatile buffer is ethanolamine, ammonium bicarbonate, ammonium formate, pyridinium formate, trialkylammonium/formic acid, ammonium acetate, trialkylammonium bicarbonate, N-ethylmorpholine/acetate, trialkylammonium acetate, and combinations thereof.

127. The kit of claim 125 or 126, wherein the volatile buffer is selected from ethanolamine, ammonium acetate, trialkylammonium bicarbonate, and combinations thereof.

128. The kit of claim 126 or 127, wherein the trialkylammonium is selected from trimethylammonium, triethylammonium, and combinations thereof.

129. The kit of any one of claims 125 to 127, wherein the volatile buffer is ethanolamine.

130. The kit of any one of claims 124 to 129, wherein the capture oligonucleotide probe and the detection oligonucleotide probe both bind a target nucleic acid molecule.

131. The kit of claim 130, wherein the target nucleic acid molecule has a sequence comprised in a bacterial, viral, fungal, mammalian or plant genome.

132. The kit of any one of claims 124 to 131, wherein the enzyme of the reporter enzyme detection probe is selected from alkaline phosphatase, horseradish peroxidase, trypsin, cytochrome C monooxygenase, and myeloperoxidase.

133. The kit of any one of claims 124 to 132, wherein the substrate is selected from adenosine monophosphate (AMP), CMP, ATP, CMP, PSAP, p-nitrophenyl phosphate (PNPP), Amplex® Red (AR), naphthol ASMX phosphate, luminol, Lumigen® TMA3, Lumigen® TMA6, sphingosine, 4MUP, L-(+)-2-amino-6-phosphonohexanoic acid, 5-Bromo-4-chloro-3-indolyl phosphate (BCIP), BluePhos®, phenylbenzene ω phosphono-α-amino acid, O-phospho-DL-threonine, AR (3-amino-9-ethylcarbazole), 4-CN (4-Chloro-1-Naphtol), DAB (3,3′-DiAminoBenzimidine), OPD (o-Phenylene Diamine), TMB (3,3″,5,5″-tetramethylbenzidine), pNPP (p-Nitrophenyl Phosphate), NBT (nitroblue tetrazolium), INT (p-iodonitrotetrazolium), MUP (4-Methylumbelliferyl Phosphate), FDP (Fluorescein DiPhosphate), and pyrogallol.

134. The kit of any one of claims 124 to 133, wherein the ionization solution comprises an acid or a base, optionally selected from formic acid, acetic acid, trifluoroacetic acid. ammonium hydroxide, methylamine, ethylamine, orpropylamine.

135. The kit of any one of claims 124 to 134, wherein the quenching solution comprises optionally 50% Acetonitrile, 0.1% Acetic acid or 0.1% formic acid or 0.1% trifluoroacetic acid for positive ionization or 0.1% ammonium hydroxide for negative ionization.

136. The kit of any one of claims 124 to 135, wherein the secondary target moiety is selected from biotin, ALFA-tag, AviTag, C-tag, Calmoudulin-Tag, Polyglutamate Tag, E-Tag, Flag-tag, HA-tag, His-Tag, myc-Tag, NE-tag, Rho1D4-Tag, S-Tag, SBP-Tag, Softag 1, Softag 3, Spot-tag, Strept-tag, T7-tag, TC-tag, Ty1 tag, V5 tag, VSV-tag, Xpress tag, Isopeptag, SpyTag, SnoopTag, DogTag, Sdy Tag, Biotin carboxyl carrier protein, glutathione-S-transferase tas, GFP tag, HaloTag, SNAP-tag, CLIP-tag, HUH-Tag, Maltose-binding protein tag, Nus-tag, thioredoxin-tag, Fc-tag, and CRDSAT-tag, optionally the second target moiety is biotin.

137. The kit of any one of claims 124 to 136, wherein the secondary target binding moiety is selected from avidin, streptavidin, calmodulin, anion-exchange resin, Mono-Q, cation-exchange resin, anti-E-tag antibody, anti-FLAG-tag antibody, anti-HA-tag antibody, nickel or cobalt chelate, anti-Myc-tag antibody, anti-NE-tag antibody, anti-Rho1D4-tag antibody, anti-S-tag antibody, anti-Softag 1 antibody, anti-Softag 3 antibody, nanobody, streptactin, anti-T7-tag antibody, FlAsH biarsenical compounds, ReAsH biarsenical compounds, anti-Ty1 tag antibody, anti-V5 tag antibody, anti-VSV tag antibody, anti-Xpress tag antibody, pilin-C protein, SpyCatcher protein, SnoopCatcher protein, SnoopTagJr protein, SdyCatcher protein, glutathione, GFP-antibody, haloalkane substrate, benzylguanine derivatives, benzylcytosine derivatives, HUH specific DNA sequence, amylose agarose, Nus-tag antibody, anti-thioredoxin-tag antibody, protein-A sepharose, lactose, agarose, and sepharose, optionally the secondary target binding moiety is selected from avidin and streptavidin.

138. The kit of claim 136 or 137 wherein the detection oligonucleotide probe is a biotinylated oligonucleotide probe comprising a sequence complimentary to a portion of the target nucleic acid molecule.

139. The kit of claim 137 or 138, wherein the reporter enzyme detection probe is alkaline phosphatase streptavidin (APSA) enzyme.

140. The kit of any one of claims 134 to 139, wherein the capture oligonucleotide probe comprises a sequence selected from SEQ ID No. 6, SEQ ID No. 13, SEQ ID No. 14, SEQ ID No 17, SEQ ID No 20, SEQ ID No 23, SEQ ID No 26, SEQ ID No 29, SEQ ID No 32, and SEQ ID No 35.

141. The kit of any one of claims 124 to 139, wherein the oligonucleotide of the detection oligonucleotide probe comprises a sequence selected from SEQ ID No. 5, SEQ ID No. 12, SEQ ID No. 16, SEQ ID No 19, SEQ ID No 22, SEQ ID No 25, SEQ ID No 28, SEQ ID No 31, SEQ ID No 34, and SEQ ID No 37.

142. The kit of any one of claims 124 to 139, wherein the capture oligonucleotide probe comprises a sequence of SEQ ID No 14, and the oligonucleotide of the detection oligonucleotide probe has a sequence of SEQ ID No. 16.

143. The kit of any one of claims 124 to 139, wherein the capture oligonucleotide probe comprises a sequence of SEQ ID No. 6, and the oligonucleotide of the detection oligonucleotide probe has a sequence of SEQ ID No. 5.

144. The kit of any one of claims 124 to 139, wherein the capture oligonucleotide probe comprises a sequence of SEQ ID No. 13, and the oligonucleotide of the detection oligonucleotide probe has a sequence of SEQ ID No.12.

145. The kit of any one of claims 124 to 139, wherein the capture oligonucleotide probe comprises a sequence of SEQ ID No 17, and the oligonucleotide of the detection oligonucleotide probe has a sequence of SEQ ID No. 19.

146. The kit of any one of claims 124 to 139, wherein the capture oligonucleotide probe comprises a sequence of SEQ ID No 20, and the oligonucleotide of the detection oligonucleotide probe has a sequence of SEQ ID No. 22.

147. The kit of any one of claims 124 to 139, wherein the capture oligonucleotide probe comprises a sequence of SEQ ID No 23, and the oligonucleotide of the detection oligonucleotide probe has a sequence of SEQ ID No. 25.

148. The kit of any one of claims 124 to 139, wherein the capture oligonucleotide probe comprises a sequence of SEQ ID No 26, and the oligonucleotide of the detection oligonucleotide probe has a sequence of SEQ ID No. 28.

149. The kit of any one of claims 124 to 139, wherein the capture oligonucleotide probe comprises a sequence of SEQ ID No 29, and the oligonucleotide of the detection oligonucleotide probe has a sequence of SEQ ID No. 31.

150. The kit of any one of claims 124 to 139, wherein the capture oligonucleotide probe comprises a sequence of SEQ ID No 32, and the oligonucleotide of the detection oligonucleotide probe has a sequence of SEQ ID No. 34.

151. The kit of any one of claims 124 to 139, wherein the capture oligonucleotide probe comprises a sequence of SEQ ID No 35, and the oligonucleotide of the detection oligonucleotide probe has a sequence of SEQ ID No. 37.

152. A kit comprising:
i. a modified primer, the modified primer being functionalized with a secondary target moiety or a reporter enzyme;
ii. a second primer;
iii. when the modified primer is functionalized with the secondary target moiety, a reporter enzyme detection probe, the reporter enzyme detection probe comprising a reporter enzyme and a secondary target binding moiety capable of binding the secondary target moiety; and
iv. one or more of: a substrate, a solid phase, a standard, optionally a product ion standard, optionally for preparing a standard curve or tuning calibrant, a binding solution, a second binding solution, a substrate reaction solution, ionization solution, quenching solution, optionally a binding solution, second binding solution, detection probe solution, washing solution, substrate reaction solution, quenching solution, ionization solution as defined in any one of claims 1 to 95,
wherein when the modified primer is a forward primer, the second primer is a reverse primer, and when the modified primer is a reverse primer, the second primer is a forward primer.

153. The kit of claim 152, wherein the second primer is attached to the solid phase.

154. The kit of claim 152, wherein the solid phase is attached to a capture oligonucleotide probe, optionally through a linker.

155. The kit of any one of claims 152 to 154, wherein the binding solution and the second binding solution are each independently volatile and substantially free of NaCl.

156. The kit of claim 155, wherein the binding solution, the second binding solution and the washing solution each independently comprises a volatile buffer.

157. The kit of claim 156, wherein the volatile buffer is ethanolamine, ammonium bicarbonate, ammonium formate, pyridinium formate, trialkylammonium/formic acid, ammonium acetate, trialkylammonium bicarbonate, N-ethylmorpholine/acetate, trialkylammonium acetate, and combinations thereof.

158. The kit of claim 156 or 157, wherein the volatile buffer is selected from ethanolamine, ammonium acetate, trialkylammonium bicarbonate, and combinations thereof.

159. The kit of claim 157 or 158, wherein the trialkylammonium is selected from trimethylammonium, triethylammonium, and combinations thereof.

160. The kit of any one of claims 155 to 159, wherein the volatile buffer is ethanolamine.

161. The kit of any one of claims 152 to 160, wherein the enzyme of the reporter enzyme detection probe is selected from alkaline phosphatase, horseradish peroxidase, trypsin, cytochrome C monooxygenase, and myeloperoxidase.

162. The kit of any one of claims 152 to 161, wherein the substrate is selected from adenosine monophosphate (AMP), CMP, ATP, CMP, PSAP, p-nitrophenyl phosphate (PNPP), Amplex® Red (AR), naphthol ASMX phosphate, luminol, Lumigen® TMA3, Lumigen® TMA6, sphingosine, 4MUP, L-(+)-2-amino-6-phosphonohexanoic acid, 5-Bromo-4-chloro-3-indolyl phosphate (BCIP), BluePhos®, phenylbenzene ω phosphono-α-amino acid, O-phospho-DL-threonine, AR (3-amino-9-ethylcarbazole), 4-CN (4-Chloro-1-Naphtol), DAB (3,3′-DiAminoBenzimidine), OPD (o-Phenylene Diamine), TMB (3,3″,5,5″-tetramethylbenzidine), pNPP (p-Nitrophenyl Phosphate), NBT (nitroblue tetrazolium), INT (p-iodonitrotetrazolium), MUP (4-Methylumbelliferyl Phosphate), FDP (Fluorescein DiPhosphate), and pyrogallol.

163. The kit of any one of claims 152 to 162, wherein the ionization solution comprises an acid or a base, optionally selected from formic acid, acetic acid, trifluoroacetic acid. ammonium hydroxide, methylamine, ethylamine, or propylamine.

164. The kit of any one of claims 152 to 163 wherein the quenching solution comprises optionally 50% Acetonitrile, 0.1% Acetic acid or 0.1% formic acid or 0.1% trifluoroacetic acid for positive ionization or 0.1% ammonium hydroxide for negative ionization.

165. The kit of any one of claims 152 to 164, wherein the secondary target moiety is selected from biotin, ALFA-tag, AviTag, C-tag, Calmoudulin-Tag, Polyglutamate Tag, E-Tag, Flag-tag, HA-tag, His-Tag, myc-Tag, NE-tag, Rho1D4-Tag, S-Tag, SBP-Tag, Softag 1, Softag 3, Spot-tag, Strept-tag, T7-tag, TC-tag, Ty1 tag, V5 tag, VSV-tag, Xpress tag, Isopeptag, SpyTag, SnoopTag, DogTag, Sdy Tag, Biotin carboxyl carrier protein, glutathione-S-transferase tas, GFP tag, HaloTag, SNAP-tag, CLIP-tag, HUH-Tag, Maltose-binding protein tag, Nus-tag, thioredoxin-tag, Fc-tag, and CRDSAT-tag, optionally the second target moiety is biotin.

166. The kit of any one of claims 152 to 165, wherein the secondary target binding moiety is selected from avidin, streptavidin, calmodulin, anion-exchange resin, Mono-Q, cation-exchange resin, anti-E-tag antibody, anti-FLAG-tag antibody, anti-HA-tag antibody, nickel or cobalt chelate, anti-Myc-tag antibody, anti-NE-tag antibody, anti-Rho1D4-tag antibody, anti-S-tag antibody, anti-Softag 1 antibody, anti-Softag 3 antibody, nanobody, streptactin, anti-T7-tag antibody, FlAsH biarsenical compounds, ReAsH biarsenical compounds, anti-Ty1 tag antibody, anti-V5 tag antibody, anti-VSV tag antibody, anti-Xpress tag antibody, pilin-C protein, SpyCatcher protein, SnoopCatcher protein, SnoopTagJr protein, SdyCatcher protein, glutathione, GFP-antibody, haloalkane substrate, benzylguanine derivatives, benzylcytosine derivatives, HUH specific DNA sequence, amylose agarose, Nus-tag antibody, anti-thioredoxin-tag antibody, protein-A sepharose, lactose, agarose, and sepharose, optionally the secondary target binding moiety is selected from avidin and streptavidin.

167. The kit of claim 165 or 166, wherein the reporter enzyme detection probe is alkaline phosphatase streptavidin (APSA) enzyme.

168. The kit of any one of claims 152 to 167, wherein the modified primer and the second primer are primers for a target nucleic acid molecule that has a sequence comprised in a bacterial, viral, fungal, mammalian or plant genome.

169. The kit of any one of claims 152 to 168, wherein the modified primer has a sequence selected from SEQ ID No. 2, SEQ ID No. 3, SEQ ID No. 7, SEQ ID No. 8, SEQ ID No. 9, and SEQ ID No. 10.

170. The kit of any one of claims 152 to 168, wherein the second primer has a sequence selected from SEQ ID No. 2, SEQ ID No. 3, SEQ ID No. 7, SEQ ID No. 8, SEQ ID No. 9, and SEQ ID No. 10.

171. The kit of any one of claims 152 to 168, wherein the modified primer has sequence of SEQ ID No. 2, and the second primer has sequence of SEQ ID No. 3, or SEQ ID No 8.

172. The kit of any one of claims 152 to 168, wherein the modified primer has sequence of SEQ ID No. 3, and the second primer has sequence of SEQ ID No. 2, or SEQ ID 7.

173. The kit of claim 171 or 172, wherein the capture oligonucleotide has sequence of SEQ ID No. 6.

174. The kit of any one of claims 152 to 168, wherein the modified primer has sequence of SEQ ID No.7, and the second primer has sequence of SEQ ID No. 8.

175. The kit of any one of claims 152 to 168, wherein the modified primer has sequence of SEQ ID No. 8, and the second primer has sequence of SEQ ID No.7.

176. The kit of any one of claims 152 to 168, wherein the modified primer has sequence of SEQ ID No. 9, and the second primer has sequence of SEQ ID No.10.

177. The kit of any one of claims 152 to 168, wherein the modified primer has sequence of SEQ ID No. 10, and the second primer has sequence of SEQ ID No.9.

178. The kit of claim 176 or 177, wherein the capture oligonucleotide has sequence of SEQ ID No. 13.

179. A nucleic acid of sequence selected from SEQ ID No. 2 to 46.

180. The nucleic acid of claim 179, wherein the nucleic acid is attached to a solid support, optionally a solid support as defined in claim 86 or 87.

181. The nucleic acid of claim 179, wherein the nucleic acid is attached to a second target moiety as defined in claim 77.


SEQUENCE LISTING (75 sequences)

SEQ ID NO: 1 - DNA, 1260 bp, Artificial Sequence
  SARS COV-2 NUCLEOCAPSID
atgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaa

SEQ ID NO: 2 - DNA, 22 bp, Artificial Sequence
  SARS COV-2 PRIMER
caaaacaacgtcggccccaagg

SEQ ID NO: 3 - DNA, 24 bp, Artificial Sequence
  SARS COV-2 PRIMER
ggtcatctggactgctattggtgt

SEQ ID NO: 4 - DNA, 138 bp, Artificial Sequence
  SARS COV-2 TARGET SEQUENCE
caaaacaacgtcggccccaaggtttacccaataatactgcgtcttggttcaccgctctcactcaacatggcaaggaagaccttaaattccctcgaggacaaggcgttccaattaacaccaatagcagtccagatgacc

SEQ ID NO: 5 - DNA, 50 bp, Artificial Sequence
  SARS COV-2 DETECTION PROBE
gaaccaagacgcagtattattgggtaaaccttggggccgacgttgttttg

SEQ ID NO: 6 - DNA, 50 bp, Artificial Sequence; identical to SEQ ID NO: 5
  CAPTURE OLIGONUCLEOTIDE PROBE SEQUENCE

SEQ ID NO: 7 - DNA, 20 bp, Artificial Sequence
  SARS CoV2 Set 3 PCR Primers
tggaccccaaaatcagcgaa

SEQ ID NO: 8 - DNA, 20 bp, Artificial Sequence
  SARS CoV2 Set 3 PCR Primers
tgccgtctttgttagcacca

SEQ ID NO: 9 - DNA, 20 bp, Artificial Sequence
  SARS CoV2 Set 3 PCR Primers
cgaggacaaggcgttccaat

SEQ ID NO: 10 - DNA, 20 bp, Artificial Sequence
  SARS CoV2 Set 3 PCR Primers
tgttagcaggattgcgggtg

SEQ ID NO: 11 - DNA, 224 bp, Artificial Sequence
  Target nucleic acid molecule for SARS-CoV-2
gcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcac

SEQ ID NO: 12 - DNA, 50 bp, Artificial Sequence
  SARS COV-2 Detection probe oligonucleotide
gcatcatatgggttgcaactgagggagccttgaatacaccaaaagatcac

SEQ ID NO: 13 - DNA, 50 bp, Artificial Sequence
  Capture oligonucleotide probe sequence
gcgttccaattaacaccaatagcagtccagatgaccaaattggctactac

SEQ ID NO: 14 - DNA, 47 bp, Artificial Sequence
  HIV Capture oligonucleotide probe
ctttccgctggggactttccagggaggcgtggcctgggcgggactgc

SEQ ID NO: 15 - DNA, 93 bp, Artificial Sequence
  HIV Target nucleic acid molecule
cagtcccgcccaggccacgcctccctggaaagtccccagcggaaagtcccttgtagcaagctcgatgtcagcagttcttgaagtactccggat

SEQ ID NO: 16 - DNA, 45 bp, Artificial Sequence
  HIV Detection oligonucleotide probe
gatccggagtacttcaagaactgctgacatcgagcttgctacaag

SEQ ID NO: 17 - DNA, 50 bp, Artificial Sequence
  HIV Capture oligonucleotide probe
aagttcttctgatcctgtctgaagggatggttgtaaatgccctattattc

SEQ ID NO: 18 - DNA, 126 bp, Artificial Sequence
  HIV Target nucleic acid molecule
gaataatagggcatttacaaccatcccttcagacaggatcagaagaacttaaatcattatataatttagtagcagtcctttattgttattgtgtgcatcaaaggatagaggtaaaagacaccaatg

SEQ ID NO: 19 - DNA, 50 bp, Artificial Sequence
  Detection oligonucleotide probe
cattggtgtcttttacctctatcctttgatgcacacaataacaataaagg

SEQ ID NO: 20 - DNA, 50 bp, Artificial Sequence
  HIV Capture oligonucleotide probe
tggggtggccccttctgataatgctgtaaacatgggtattacttctgggc

SEQ ID NO: 21 - DNA, 142 bp, Artificial Sequence
  HIV Target nucleic acid molecule
gcccagaagtaatacccatgtttacagcattatcagaaggggccaccccacaagatttaaacaccatgttaaacacagtggggggacatcaagcagccatgcaaatgttaaaagagaccatcaatgaggaagctgcagaatg

SEQ ID NO: 22 - DNA, 50 bp, Artificial Sequence
  HIV Detection oligonucleotide probe
cattctgcagcttcctcattgatggtctcttttaacatttgcatggctgc

SEQ ID NO: 23 - DNA, 50 bp, Artificial Sequence
  HIV Capture oligonucleotide probe
aatcccaggattatccatcttttatagatttctcctactgggataggtgg

SEQ ID NO: 24 - DNA, 133 bp, Artificial Sequence
  HIV Target nucleic acid molecule
ccacctatcccagtaggagaaatctataaaagatggataatcctgggattaaataaaatagtaagaatgtatagccctaccagcattctggacataagacaaggaccaaaagaaccctttagagactatgtag

SEQ ID NO: 25 - DNA, 50 bp, Artificial Sequence
  HIV Detection oligonucleotide probe
ctacatagtctctaaagggttcttttggtccttgtcttatgtccagaatg

SEQ ID NO: 26 - DNA, 50 bp, Artificial Sequence
  SHIGA TOXIN E COLI (STEC) Capture oligonucleot ide probe
tatatgttcaagaggggtcgatatctctgtccgtatactatttaacgaag

SEQ ID NO: 27 - DNA, 138 bp, Artificial Sequence
  STEC Target nucleic acid molecule
cttcgttaaatagtatacggacagagatatcgacccctcttgaacatatatctcaggggaccacatcggtgtctgttattaaccacaccccaccgggcagttattttgctgtggatatacgagggcttgatgtctatc

SEQ ID NO: 28 - DNA, 50 bp, Artificial Sequence
  STEC Detection oligonucleotide probe
gatagacatcaagccctcgtatatccacagcaaaataactgcccggtggg

SEQ ID NO: 29 - DNA, 50 bp, Artificial Sequence
  STEC Capture oligonucleotide probe
attcagtataacggccacagtccccagtatcgctgatatattattaaagg

SEQ ID NO: 30 - DNA, 121 bp, Artificial Sequence
  STEC Target nucleic acid molecule
cctttaataatatatcagcgatactggggactgtggccgttatactgaattgccatcatcagggggcgcgttctgttcgcgccgtgaatgaagagagtcaaccagaatgtcagataactgg

SEQ ID NO: 31 - DNA, 50 bp, Artificial Sequence
  STEC Detection oligonucleotide probe
ccagttatctgacattctggttgactctcttcattcacggcgcgaacaga

SEQ ID NO: 32 - DNA, 50 bp, Artificial Sequence
  STEC Capture oligonucleotide probe
cagcgactggtccagtattctttcccgtcaaccttcactgtaaatgtgtc

SEQ ID NO: 33 - DNA, 130 bp, Artificial Sequence
  STEC Target nucleic acid molecule
gacacatttacagtgaaggttgacgggaaagaatactggaccagtcgctggaatctgcaaccgttactgcaaagtgctcagttgacaggaatgactgtcacaatcaaatccagtacctgtgaatcaggct

SEQ ID NO: 34 - DNA, 50 bp, Artificial Sequence
  STEC Detection oligonucleotide probe
agcctgattcacaggtactggatttgattgtgacagtcattcctgtcaac

SEQ ID NO: 35 - DNA, 50 bp, Artificial Sequence
  S AUREUS Capture oligonucleotide probe
catgaaaagttgattgccatataccgggttccaagaatctctatcatatg

SEQ ID NO: 36 - DNA, 144 bp, Artificial Sequence
  S AUREAS Target nucleic acid molecule
catatgatagagattcttggaacccggtatatggcaatcaacttttcatgaaaactagaaatggttctatgaaagcagcagagaacttccttgatcctaacaaagcaagttctctattatcttcagggttttcaccagacttcg

SEQ ID NO: 37 - DNA, 50 bp, Artificial Sequence
  S AUREUS Detection oligonucleotide probe
cgaagtctggtgaaaaccctgaagataatagagaacttgctttgttagga

SEQ ID NO: 38 - DNA, 20 bp, Artificial Sequence
  HIV forward primer
ccaggccagatgagagaacc

SEQ ID NO: 39 - DNA, 20 bp, Artificial Sequence
  HIV reverse primer
tgaagcttgctcggctctta

SEQ ID NO: 40 - DNA, 258 bp, Artificial Sequence
  HIV target nucleic acid molecule
ccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaagaaccctttagagactatgtagaccggttctataaaactctaagagccgagcaagcttca

SEQ ID NO: 41 - DNA, 36 bp, Artificial Sequence
  HIV FORWARD PRIMER
ccacctatcccagtaggagaaatctataaaagatgg

SEQ ID NO: 42 - DNA, 36 bp, Artificial Sequence
  HIV REVERSE PRIMER
ctacatagtctctaaagggttcttttggtccttgtc

SEQ ID NO: 43 - DNA, 133 bp, Artificial Sequence
  HIV  target nucleic acid molecule
ccacctatcccagtaggagaaatttataaaagatggataatcctgggattaaataaaatagtaagaatgtatagccctaccagcattctggacataagacaaggaccaaaagaaccctttagagactatgtag

SEQ ID NO: 44 - DNA, 50 bp, Artificial Sequence; identical to SEQ ID NO: 23
  HIV Capture Probe

SEQ ID NO: 45 - DNA, 50 bp, Artificial Sequence
  HIV CAPTURE PROBE
ccacctatcccagtaggagaaatctataaaagatggataatcctgggatt

SEQ ID NO: 46 - DNA, 258 bp, Artificial Sequence
  HIV PCR product
tgaagcttgctcggctcttagagttttatagaaccggtctacatagtctctaaagggttcttttggtccttgtcttatgtccagaatgctggtagggctatacattcttactattttatttaatcccaggattatccatcttttataaatttctcctactgggataggtggattatttgtcatccatcctatttgttcctgaagggtactagtagttcctgctatgtcacttccccttggttctctcatctggcctgg

SEQ ID NO: 47 - protein, 13 aa, Artificial Sequence
  ALFA-tag
SRLEEELRRRLTE

SEQ ID NO: 48 - protein, 15 aa, Artificial Sequence
  AviTag
GLNDIFEAQKIEWHE

SEQ ID NO: 49 - protein, 4 aa, Artificial Sequence
  C-Tag
EPEA

SEQ ID NO: 50 - protein, 26 aa, Artificial Sequence
  Calmodulin-Tag
KRRWKKNFIAVSAANRFKKISSSGAL

SEQ ID NO: 51 - protein, 6 aa, Artificial Sequence
  Polyglutamate tag
EEEEEE

SEQ ID NO: 52 - protein, 13 aa, Artificial Sequence
  E-Tag
GAPVPYPDPLEPR

SEQ ID NO: 53 - protein, 8 aa, Artificial Sequence
  FLAG-Tag
DYKDDDDK

SEQ ID NO: 54 - protein, 9 aa, Artificial Sequence
  HA-Tag
YPYDVPDYA

SEQ ID NO: 55 - protein, 10 aa, Artificial Sequence
  Myc-Tag
EQKLISEEDL

SEQ ID NO: 56 - protein, 18 aa, Artificial Sequence
  NETtag
TKENPRSNQEESYDDNES

SEQ ID NO: 57 - protein, 9 aa, Artificial Sequence
  Rho1D4-Tag
TETSQVAPA

SEQ ID NO: 58 - protein, 15 aa, Artificial Sequence
  S-Tag
KETAAAKFERQHMDS

SEQ ID NO: 59 - protein, 38 aa, Artificial Sequence
  SBP-Tag
MDEKTTGWRGGHVVEGLAGELEQLRARLEHHPQGQREP

SEQ ID NO: 60 - protein, 13 aa, Artificial Sequence
  Softag 1
SLAELLNAGLGGS

SEQ ID NO: 61 - protein, 8 aa, Artificial Sequence
  Softag 3
TQDPSRVG

SEQ ID NO: 62 - protein, 12 aa, Artificial Sequence
  Spot-Tag
PDRVRAVSHWSS

SEQ ID NO: 63 - protein, 8 aa, Artificial Sequence
  Strep-Tag
WSHPQFEK

SEQ ID NO: 64 - protein, 8 aa, Artificial Sequence
  Strep-Tag
WSHPQFEK

SEQ ID NO: 65 - protein, 11 aa, Artificial Sequence
  T7-Tag
MASMTGGQQMG

SEQ ID NO: 66 - protein, 6 aa, Artificial Sequence
  TC-Tag
CCPGCC

SEQ ID NO: 67 - protein, 10 aa, Artificial Sequence
  Ty1 Tag
EVHTNQDPLD

SEQ ID NO: 68 - protein, 14 aa, Artificial Sequence
  V5 Tag
GKPIPNPLLGLDST

SEQ ID NO: 69 - protein, 11 aa, Artificial Sequence
  VSV-Tag
YTDIEMNRLGK

SEQ ID NO: 70 - protein, 8 aa, Artificial Sequence
  Xpress Tag
DLYDDDDK

SEQ ID NO: 71 - protein, 16 aa, Artificial Sequence
  Isopeptag
TDKDMTITFTNKKDAE

SEQ ID NO: 72 - protein, 13 aa, Artificial Sequence
  SpyTag
AHIVMVDAYKPTK

SEQ ID NO: 73 - protein, 12 aa, Artificial Sequence
  SnoopTag
KLGDIEFIKVNK

SEQ ID NO: 74 - protein, 23 aa, Artificial Sequence
  DogTag
DIPATYEFTDGKHYITNEPIPPK

SEQ ID NO: 75 - protein, 13 aa, Artificial Sequence
  SdyTag
DPIVMIDNDKPIT